(19) United States
(12) Patent Application Publication
(10) Pub. No.: US 2026/0232601 A1
(43) Pub. Date: Aug. 13, 2026

(54) COMPOSITIONS AND METHODS FOR DELIVERING CARGO TO CELLS

(71) Applicant: The University of North Carolina at Chapel Hill, Chapel Hill, NC (US)

(72) Inventors: Owen Fenton, JR., Chapel Hill, NC (US); Yutian Ma, Cambridge, MA (US); Palas Tiwade, New Brunswick, NJ (US)

(21) Appl. No.: 19/474,851

(22) PCT Filed: Apr. 12, 2024
(86) PCT No.: PCT/US2024/024260

§ 371 Date: Oct. 13, 2025

Related U.S. Application Data
(60) Provisional application No. 63/496,140, filed on Apr. 14, 2023.

(51) Int. Cl. A61K 9/51; A61K 31/7105; C08G 73/02

(52) U.S. Cl. CPC A61K 9/5146; A61K 31/7105; C08G 73/0206

(57) ABSTRACT

Disclosed herein are functionalized polyethylenimine compounds for use as nanoparticles. Methods of making the compounds and nanoparticles comprising a cargo and methods of delivering a nanoparticle comprising a cargo are provided. The nanoparticles of the present invention can be utilized in methods of delivering cargo in a tissue specific manner, providing low toxicity and efficient release of cargo in the cell.

### CROSS-REFERENCE TO RELATED APPLICATIONS

This application claims the benefit of U.S. Provisional Application Ser. No. 63/496,140, filed Apr. 14, 2023, the entire contents of which are incorporated by reference herein.

### FIELD OF INVENTION

The invention relates to functionalized polyethylenimine compounds, nanoparticles comprising the functionalized polyethylenimine compounds and a cargo molecule, and methods of making them. The invention further relates to methods of using the novel nanoparticles to deliver cargo in a tissue-specific manner.

### BACKGROUND

Nucleic acid sequences can be rapidly engineered to silence, augment, or replace defective genes in the body. Accordingly, the usage of nucleic acids as drugs has emerged as an attractive strategy to prevent and treat disease. However, nucleic acids, such as RNA, are not effective as a therapeutic when delivered alone.

Messenger RNA (mRNA) delivery platforms often facilitate protein expression in the liver following intravenous injection and have been optimized for use in normally oxygenated cells. However, there is a growing need for mRNA therapy in diseases affecting non-liver organs, such as the lungs. Additionally, many diseases are characterized by hypoxia, a state of abnormally low oxygenation in cells and tissues that can reduce the efficacy of mRNA therapies by upwards of 80%.

Novel synthetic materials that can target nucleic acids and other cargos to a variety of cells and specific tissue types would find broad therapeutic application in the field. Additionally, developing platform technologies that can deliver mRNA to the lungs whose delivery properties can be readily tuned to restore lost efficacy in hypoxic cells could inform the development of next-generation therapies for diseases.

### INVENTION SUMMARY

The present invention is based, in part, on the development of functionalized polyethyleneimine (PEI) compounds useful as nanoparticles for the delivery of cargo. In one embodiment, PEI compounds are provided comprising a plurality of amine groups, wherein at least one amine group comprises a nitrogen atom directly bonded to a functional group according to Formula I:

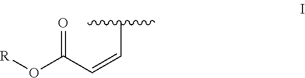

wherein R is C₆ to C₁₈ alkanyl, alkenyl or alkynyl.

Compounds disclosed herein can be formed by reacting a PEI with an alkynoate according to Formula II:

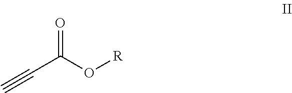

to thereby form a functionalized PEI compound, wherein R is C₆ to C₁₈ alkanyl, alkenyl or alkynyl, e.g., a C₆, C₇, C₈, C₉, C₁₀, C₁₁, C₁₂, C₁₃, C₁₄, C₁₅, C₁₆, C₁₇, or C₁₈ is alkanyl, alkenyl or alkynyl, or any range therein. In some embodiments, R is a C₆ to C₁₃ alkanyl, alkenyl or alkynyl.

The polyethyleneimines may be branched or unbranched. In an aspect, the PEI is branched with a number average molecular weight (Mn) of about 400 to about 750,000 g/mol. In one aspect, the PEI is branched with a Mn of about 600 g/mol. In one aspect, the PEI is linear with a Mn of about 400 to about 30,000 g/mol.

The alkynoate reacted with the PEI can be provided at a ratio of about 1 to about 20 molar equivalents to the PEI or any range therein, e.g., about at 2.5, 5, 7.5 or 10 molar equivalents to the PEI.

Nanoparticles comprising the functionalized polyethylenimine compounds disclosed herein and a cargo are also provided. In an aspect, the nanoparticle is not a lipid nanoparticle. In an aspect, the nanoparticle further comprises a polyethylene glycol (PEG).

The nanoparticle can comprise a cargo that is a biologically active agent, imaging agent, or therapeutic agent. In an aspect, the cargo is a nucleic acid, a protein, a complex of a nucleic acid and a protein, a carbohydrate, a lipid, or a small molecule. In one aspect, the cargo nucleic acid is an RNA, for example, an mRNA, antisense oligonucleotide, or siRNA. In one embodiment, the cargo may be a gene modulating agent. The gene modulating agent may be a gene editing system comprising a CRISPR-Cas system, a zinc finger nuclease, a TALEN, or a meganuclease. The nanoparticle can further comprise an excipient, for example a nucleoside triphosphate (NTP) (e.g., adenosine triphosphate (ATP)), or a derivative, metabolite, analog, or precursor thereof.

Methods of delivering a cargo to a target cell are provided, comprising contacting the target cell with a nanoparticle described herein. The contacting the target cell may occur in vivo, ex vivo, or in vitro. In an aspect, the target cell is a lung cell, a spleen cell, a liver cell, a cardiac cell, a kidney cell, or a reproductive organ cell.

Methods of delivering a cargo in a tissue specific manner are provided, comprising contacting the tissue with the nanoparticle described herein. In one aspect, the tissue is lung, spleen, liver heart, kidney, or reproductive organs.

Methods of making the modified PEI compound are provided. In one embodiment, the method comprises adding a solution of the alkynoate in a solvent to the PEI to make a mixture, stirring the mixture at room temperature thereby forming the compound in the solvent, and evaporating the solvent, thereby producing the compound. In one aspect, the solvent is dichloromethane. The steps can be performed without a catalyst or initiator. In one aspect, the methods provide a yield that is about 95%, 96%, 97%, 98%, 99% or more.

Methods of making a nanoparticle are provided. Methods of making a nanoparticle can comprise adding a cargo to a modified PEI compound of the invention, wherein the nanoparticle self-assembles via electrostatic interactions between the cargo and the compound. The method can further comprise the step of adding PEG. The cargo may be a biologically active agent, imaging agent, or therapeutic agent. The cargo may be a nucleic acid, a protein, a complex of a nucleic acid and a protein, a carbohydrate, a lipid, or a small molecule. The cargo nucleic acid can be an RNA, for example an mRNA, antisense oligonucleotide, or siRNA. The cargo can be a gene modulating agent. The gene modulating agent can be a gene editing system comprising a CRISPR-Cas system, a zinc finger nuclease, a TALEN, or a meganuclease.

These and other aspects of the invention are set forth in more detail in the description of the invention below.

### BRIEF DESCRIPTION OF THE DRAWINGS

FIG. 1. Imaging showing luciferase mRNA injected at 0.5 mg/kg in mouse shows no expression of mRNA in mouse organs.

FIG. 2. Schematic of approach to formulate customizable nanoparticles (NP) utilizing synthetic materials for RNA delivery.

FIG. 3. Synthesis scheme for compound library of PEI functionalized with C₆-C₁₈ alkynoates.

FIGS. 4A-4D. Reaction Scheme, Mass Spectrometry Data and ¹H NMR of functionalized PEI compound with (A) 2.5 equivalents C₆ alkynoate, (B) 5 equivalents C₆ alkynoate, (C) 7.5 equivalents C₆ alkynoate, and (D) 10 equivalents C₆ alkynoate.

FIGS. 5A-5D. Reaction Scheme, Mass Spectrometry Data and ¹H NMR of functionalized PEI compound with (A) 2.5 equivalents C₇ alkynoate, (B) 5 equivalents C₇ alkynoate, (C) 7.5 equivalents C₇ alkynoate, and (D) 10 equivalents C₇ alkynoate.

FIGS. 6A-6D. Reaction Scheme, Mass Spectrometry Data and ¹H NMR of functionalized PEI compound with (A) 2.5 equivalents C₈ alkynoate, (B) 5 equivalents C₈ alkynoate, (C) 7.5 equivalents C₈ alkynoate, and (D) 10 equivalents C₈ alkynoate.

FIGS. 7A-7D. Reaction Scheme, Mass Spectrometry Data and ¹H NMR of functionalized PEI compound with (A) 2.5 equivalents C₉ alkynoate, (B) 5 equivalents C₉ alkynoate, (C) 7.5 equivalents C₉ alkynoate, and (D) 10 equivalents C₉ alkynoate.

FIGS. 8A-8D. Reaction Scheme, Mass Spectrometry Data and ¹H NMR of functionalized PEI compound with (A) 2.5 equivalents C₁₀ alkynoate, (B) 5 equivalents C₁₀ alkynoate, (C) 7.5 equivalents C₁₀ alkynoate, and (D) 10 equivalents C₁₀ alkynoate.

FIGS. 9A-9D. Reaction Scheme, Mass Spectrometry Data and ¹H NMR of functionalized PEI compound with (A) 2.5 equivalents C₁₁ alkynoate, (B) 5 equivalents C₁₁ alkynoate, (C) 7.5 equivalents C₁₁ alkynoate, and (D) 10 equivalents C₁₁ alkynoate.

FIGS. 10A-10D. Reaction Scheme, Mass Spectrometry Data and ¹H NMR of functionalized PEI compound with (A) 2.5 equivalents C₁₂ alkynoate, (B) 5 equivalents C₁₂ alkynoate, (C) 7.5 equivalents C₁₂ alkynoate, and (D) 10 equivalents C₁₂ alkynoate.

FIGS. 11A-11D. Reaction Scheme, Mass Spectrometry Data and ¹H NMR of functionalized PEI compound with (A) 2.5 equivalents C₁₃ alkynoate, (B) 5 equivalents C₁₃ alkynoate, (C) 7.5 equivalents C₁₃ alkynoate, and (D) 10 equivalents C₁₃ alkynoate.

FIGS. 12A-12D. Dynamic Light Scattering (DLS) particle characterization data of particles loaded with mRNA run in triplicate for (A) 2.5 equivalents alkynoate, (B) 5 equivalents alkynoate, (C) 7.5 equivalents alkynoate, and (D) 10 equivalents alkynoate.

FIGS. 13A-13C. Collective transfection data for library of functionalized PEI particles using C₆-C₁₃ alkynoates with variable tail to PEI ratio as detailed in FIGS. 4A-11D, and 50 ng mRNA per well in U251 cells, charts translation (upper panel) and cell viability (lower panel) for (A) Trial 1, (B) Trial 2, and (C) Trial 3.

FIGS. 14A-14B. Example particles comprising 5C₆, 5C₇, and 10C₈ loaded with 50 ng mRNA, performance compared to Moderna formulation in (A) translation and (B) U251 cell viability; positive control: Lipofectamine and PBS.

FIGS. 15A-15B. Collective transfection data for library of functionalized PEI particles using C₆-C₁₃ alkynoates as detailed in FIGS. 4A-11D with variable tail to PEI ratio and 50 ng mRNA per well in Madin-Darby canine kidney (MDCK) cells, charts translation (upper panel) and cell viability (lower panel).

FIGS. 16A-16D. DLS particle characterization data of particles loaded with mRNA, ethanol into aqueous, in MDCK cell line for (A) 2.5 equivalents of C₆-C₁₃ alkynoate modified PEI, (B) 5 equivalents C₆-C₁₃ alkynoate modified PEI, (C) 7.5 equivalents a C₆-C₁₃ alkynoate modified PEI, and (D) 10 equivalents C₆-C₁₃ alkynoate modified PEI.

FIGS. 17A-17D. In vivo intravenous screening of example particles of C₆ alkynoate modified PEI particles formulated with 0.25% PEG and luciferase mRNA and injected into mice at 0.5 mg/kg. (A) 5 equivalents of C₆ alkynoate to PEI shows activity in lung and spleen tissues of mice; (B) 7.5 equivalents of C₆ alkynoate to PEI shows activity in lung and spleen tissues of mice; (C) Change in mouse weight before and 24 hours post-injection of particles, with comparison of Moderna particles; (D) Measured luminescence for mouse organs shows liver expression for Moderna injection, lung and spleen for example formulations of present invention, and no tissue expression for mRNA delivered alone.

FIGS. 18A-18D. DLS particle characterization data of particles with PEG loaded with mRNA, ethanol into aqueous, in U251 cell line for (A) 2.5 equivalents of C₆-C₁₃ alkynoate modified PEI, (B) 5 equivalents C₆-C₁₃ alkynoate modified PEI, (C) 7.5 equivalents a C₆-C₁₃ alkynoate modified PEI, and (D) 10 equivalents C₆-C₁₃ alkynoate modified PEI.

FIG. 19. Collective transfection data for library of C₆-C₁₃ alkynoate modified PEI particles formulated with 0.25% PEG and luciferase mRNA with 0.25% PEG with variable tail to PEI ratio as detailed in FIGS. 4A-12D, and 50 ng mRNA per well in U251 cells; translation measured.

FIGS. 20A-20D. In vivo intravenous screening trial 2 of example particles of alkynoate modified PEI particles formulated with 0.25% PEG and luciferase mRNA and injected into mice. (A) DLS data for example particles loaded with mRNA; after dialysis; (B) 5 equivalents of C₆ alkynoate to PEI modified particles and shows activity in lung and spleen tissues of mice, Moderna particles included for comparison; (C) 7.5 equivalents and 10 equivalents of alkynoate to PEI modified particles; (D) change in mouse weight before injection and 24 hours post-injection of particles, with comparison of Moderna particles.

FIG. 21. Schematic illustration of the goal of Experiment 4.

FIGS. 22A-22O. Formulation development, characterization, and optimization of 5C6 Polymeric nanoparticles (PNPs) in A549 cells. (A) Structure of novel 5C6 polymer (B) Schematic illustration of 5C6 PNPs formulation with TEM image of the 5C6 PNPs. (C) Schematic illustration used for optimizing 5C6 PNPs (D) Size, (E) Zeta potential, (F) mRNA encapsulation efficiency, and (G) in vitro luciferase expression in A549 cells of 5C6 PNPs obtained through PEG percentage change. (H) Size, (I) Zeta potential, (J) mRNA encapsulation efficiency, and (K) in vitro luciferase expression in A549 cells of 5C6 PNPs obtained through variation in PEG head group. (L) Size, (M) Zeta potential, (N) mRNA encapsulation efficiency, and (O) in vitro luciferase expression in A549 cells of 5C6 PNPs obtained through variation in PEG molecular weight. In in vitro studies, cells were dosed at 50 ng of mRNA per 10,000 cells and evaluated for luciferase expression after 24 hours. Cells treated with Moderna formulation, naked mRNA, and PBS were used as benchmarks and controls. (Data are shown as the mean±SD, n=3, ****p<0.0001, ***p<0.001, **p<0.01, *p<0.05 and ns p>0.05 difference from Moderna with 95% of confidence interval from unpaired t-test).

FIGS. 23A-23G. In vitro mechanism investigation of 5C6 PNPs in A549 cells. (A) Schematic representation of the mechanisms studied. (B) Cellular association quantification of A549 cells treated with 5C6 PNPs entrapping Cy5 labeled mRNA at 2 h and 24 h using flow cytometry. (Data shown as the mean±SD, n=3, ****p<0.0001 difference from Moderna with 95% confidence interval from unpaired t-test) (C) Schematic representation of endocytosis mechanisms studied (phagocytosis and macropinocytosis are inhibited by cytochalasin D, macropinocytosis is inhibited by EIPA, caveolae-mediated endocytosis is inhibited by pitstop 2, and clathrin-mediated endocytosis is inhibited by filipin). (D) Percent reduction in the uptake of 5C6 PNPs entrapping Cy5 labeled mRNA in A549 cells inhibited with cytochalasin D, EIPA, pitstop 2, and filipin as measured by flow cytometry. Quantification of the endosomal escape properties of 5C6 PNPs. (E) Schematic representation of the endosomal escape assay. (F) Representative confocal images of A549 cells treated with 5C6 PNPs entrapping Cy5 labeled mRNA (dark gray) or Moderna LNPs entrapping Cy5 labeled mRNA (dark gray) and stained for endo/lysosomes (light gray) and nucleus (gray). Scale bars represent 10 μm. (G) Pearson Correlation Coefficient (PCC) analysis of A549 cells treated with 5C6 PNPs corresponding with FIG. 23F. (Data presented as the mean±SD, N>20, *p<0.05 and ns p>0.05 difference from Moderna with 95% confidence interval from unpaired t-test).

FIGS. 24A-24D. (A) Reduction in the percentage of GFP-positive A549 cells following treatment with inhibitor followed by 5C6 PNPs entrapping GFP mRNA (inhibitor=bafilomycin A₁, a V-type ATPase inhibitor of proton sponge effect; percent reduction is relative to A549 cells treated only with 5C6 PNPs entrapping GFP mRNA). (B) Reduction in percentage of GFP-positive A549 cells following treatment with bafilomycin A₁ (to inhibit proton sponge effect) and respective inhibitors followed by 5C6 PNPs entrapping GFP mRNA (inhibitors=cytochalasin D−an inhibitor of phagocytosis and macropinocytosis; EIPA−an inhibitor of macropinocytosis; pitstop 2−an inhibitor of caveolae-mediated endocytosis; or filipin an inhibitor of clathrin-mediated endocytosis; percent reduction is relative to A549 cells treated only with 5C6 PNPs entrapping GFP mRNA). (C) Schematic representation of proton sponge inhibition assay using bafilomycin A₁ and diffusion of calcein fluorescent dye. (D) Representative confocal laser microscopy images of A549 cells treated with 1000 ng mL⁻¹ FLuc mRNA in 5C6 PNPs or Moderna LNPs and calcein (left), calcein with bafilomycin A₁ (middle), or calcein with bafilomycin A₁ and cytochalasin D (right), Scale bars are 50 μm. (Data shown as the mean SD, n=3, ****p<0.0001 difference from Moderna with 95% confidence interval from unpaired t-test)

FIGS. 25A-25F. Effect of hypoxia on the expression of mRNA encoded FLuc delivered by 5C6 PNPs. (A) Schematic representation of the effect of hypoxia on protein expression. (B) Experimental conditions of hypoxia experiments. Normoxia=FLuc expression quantified in A549 cells that have been incubated under normoxia for 24 h, then treated with each respective 5C6 PNP, then incubated under normoxia for an additional 24 h; Hypoxia=FLuc expression quantified in A549 cells that have been incubated under hypoxia for 24 h, then treated with each respective 5C6 PNP, then incubated under hypoxia for an additional 24 h. (C-E) FLuc mRNA expression of A549 cells in normoxia (21% oxygen) and hypoxia (1% oxygen) at (C) 50 ng, (D) 100 ng, and (E) 200 ng mRNA doses. (F) Heat map of reduction in protein expression in hypoxic conditions relative to normoxic conditions across different 5C6 PNP formulations and FLuc mRNA doses. (Data are shown as the mean±SD, n=3, ****p<0.0001, ***p<0.001, **p<0.01, *p<0.05 and ns p>0.05 with 95% of confidence interval from paired t-test).

FIGS. 26A-26H. Improving 5C6 PNP efficacy using an ATP co-delivery strategy. (A) Schematic representation of formulation of 5C6 ATP (+) PNPs. (B) mRNA and ATP encapsulation efficiencies of ATP (+) formulations. (C) Size, (D) charge, and (E) PDI of the 5C6 ATP (−) vs 5C6 ATP (+) formulations. (F) Experimental conditions of normoxia vs hypoxia experimental design conditions. Normoxia=FLuc expression quantified in A549 cells that have been incubated under normoxia for 24 h, then treated with each respective 5C6 ATP (−) or 5C6 ATP (+) PNPs, then incubated under normoxia for an additional 24 h; Hypoxia=FLuc expression quantified in A549 cells that have been incubated under hypoxia for 24 h, then treated with each respective 5C6 ATP (−) or 5C6 ATP (+) PNPs, then incubated under hypoxia for an additional 24 h. (G-H) FLuc mRNA expression of A549 cells treated with 5C6 ATP (−) or 5C6 ATP (+) PNPs under (G) normoxia and (H) hypoxia. (Data are shown as the mean±SD, n=3, ****p<0.0001, ***p<0.001, **p<0.01, *p<0.05 and ns p>0.05 with 95% of confidence interval from paired t-test).

FIGS. 27A-27D. In vivo evaluation of 5C6 PNPs. (A) Representative luminescence biodistribution of 5C6 PNPs with FLuc mRNA following intravenous (IV) injection, Moderna LNPs (positive control), naked FLuc mRNA or PBS (negative control) via intravenous (IV) injection (n=3) at 0.25 mg/kg mRNA. (B) Quantification of FLuc signal in the lungs of mice treated with 5C6 PNPs. Data presented as mean±standard deviation (n=3), ****p<0.0001, **p<0.01, *p<0.05, and ns p>0.05 with a 95% confidence interval from unpaired t-test. (C) The associated percentage of total FLuc signal in the pancreas, spleen, liver, kidneys, uterus/ovaries, lung, and heart of mice treated with IV doses of 5C6 PNPs. (Data are shown as the mean±SD, n=3, ****p<0.0001, as compared with the Moderna formulation with a two-way ANOVA). (D) Representative histology images of the liver, spleen, and lung of mice after treatment with 5C6 PNP via IV injection (n=3). Scale bars are 50 μm.

FIGS. 28A-28B. (A) Reaction of scheme C6 alkynoate tail synthesis via Fisher esterification. (B) 1H NMR of C6 alkynoate tail.

FIG. 29. Reaction scheme, ¹H NMR and Mass Spectrum of 5C6.

FIG. 30. DLS of 5C6 particles without PEG pre-dialysis and post dialysis against PBS. Post-dialysis particle size measurements in microns indicate instability of particles.

FIGS. 31A-31B. (A) mRNA expression by 5C6 formulation (B) cell viability evaluated at different N/P ratios. Data are shown as the mean±SD, n=3, *p<0.05 and ns p>0.05 with 95% of confidence level from unpaired t-test).

FIGS. 32A-32C. Cell viability of (A) 5C6 nanoparticles formulated at different N/P ratio (B) Cell viability of 5C6 nanoparticles formulated with different PEG head groups (C) Cell viability of 5C6 nanoparticles formulated with different PEG molecular weight at 24 h. (Data corresponds to FIGS. 22G, 22K, 22O; Data are shown as the mean SD, n=3).

FIGS. 33A-33E. TNS curves of (A) Moderna (B) 5C6 C14 1K (C) 5C6 C14 2K (D) 5C6 C14 3K and (E) 5C6 C14 5K formulations.

FIG. 34. Stability of 5C6 nanoparticles formulated with different PEG molecular weight (1 k, 2 k, 3 k and 5 k) and Moderna lipid nanoparticles after the incubation of in pH 7.4, pH 5, DMEM+10% FBS solution in either 4° C. or 37° C. for 72 h.

FIG. 35. Flow cytograms showing the gating of control (unstained cells) for cell association and endocytosis mechanism in A549 cells.

FIG. 36. Flow cytograms showing the gating of control (unstained cells), Moderna and 5C6 formulations for cell inhibitor studies in A549 cells.

FIGS. 37A-37C. Cell viability of 5C6 nanoparticles formulated with different PEG molecular weight (1K, 2K, 3K and 5K) and Moderna lipid nanoparticles at (A) 50 ng, (B) 100 ng and (C) 200 ng dose in normoxia and hypoxia for 24 h. (Data corresponds to FIGS. 25C, 25D, 25E; Data are shown as the mean±SD, n=3).

FIGS. 38A-38B. Cell viability of 5C6 nanoparticles formulated with different PEG molecular weight (1 k, 2 k, 3 k and 5 k) and Moderna lipid nanoparticles with or without ATP under (A) normoxia and (B) hypoxia for 24 h. (Data corresponds to FIG. 6 g and h; Data are shown as the mean±SD, n=3).

FIG. 39. Percentage weight change of mice 24 hours post treatment.

FIG. 40. Blood panel results of liver and kidney function tests from 0.5 mg/kg dose of 5C6 injected intravenously demonstrate LNPs are well-tolerated.

FIG. 41. Representative images of histology from representative sectioning shows tolerability in lung, liver and spleen tissues with PBS and 5C6 formulation.

FIG. 42. Exemplary cryo-electron microscopy image of 5C6 indicating multilamellar structure of the particles with spherical morphology.

FIG. 43. Results of stability studies for 5C6 (left) and 7.5C6 (right) complexed with mRNA with and without fetal bovine serum. 3 μL of sample was tested every 24 hours until 48 hours, then taken every 48 hours for size measurement on DLS.

FIG. 44. Imaging of concentration in mice organs resulting from Fluc mRNA intratracheally administered as naked mRNA, in Moderna LNP formulation, and 5C6 formulation taken 24 hours post dosing.

FIG. 45. Measurement of effect of hypoxia in A549 cell line with varying reaction equivalents of R tail length of C6-C13 in FLuc mRNA LNPs; Normoxia 21% O₂, Hypoxia 1% O₂, RNA dose 0.5 μg/mL.

FIG. 46. Tunability of protein expression with the addition of ATP to mRNA encapsulated LNP, resulting in approximately 2-fold increase; Normoxia 21% O₂, Hypoxia 1% O₂, RNA dose 0.5 μg/mL.

FIG. 47. Imaging showing 5C6 LNPs deliver multiple types of mRNA irrespective of coding sequence; A549 cell line, mRNA dose 0.5 μg/mL, 24 hours.

FIG. 48. Results of PTEN mRNA dose response showing anti-cancer effect of PTEN mRNA delivery. 40,000 A549 (NSCLC) cells; 5C6 C14 2K particles evaluated via flow cytometry; ELISA—stats against media.

FIGS. 49A-49C. Images of lung cancer model, upper row showing tumor location in mice, lower row showing particle location. Images taken after second dose after administration of (A) PBS; (B) 0.2 mol % DiD; (C) 1 mol % DiD.

FIG. 50. Measurement of accumulation of 5C6 LNPs signal in lung tumor in orthotopic mouse model with 0.25 mg/kg Cy5-GFP mRNA, DiD.

### DETAILED DESCRIPTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. In addition, any references cited herein are incorporated by reference in their entireties.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Amino acids are represented herein in the manner recommended by the IUPAC-IUB Biochemical Nomenclature Commission, or (for amino acids) by either the one-letter code, or the three-letter code, both in accordance with 37 C.F.R. § 1.822 and established usage.

Except as otherwise indicated, standard methods known to those skilled in the art may be used for cloning genes, amplifying and detecting nucleic acids, and the like. Such techniques are known to those skilled in the art. See, e.g., Sambrook et al., Molecular Cloning: A Laboratory Manual 4th Ed. (Cold Spring Harbor, NY, 2012); Ausubel et a. Current Protocols in Molecular Biology (Green Publishing Associates, Inc. and John Wiley & Sons, Inc., New York).

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, ±5%, ±1%, ±0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “consists essentially of” (and grammatical variants), as applied to a polypeptide or polynucleotide sequence of this invention, means a polypeptide or polynucleotide that consists of both the recited sequence (e.g., SEQ ID NO) and a total of ten or less (e.g., 1, 2, 3, 4, 5, 6, 7, 8, 9, or 10) additional amino acids on the N-terminal and/or C-terminal ends of the recited sequence or additional nucleotides on the 5′ and/or 3′ ends of the recited sequence such that the function of the polypeptide or polynucleotide is not materially altered. The total of ten or less additional amino acids or nucleotides includes the total number of additional amino acids or nucleotides on both ends added together. The term “materially altered,” as applied to polypeptides of the invention, refers to an increase or decrease in biological activities/properties (e.g., remodeling activity) of at least about 50% or more as compared to the activity of a polypeptide consisting of the recited sequence.

As used herein, the term “polypeptide” encompasses both peptides and proteins, unless indicated otherwise.

The terms “polynucleotide,” “nucleic acid,” “nucleic acid molecule,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, genomic DNA, chimeras of RNA and DNA, isolated DNA of any sequence, isolated RNA of any sequence, synthetic DNA of any sequence (e.g., chemically synthesized), synthetic RNA of any sequence (e.g., chemically synthesized), nucleic acid probes and primers. A polynucleotide can comprise modified nucleotides, such as methylated nucleotides and nucleotide analogs or derivatives (e.g., inosine or phosphorothioate nucleotides). Such nucleotides can be used, for example, to prepare nucleic acid molecules that have altered base-pairing abilities or increased resistance to nucleases.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold and/or can be expressed in the enhancement and/or increase of a specified level and/or activity of at least about 1%, 5%, 10%, 15%, 25%, 35%, 40%, 50%, 60%, 75%, 80%, 90%, 95% or more.

The term “inhibit” or “reduce” or grammatical variations thereof as used herein refers to a decrease or diminishment in the specified level or activity of at least about 1, 5, 10, 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

The term “contact” or grammatical variations thereof refers to bringing two or more substances in sufficiently close proximity to each other for one to exert a biological effect on the other.

It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. In a broad aspect, the permissible substituents include acyclic and cyclic, branched and unbranched, carbocyclic and heterocyclic, aromatic and nonaromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Non-limiting examples of optional substituents as referred to herein include halogen, alkyl, aralkyl, heteroalkyl, alkenyl, alkynyl, cycloalkyl, hydroxyl, alkoxy, amino, amido, nitro, cyano, amido, carbonyl, carboxyl, silyl, ether, alkylthio, sulfonyl, sulfonamido, ketone, aldehyde, ester, heterocyclyl, aryl, and heteroaryl.

As used herein, the term “alkyl”, used either alone or in compound words such as “haloalkyl” includes straight-chain or branched C₁-C₂₀ alkyl, such as methyl, ethyl, n-propyl, i-propyl, or the different butyl, pentyl or hexyl isomers, etc.

As used herein, the term “alkenyl”, used either alone or in compound words such as “haloalkenyl” includes straight-chain or branched C₁-C₂₀ alkyl containing at least one double bond.

As used herein, the term “alkynyl”, used either alone or in compound words such as “haloalkynyl” includes straight-chain or branched C₁-C₂₀ alkyl containing at least one triple bond.

As used herein, “unsaturated” refers to compounds or structures having at least one degree of unsaturation (e.g., at least one double or triple bond).

Substituents around a carbon-carbon double bond alternatively can be referred to as “cis” or “trans,” where “cis” represents substituents on the same side of the double bond and “trans” represents substituents on opposite sides of the double bond. The arrangement of substituents around a carbocyclic ring can also be designated as “cis” or “trans.” The term “cis” represents substituents on the same side of the plane of the ring, and the term “trans” represents substituents on opposite sides of the plane of the ring. Mixtures of compounds wherein the substituents are disposed on both the same and opposite sides of plane of the ring are designated “cis/trans.”

All chiral, diastereomeric, racemic, and geometric isomeric forms of a structure are intended, unless specific stereochemistry or isomeric form is specifically indicated. All processes used to prepare compounds and intermediates made therein are encompassed by the present disclosure. All tautomers of shown or described compounds are also encompassed by the present disclosure.

When any variable (e.g., Ri) occurs more than one time in any constituent or formula for a compound, its definition at each occurrence is independent of its definition at every other occurrence. Thus, for example, if a group is shown to be substituted with one or more Ri moieties, then Ri at each occurrence is selected independently from the Markush group recited for Ri. Also, combinations of substituents and/or variables are permissible, but only if such combinations result in stable compounds within a designated atom's normal valency.

A “subject” may be any vertebrate organism in various embodiments. A subject may be individual to whom an agent is administered, e.g., for experimental, diagnostic, and/or therapeutic purposes or from whom a sample is obtained or on whom a procedure is performed. In some embodiments a subject is a mammal, e.g., a human, non-human primate, lagomorph (e.g., rabbit), or rodent (e.g., mouse, rat). In some embodiments a human subject is a neonate, child, adult, or geriatric subject.

Grammatical variations of “administer,” “administration,” and “administering” to a subject include any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration,” “administration in combination,” “simultaneous administration,” or “administered simultaneously” as used herein, means that the compounds are administered at the same point in time, overlapping in time, or one following the other. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

“Treat,” “treating” and similar terms as used herein in the context of treating a subject refer to providing medical and/or surgical management of a subject. Treatment may include, but is not limited to, administering an agent or composition (e.g., a pharmaceutical composition) to a subject. Treatment is typically undertaken in an effort to alter the course of a disease (which term is used to indicate any disease, disorder, syndrome, or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur, delay the onset of the disease, or to reduce the severity of the disease should it occur. The subject may have been identified as being at risk of developing the disease (e.g., at increased risk relative to the general population or as having a risk factor that increases the likelihood of developing the disease.

The present invention relates to a class of novel synthetic materials that can deliver cargo molecules in vitro and in vivo. The novel synthetic materials comprise functionalized PEI. A majority of the novel compounds outperform the Moderna liponanoparticle (LNP) that is currently used in humans in COVID19 vaccines when administered to cells, as well as compounds that can target different tissue than the Moderna LNP. Methods of making the compounds, nanoparticles comprising cargo molecules, and applications for their use in living systems is also provided.

PEI compounds or polymers, i.e., functionalized PEIs, according to the present invention comprise a plurality of amine groups, wherein at least one amine group comprises a nitrogen atom directly bonded to a functional group according to Formula I:

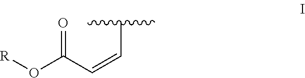

wherein R is C₆ to C₁₈ alkanyl, alkenyl or alkynyl.

The R group of the alkynoate may comprise a saturated or unsaturated C₆ to C₁₈ hydrocarbon or any range therein. Varying degrees of unsaturation can be utilized. In a preferred embodiment, the C₆ to C₁₈ hydrocarbon is a linear alkanyl group. In one embodiment, the R group is a C₆ to C₁₃ alkanyl. In some embodiments, the alkynoate is a C₆ alkynoate, a C₇ alkynoate, a C₈ alkynoate, a C₉ alkynoate, a C₁₀ alkynoate, a C₁₁ alkynoate, a C₁₂ alkynoate, a C₁₃ alkynoate, a C₁₄ alkynoate, a C₁₅ alkynoate, a C₁₆ alkynoate, a C₁₇ alkynoate, or a C₁₈ alkynoate or any range therein.

In an embodiment, the PEI compounds or polymers are prepared by providing the alkynoate at about 1 to about 20 molar equivalents to the PEI. In one aspect, the alkynoate is provided at 2.5, 5, 7.5, or 10 molar equivalents to the PEI or any range therein.

PEI, as used herein, can have a molecular weight in the range between about 400 and 750,000 g/mol. The PEI can be branched or linear. The PEI may be branched with an Mn of about 400 to about 750,000 g/mol. In one embodiment, the PEI is branched with a Mn of about 600 g/mol. In an aspect, the PEI is linear with a Mn of about 400 to about 30,000 g/mol.

Functionalized PEI compounds may comprise 1, 2, 3, 4, 5, 6, 7, 8, 9, 10, 11, 12, 13, 14, 15, 16, 17, 18, 19, 20, 21, 22, 23, 24, 25, 26, 27 or more functional groups on the nitrogen atoms or any range therein.

The compounds of the present invention may be formed by reacting a PEI with an alkynoate according to Formula II.

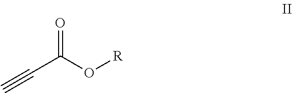

to thereby form a functionalized PEI compound, wherein R is C₆ to C₁₈ alkanyl, alkenyl or alkynyl. In an aspect, one or more functionalized units in the compound are according to

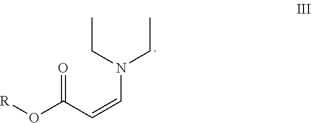

The functionalized PEI compound may comprise 1, 2, 3, 4, 5, 6, 7, 8, 9, 10, 11, 12, 13, 14, 15, 16, 17, 18, 19, 20 or more functionalized units in the compound or any range therein.

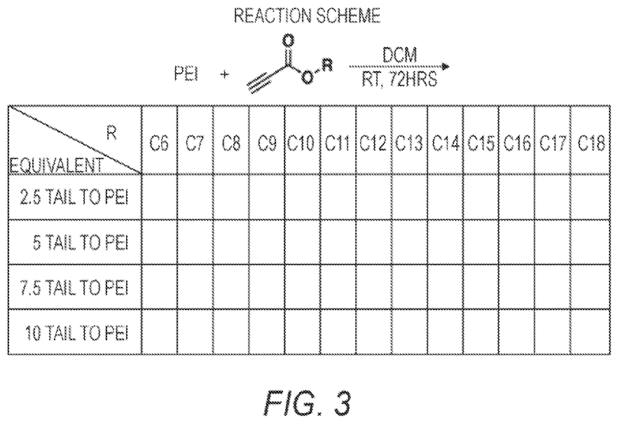

The present invention further comprises compositions, the compositions comprising compounds formed by the reactions described above. In some embodiments, the composition can comprise one or more excipients. In some embodiments, the excipient is a nucleoside trisphosphate (NTP). Exemplary NTP molecules include adenosine triphosphate (ATP), thymidine triphosphate, cytosine triphosphate, guanosine triphosphate and uridine triphosphate. Generally, a nucleoside triphosphate comprises a nitrogenous base (e.g., purine or pyrimidine) a ribose or deoxyribose sugar, and three phosphate groups bound to the sugar molecule. Precursor molecules of NTP include monophosphates such as inosine monophosphate and orotate monophosphate, as well as nucleoside monophosphates and nucleoside diphosphates of the nucleosides. Salts, hydrates, and solvates are encompassed within the NTP molecule, or derivative, metabolite, analog, or precursor thereof. In an embodiment, the NTP molecule, derivative, metabolite, analog, or precursor thereof, utilized in the delivery vectors and methods herein is adenosine 5′-triphosphate (ATP) disodium salt hydrate, adenosine 5′-diphosphate (ADP) disodium salt hydrate, or adenosine 5′-monophosphate (AMP) disodium salt hydrate, or guanosine 5′-triphosphate (GTP) disodium salt hydrate, guanosine 5′-diphosphate (GDP) disodium salt hydrate, or guanosine 5′-monophosphate (GMP) disodium salt hydrate. Additional NTP molecules, derivatives, analogs, and precursors include nucleoside monophosphates, diphosphates, and nucleoside cyclic phosphates. Uracil, cytosine, adenine, guanine and thymine and their derivatives can be utilized in the NTP molecules and its derivatives, analogs, metabolites, and precursors, thus, modified nucleobases, analogs and derivatives are envisioned for use, including halogenated and azotated bases. Additionally, modifications to the ribose sugar are also envisioned for used in the NTP molecule, analog, derivative, metabolite, or precursor thereof, and may include halogenation, methylation, saturation, ring opening, hydroxylation, or dihydroxylation of the sugar. Phosphate groups may comprise protection by polar groups or replacement of a P—O bond by a P—N bond. Accordingly, within the scope of the NTP molecule, derivative, analog, metabolite, or precursor thereof, are molecules with a modified nucleobase, a modified ribose sugar, and/or one, two, three or more phosphate groups that may also be modified. Analogs are known in the art (See Mahmoud et al., Journal of Advanced Pharmacy Research, 1:2, 2, 73-88 (2018), incorporated by reference in its entirety) with additional exemplary structures described in Table 2, Squires, Antiviral Therapy 6 (Suppl. 3) 1-14(2001) and FIG. 3 of Jordheim et al., Nature Review, 12, 447-464(2013), each incorporated herein by reference.

Without being bound by theory, the inclusion of an NTP molecule, derivative, analog, metabolite, or precursor thereof (e.g., ATP or GTP) with mRNA in nanoparticles as described herein can increase expression of protein, for example by 1.5, 1.6, 1., 1.8, 1.9, 2.0 or more times relative to delivery of mRNA without ATP in the nanoparticle.

In some embodiments, the NTP molecule, derivative, analog, metabolite, or precursor thereof can enhance delivery of mRNA from a nanoparticle in hypoxic environments, e.g., less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or at or less than about 1% O₂. In some embodiments, the hypoxic environment is lung tissue or spleen tissue. In some embodiments, the hypoxic environment is tissue with a disease or disorder, acute (e.g., ischemia) or chronic (e.g., chronic kidney disease, cancer). The hypoxia can be generalized or local. Other diseases or disorders that can create a hypoxic environment include, but are not limited to, cardiovascular diseases such as atherosclerosis, pulmonary hypertension, metabolic diseases, asthma, cystic fibrosis, cancer, and kidney diseases.

Methods of making the functionalized PEI compounds can comprise adding a solution of the alkynoate according to formula (I) in a solvent to the chosen PEI to make a mixture, stirring the mixture at room temperature thereby forming the compound in the solvent, and evaporating the solvent, thereby producing the compound. In an aspect, the solvent is dichloromethane. The PEI and the alkynoate can both be provided in dichloromethane. The methods of making the functionalized PEI compounds can preferably be performed without a catalyst or initiator. In an embodiment, the yield from the method is about 95%, 96%, 97%, 98%, 99%, 99.1%, 99.2%, 99.3%, 99.4%, 99.5% or more.

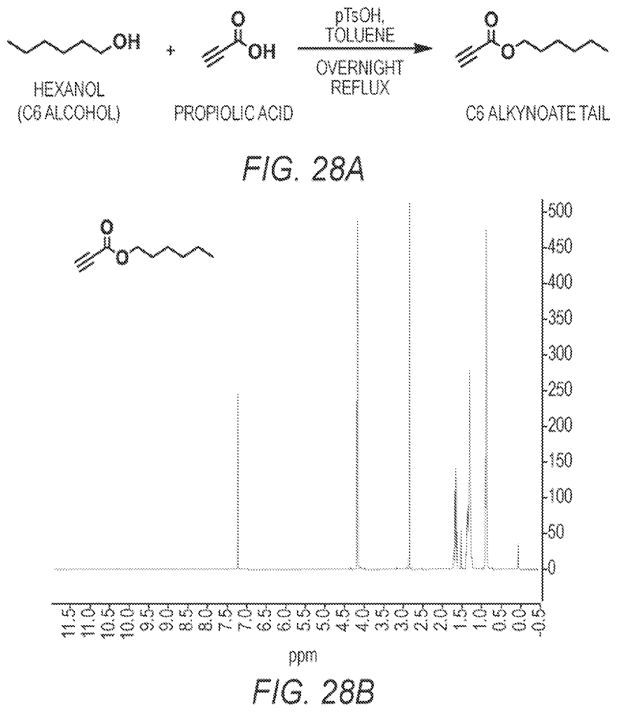

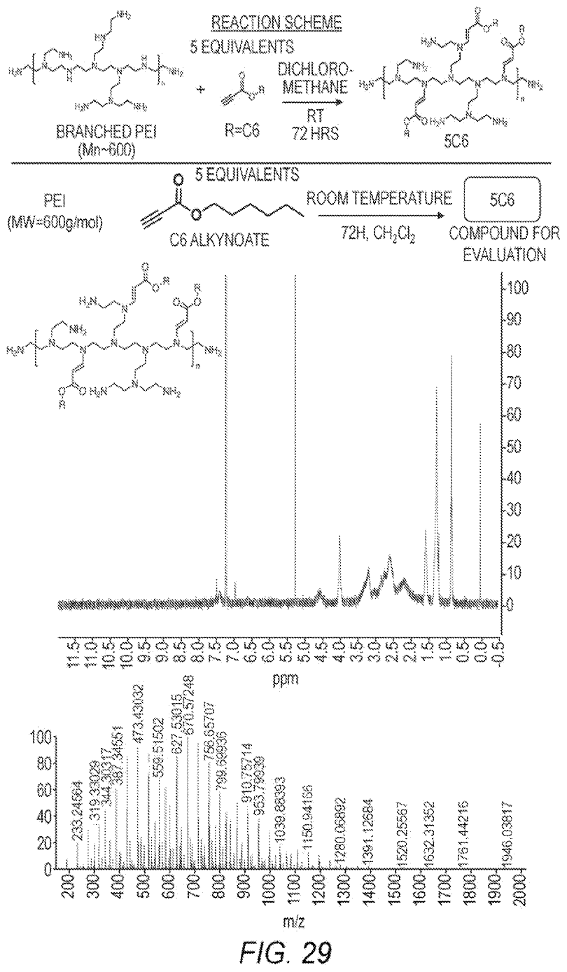

In some embodiments, the alkynoate can be synthesized by reacting a C₆-C₁₈ alcohol with propiolic acid to generate an alkynoate via Fisher esterification. See, e.g., FIG. 28A for example reaction scheme. An exemplary reaction scheme of a solution of the generated alkynoate according to formula (I) in a solvent to a chosen PEI to generate a functionalized PEI compound is depicted in FIG. 29.

Methods of making the functionalized PEI compounds may be performed at temperatures between 20° C. and 30° C., preferably about 23° C. to 27° C. The mixture of the PEI and the alkynoate can be stirred for about 2 hours to about 144 hours, about 10 to about 120 hours, about 24 to about 100 hours, about 48 to about 96 hours, about 60 to 80 hours, or about 72 hours.

The compounds can be utilized to form nanoparticles. In an aspect, the nanoparticle comprises the functionalized PEI compound as described herein, and a cargo.

In some embodiments, the nanoparticle further comprises PEG (e.g., 0.25% PEG) and an mRNA cargo. In some embodiments, the PEG is added to increase stability of the nanoparticle. In some embodiments, the nanoparticles have an average size of about 100-1000 nm, e.g., about 200-950 nm.

In some embodiments, the nanoparticle utilizes a compound formed by the reaction of a branched PEI with a Mₙ of about 600 g/mol with an alkynoate according to Formula II:

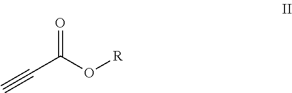

to thereby form functionalized PEI compounds, wherein R is C₆ to C₁₈ alkanyl, alkenyl or alkynyl. In some embodiments, the nanoparticle comprises alkynoate reacted at 2.5, 5, 7.5 or 10 molar equivalents to PEI or any range therein. The nanoparticle can be characterized by its polydispersity index (PDI). In some embodiments, the nanoparticle comprises alkynoate reacted at 2.5 molar equivalents to PEI. In some embodiments, the alkynoate is a C₆ alkynoate; in some embodiments, the nanoparticle has a PDI between about 0.075 and 0.300. In some embodiments, the alkynoate is a C₇ alkynoate; in some embodiments, the nanoparticle has a PDI between about 0.150 and 0.300. In some embodiments, the alkynoate is a C₈ alkynoate; in some embodiments, the nanoparticle has a PDI between about 0.075 and 0.325. In some embodiments, the alkynoate is a C₉ alkynoate; in some embodiments, the nanoparticle has a PDI between about 0.075 and 0.335. In some embodiments, the alkynoate is a C₁₀ alkynoate; in some embodiments, the nanoparticle has a PDI between about 0.225 and 0.310. In some embodiments, the alkynoate is a C₁₁ alkynoate; in some embodiments, the nanoparticle has a PDI between about 0.210 and 0.625. In some embodiments, the alkynoate is a C₁₂ alkynoate; in some embodiments, the nanoparticle has a PDI between about 0.175 and 0.325. In some embodiments, the alkynoate is a C₁₃ alkynoate; in some embodiments, the nanoparticle has a PDI between about 0.050 and 0.410.

In some embodiments, the nanoparticle comprises alkynoate reacted at 5 molar equivalents to PEI. In some embodiments, the alkynoate is a C₆ alkynoate; in some embodiments, the nanoparticle has a PDI between about 0.085 and 0.210. In some embodiments, the alkynoate is a C₇ alkynoate; in some embodiments, the nanoparticle has a PDI between about 0.040 and 0.340. In some embodiments, the alkynoate is a C₈ alkynoate; in some embodiments, the nanoparticle has a PDI between about 0.130 and 0.260. In some embodiments, the alkynoate is a C₉ alkynoate; in some embodiments, the nanoparticle has a PDI between about 0.035 and 0.225. In some embodiments, the alkynoate is a C₁₀ alkynoate; in some embodiments, the nanoparticle has a PDI between about 0.018 and 0.245. In some embodiments, the alkynoate is a C₁₁ alkynoate; in some embodiments, the nanoparticle has a PDI between about 0.135 and 0.225. In some embodiments, the alkynoate is a C₁₂ alkynoate; in some embodiments, the nanoparticle has a PDI between about 0.150 and 0.235. In some embodiments, the alkynoate is a C₁₃ alkynoate; in some embodiments, the nanoparticle has a PDI between about 0.125 and 0.300.

In some embodiments, the nanoparticle comprises alkynoate reacted at 7.5 molar equivalents to PET. In some embodiments, the alkynoate is a C₆ alkynoate; in some embodiments, the nanoparticle has a PDI between about 0.020 and 0.225. In some embodiments, the alkynoate is a C₇ alkynoate; in some embodiments, the nanoparticle has a PDI between about 0.040 and 0.200. In some embodiments, the alkynoate is a C₈ alkynoate; in some embodiments, the nanoparticle has a PDI between about 0.060 and 0.160. In some embodiments, the alkynoate is a C₉ alkynoate; in some embodiments, the nanoparticle has a PDI between about 0.070 and 0.575. In some embodiments, the alkynoate is a C₁₀ alkynoate; in some embodiments, the nanoparticle has a PDI between about 0.130 and 0.210. In some embodiments, the alkynoate is a C₁₁ alkynoate; in some embodiments, the nanoparticle has a PDI between about 0.135 and 0.425. In some embodiments, the alkynoate is a C₁₂ alkynoate; in some embodiments, the nanoparticle has a PDI between about 0.210 and 0.300. In some embodiments, the alkynoate is a C₁₃ alkynoate; in some embodiments, the nanoparticle has a PDI between about 0.225 and 0.280.

In some embodiments, the nanoparticle comprises alkynoate reacted at 10 molar equivalents to PEI. In some embodiments, the alkynoate is a C₆ alkynoate; in some embodiments, the nanoparticle has a PDI between about 0.030 and 0.120. In some embodiments, the alkynoate is a C₇ alkynoate; in some embodiments, the nanoparticle has a PDI between about 0.040 and 0.115. In some embodiments, the alkynoate is a C₈ alkynoate; in some embodiments, the nanoparticle has a PDI between about 0.015 and 0.170. In some embodiments, the alkynoate is a C₉ alkynoate; in some embodiments, the nanoparticle has a PDI between about 0.100 and 0.165. In some embodiments, the alkynoate is a C₁₀ alkynoate; in some embodiments, the nanoparticle has a PDI between about 0.165 and 0.250. In some embodiments, the alkynoate is a C₁₁ alkynoate; in some embodiments, the nanoparticle has a PDI between about 0.095 and 0.250. In some embodiments, the alkynoate is a C₁₂ alkynoate; in some embodiments, the nanoparticle has a PDI between about 0.285 and 0.500. In some embodiments, the alkynoate is a C₁₃ alkynoate; in some embodiments, the nanoparticle has a PDI between about 0.285 and 0.480.

The nanoparticle can further comprise PEG. Total PEG percentage of the formulation for the composition can range from 0.025 mol % to 30 mol %, 0.1 mol % to 20 mol %, 0.5 mol % to 10 mol %, or about 0.025 mol %, 0.05 mol %, 0.075 mol %, 0.1 mol %, 0.2 mol %, 0.3 mol %, 0.4 mol %, 0.5 mol %, 0.6 mol %, 0.7 mol %, 0.8 mol %, 0.9 mol %, 1 mol %, 2 mol %, 3 mol %, 4 mol %, 5 mol %, 6 mol %, 7 mol %, 8 mol %, 9 mol %, 10 mol %, 11 mol %, 12 mol %, 13 mol %, 14 mol %, 15 mol %, 16 mol %, 17 mol %, 18 mol %, 19 mol %, 20 mol %, %, 21 mol %, 22 mol %, 23 mol %, 24 mol %, 25 mol %, 26 mol %, 27 mol %, 28 mol %, 29 mol %, or 20 mol % or any range therein. In an example embodiment, the PEG can be provided at 0.01% to 10.0% by weight, e.g., 0.05% to 5% by weight, 0.1% to 2% by weight, 0.2% to 0.5% by weight. In an example embodiment, the PEG is provided at 0.25%. PEG molecules are known in the art and are commercially available and include PEG groups with different head groups, for example, 1,2-dimyristoyl-rac-glycero-3-methoxypolyethylene glycol-2000 (DMG PEG 2000), ALC-0159 (CAS No. 1849616-42-7), C12, C14, C16, C18 PEG lipids and other PEG lipid conjugates. Exemplary PEG molecular weight can range from about 100 g/mol to about 40,000 g/mole, e.g., 250 g/mol to 20,000 g/mol, 500 g/mol to 15,000 g/mol, 750 g/mol to 10,000 g/mol, 500 g/mol to 8,000 g/mol, or any value in the ranges.

Methods of making the nanoparticle are provided. The making of a nanoparticle can comprise adding a cargo to the functionalized PEI compound, wherein the nanoparticle self-assembles via electrostatic interactions between the cargo and the compound.

Methods of delivering a cargo to a target cell are also provided. The methods comprise contacting the target cell with the nanoparticles detailed herein. The contacting can occur in vivo, ex vivo, or in vitro. In an aspect, the target cell is a liver cell, heart cell, reproductive organ cell, kidney cell, lung cell or spleen cell. In an aspect, the target cell is a lung cell or a spleen cell. In some embodiments, the target cell is a lung cell and the LNP preferentially accumulates and/or targets lung tissue.

Methods of delivering a cargo in a tissue specific manner can comprise contacting the tissue with the nanoparticle described herein. In an embodiment, the nanoparticle has a preference for lung tissue, spleen tissue, liver tissue, heart tissue, reproductive organs, or kidney tissue, that is, targets the preferred tissue at more than 10%, 20%, 30%, 40%, 50% 60%, 70%, 80%, 90%, or more relative to selectivity to other tissues.

Another aspect of the invention is a method of treating a disease in a subject in need thereof, the method comprising administering to the subject a therapeutically effective amount of one or more nanoparticles comprising a cargo, thereby treating the disease.

In some embodiments, the methods treat or prevent a disease, disorder, or condition by introducing a cargo, thereby modulating the expression of a gene. An example application includes vaccine therapies, cancer treatments, infectious disease therapies, and gene editing. The administration of a nanoparticle comprising a cargo may increase or enhance expression or reduce or silence gene expression. Advantageously, in some embodiments, the methods treat a disease, disorder, or condition in the lungs because the LNPs according to some embodiments concentrate in the lungs.

In some embodiments, the methods include vaccinating a subject in need thereof, comprising administering to the subject the nanoparticle as described herein, wherein the cargo provides an immunostimulatory effect.

Methods of detection can be in vitro and can comprise obtaining a sample from the subject. Suitable samples may comprise blood, serum, plasma, brain homogenate, interstitial fluid, cerebral spinal fluid, and/or exocrine gland secretion, and enriched forms thereof. According to one embodiment, the sample is a biological sample from a subject in need thereof. The biological sample can be from a subject that requires diagnosis of disease, and/or monitoring of the effectiveness of a treatment.

Methods of detection can be in vivo and can comprise administering a nanoparticle comprising an imaging agent or detectable label to the subject. The nanoparticle may comprise a labeled biomarker, which includes nucleic acids, proteins, metabolites, and reaction products thereof. Such biomarkers also encompass the mutations, variants, modifications, fragments, and polymorphisms of said biomarkers. Preferably, the subject requires diagnosis of, or is suspected of having, a disease or disorder, and optionally monitoring of the effectiveness of a treatment or progression of a disease. Accordingly, the nanoparticles comprising a cargo such as a biomarker, label or imaging agent are useful in methods of diagnosing, prognosing and/or staging a disease or disorder in a subject by detecting a first level of expression, activity and/or function of one or more biomarker and comparing the detected level to a control of level wherein a difference in the detected level and the control level indicates a change in the disease or disorder.

An aspect of the present invention is a method of transferring or delivering a cargo to a cell in vitro. The nanoparticle comprising a cargo may be introduced, e.g., by contact, into the cells at the appropriate cargo dosage suitable for the particular target cells. Dosages can vary, depending upon the target cell type and number, and the particular nanoparticle and cargo, and can be determined by those of skill in the art without undue experimentation.

A further aspect of the invention is a method of treating subjects in vivo, comprising administering to a subject nanoparticles comprising one or more cargo, which may be further comprised in a pharmaceutically acceptable carrier, wherein the pharmaceutical composition is administered in a therapeutically effective amount. Administration of the nanoparticles of the present invention to a human subject or an animal in need thereof can be by any means known in the art for administering compounds.

### Cargo

The compounds disclosed herein may be provided with cargo molecules as nanoparticles for delivery. Agents including biologically active agents, imaging agents and therapeutic agents may be utilized as cargo molecules in the present invention. Example cargos may be a nucleic acid, a protein, a complex of a nucleic acid and a protein, a carbohydrate, a lipid, or a small molecule. One or two or more different cargos may be delivered by the delivery particles described herein. The cargo can be selected for the treatment of a disease or disorder, for the detection of a disease or disorder, or for monitoring of a disease or disorder. In an example embodiment, the cargo is an mRNA for delivery to a cell to increase expression of one or more proteins. In an embodiment, the cargo is an siRNA for delivery to a cell to decrease expression or achieve gene silencing of one or more target sequences. The cargo may be an imaging agent or detectable label or may be a molecule comprising an imaging agent or detectable label, that can be delivered to a cell or tissue. In an aspect, the cargo is a genetic modulating agent such as a CRISPR-Cas system comprising a CRISRP-Cas protein and a guide sequence specific for a target sequence that can be utilized for a variety of gene editing applications. Accordingly, the particles comprising cargo will find use in therapeutic, detection, diagnostic, and other applications.

### Imaging Agents

Imaging agents, including reporter probes, can be delivered in the compositions, nanoparticles, and methods of the present invention. The imaging agents and reporter probes may be associated with another molecule, covalently or otherwise, for delivery in the nanoparticle. In an aspect, a reporter probe may be associated with a nucleic acid specific for a target sequence in a cell, that can be delivered to a cell, tissue, or a subject in need thereof.

Imaging agents can include molecules suitable for any imaging modality, including positron emission tomography (PET) and single-photon emission computed tomography (SPECT), magnetic resonance imaging (MR, MRI), ultrasound (US), and Computed Tomography (CT) imaging. Example agents can include magnetic agents including paramagnetic agents, such as gadolinium, and superparamagnetic iron-based agents. See, e.g., Xiao, et al. (2016). MRI contrast agents: Classification and application. Int'l J of Mol Med, 38, 1319-1326; doi:10.3892/ijmm.2016.2744. Other contrast media include iodinated, e.g., tri-iodinated, benzene rings, or colloidal or micronized barium sulfate.

Reporter probes, including fluorescent, radioactive, and other photo-emitting compounds, may also be delivered, including synthetic dye families such as tetramethylindo(di)-carbocyanines, fluorescein, and rhodamines. Biosensors may also be used, including in in vitro detection applications for bacterial, viral, and other clinical applications. See, e.g., Castillo-Henriquez et al., Sensors (Basel). 2020 December; 20(23): 6926; doi: 10.3390/s20236926.

### Biologically Active Agents

A biologically active agent, i.e., an agent that modulates an effect or activity in a cell, tissue, organ, or other biological media such as biological fluid, includes nucleic acids, proteins, small molecules, carbohydrates, lipids and complexes and combinations thereof. The biologically active agent can be a genetic modifying agent. Representative nucleic acids include DNA, RNA, transposon DNA, antisense nucleic acids, ribozymes, plasmids, expression constructs, and RNA, such as mRNA, tRNA, ribosomal RNA, small nucleolar RNA, antisense oligonucleotides, or RNAi, such as siRNA, shRNA, and miRNA.

RNAi therapeutic comprises a polynucleotide that is complementary to a portion of the target sequence mRNA, generally ranging in size from 15 to 50 base pairs. In an example embodiment, the siRNA is a nucleic acid that can form a double stranded RNA with the ability to reduce or inhibit expression of a gene or target gene: each complementary sequence of the double stranded siRNA is about 15-50 nucleotides in length, and the double stranded siRNA is about 15-50 base pairs in length. A small hairpin RNA (shRNA) is also contemplated for use. The shRNA is an antisense strand of about 19 to about 25 nucleotides followed by a short nucleotide loop (approximately 5 to 9 nt) followed by the analogous sense strand. In an embodiment, an RNAi is a microRNA or miRNA, endogenous RNAs, some of which are known to regulate the expression of protein-coding genes at the posttranscriptional level. See, e.g., Lim et al Science 299, 1540 (2003), Lee and Ambros Science, 294, 862 (2001), Lau et al., Science 294, 858-861 (2001), Lagos-Quintana et al, RNA, 9, 175-179 (2003).

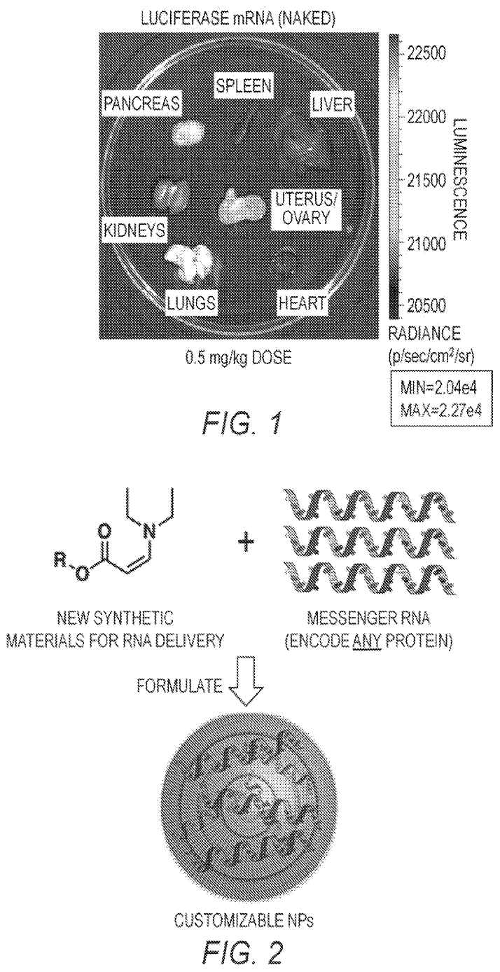

Different criteria are available for selecting the nucleic acid for use and may comprise scanning the mRNA sequence of the target, and may include empiric determination in accordance with, for example, Sui G et al., Proc. Natl. Acad. Sci. USA 99:5515-20 (2002) and may include confirmation the sequence lacks significant sequence homology with other genes as analyzed by BLAST search. Additional approaches may comprise any accessible site in endogenous mRNA that can be targeted for degradation by synthetic oligodeoxyribonucleotide/RNase H method (see, e.g., Lee N S et al., Nature Biotechnol. 20:500-05 (2002)). RNAi treatment may comprise miRNA or siRNA, or a pre-miRNA which is processed by Dicer to form a miRNA. The RNAi may also comprise a dsRNA or shRNA which is processed by Dicer to form a siRNA. The polynucleotides may comprise one or more modifications to suppress innate immune activation, enhance activity and specificity, and reduce off-target induced toxicity. Example teachings can be found, for example at Provost et al., E.M.B.O. J., 2002 Nov. 1; 21(21): 5864-5874; Tabara et al., Cell 2002, June 28; 109(7):861-71; Martinez et al., Cell 2002, September. 6; 110(5):563; Hutvagner & Zamore, Science 2002, 297:2056. In certain embodiments, a single-stranded RNAi agent disclosed herein can comprise substitutions, or modifications, including chemically modified nucleotides, and non-nucleotides which may include incorporation in the backbone, sugars, bases, or nucleosides. In an example, siRNA may comprise dual ribose modifications, including 2′,4′- and 2′,5′-modifications, 5′-E-Z-vinylphosphonate, and northern methanocarbacyclic (NMC) modifications. See, Gangopadhyay, RNA Biol. 2022 January; 19(1):452-467; doi: 10.1080/15476286.2022.2052641. The use of substituted or modified single-stranded RNAi agents can be designed to have an increased half-life in a subject. Furthermore, certain substitutions or modifications can be used to improve the bioavailability of single-stranded RNAi agents by targeting particular cells or tissues or improving cellular uptake of the single-stranded RNAi agents. Exemplary modifications and locations within a RNAi polynucleotide are described in Hu et al. “Therapeutic siRNA: State of the Art” Signal Transduction and Targeted Therapy 5, Article number 100 (2020), incorporated herein by reference, see, e.g., FIGS. 2 and 3, specifically for its teachings of modifications.

The RNAi molecule may decrease the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule.

The cargo may include a gene modulating agent, including gene editing systems. Gene editing systems may comprise a CRISPR system, a zinc finger nuclease system, a meganuclease, or a TALE system. A CRISPR-Cas system can comprise a Class 1 or Class 2 CRISPR-Cas system, which may comprise a guide sequence engineered to specifically bind a polynucleotide of interest. As such, a ribonucleoprotein comprised of a Cas protein and a guide polynucleotide can be delivered. the CRISPR-Cas system that can be used to modify a target polynucleotide of the present invention described herein can be a Class 1 CRISPR-Cas system. Class 1 CRISPR-Cas systems are divided into types I, II, and IV. Makarova et al. 2020. Nat. Rev. 18: 67-83., particularly as described in FIG. 1. Type I CRISPR-Cas systems include Types I-A, I-B, I-C, I-D, I-E, I-F1, I-F2, I-F3, and IG; Type III CRISPR-Cas systems can be Types III-A, III-B, III-C, III-D, III-E, and III-F; which can contain a Cas10 that can include an RNA recognition motif called Palm and a cyclase domain that can cleave polynucleotides; Type IV CRISPR-Cas systems include Types IV-A, IV-B, and IV-C. Class 2 systems comprise a single, large, multi-domain effector protein and can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Class 2, Type II systems include II-A, II-B, II-C1, and II-C2; Type V systems include V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type VI systems include VI-A, VI-B1, VI-B2, VI-C, and VI-D. Design of guides for targeting a nucleic acid for modification is known in the art, see, e.g., IDTdna.com and Synthego.com for guidance on custom guide RNAs. Reduction of off-target effects can be tailored using programs such as GUIDE-seq for the design of guide sequences for a desired target. See, e.g., Malinin, et al., Nature Protocols, 16, 5592-5615 (2012). TALEN based gene editing is also contemplated and can be used in in vivo applications. See, S Becker, J Boch—Gene and Genome Editing, 2021. Zinc finger nuclease editing can also be utilized, and further modified to ensure high-precision gene editing. See, e.g., Conway et al., Molecular Therapy, 27:4, 10 Apr. 2019, Pages 866-877; Paschon et al. Nature Comm, 10:1133 (2019). Similarly, editing can be made by meganucleases, characterized by a large recognition site of 12 to 40 based pairs of a double-stranded DNA sequence. See, e.g., U.S. Pat. Nos. 8,119,381, 10,273,524. Gene editing tools are well known in the art, with advantages and comparison of the tools that can be considered for the desired application. Rahim et al., Int'l J. of Innovative Science and Research Tech., 6:8 (2021), incorporated herein by reference.

### Transposase

Transposases may be used with the methods of the present invention. Transposases include those comprising RNase H-like nuclease domains, such as Tn5, MuA, Mos1, Hermes, Serine and Tyrosine recombinases, including CTnDOT, Tn916, IS607 and TnpX, transposases comprising an HUH domain, including TnpA of IS91 or ISHp608, and helitron transposases, which can be as detailed in International Patent Publication WO2022056309, page 26, line 26-page 27, line 17, specifically incorporated by reference. See also nuclease guided transposase as described in WO2022150651 (DNA nuclease guided Transposases systems, Tn7-like transposition proteins with a Cas12 k protein), WO2022147321 (Type I-B CRISPR Associated Transposase systems), WO2022076830 (Type I CRISPR Associated transposase systems), WO2021257997 (CAST); Li, et al., Int. J. Mol. Sci. 2020, 21(21), 8329; doi: 10.3390/ijms21218329 (Tn5 transposase in applied genomic research).

### Therapeutic Agents

Therapeutic agents that can be used as cargo with the nanoparticles can comprise modulating agents, for example, small molecules such as chemotherapeutic agents, anti-oncogenic agents, anti-microbial agents, peptides, proteins (enzymes, antibodies, peptidic hormones), non-peptidic hormones, other pharmaceutically active substances, and the like.

Examples of the protein or peptide can include hormones such as growth hormones, growth factors, cytokines, tumor necrosis factors, growth hormone releasing factors, steroid sparing agents such as cyclosporine, thyroid hormones, adrenaline, insulin, cortisol, estrogen and progesterone. Example enzymes can include transferases, hydrolases, lyases, isomerases, ligases, oxidoreductases, and translocases.

Anti-microbials such as antibiotics, antivirals, antifungals and antiparasitics can be delivered in the nanoparticles of the present invention. Accordingly, the antimicrobials can be used to prevent and/or treat infections in a subject.

Examples of a chemotherapeutic agent include without limitation: alkylating agents (e.g., which may include doxorubicin, cyclophosphamide, estramustine, carmustine, mitomycin, bleomycin and the like); antimetabolites (e.g., which may include 5-fluoro-uracil, capecitabine, gemcitabine, nelarabine, fludarabine, methotrexate and the like); platinating agents (e.g., which may include cisplatin, oxaliplatin, carboplatin and the like); topoisomerase inhibitors (e.g., which may include topotecan, irinotecan, etoposide and the like); tubulin agents (e.g., which may include paclitaxel, docetaxel, vinorelbine, vinblastine, vincristine, other taxanes, epothilones, and the like); signaling inhibitors (e.g., kinase inhibitors, antibodies, farnesyltransferase inhibitors, and the like); and other chemotherapeutic agents (e.g., tamoxifen, anti-mitotic agents such as polo-like kinase inhibitors or aurora kinase inhibitors, and the like).

Antibodies can be utilized in the methods of the invention. An antibody, or antigen binding fragment thereof, including polyclonal and monoclonal antibodies can be utilized as cargo. The term “antibody fragment” refers to a portion of an immunoglobulin, often the hypervariable region and portions of the surrounding heavy and light chains that displays specific binding affinity for a particular target, typically a molecule. A hypervariable region is a portion of an immunoglobulin that physically binds to the polypeptide target. An antibody fragment thus includes or consists of one or more portions of a full-length immunoglobulin retaining the targeting specificity of the immunoglobulin. Such antibody fragment may for instance lack at least partially the constant region (Fc region) of the full-length immunoglobulin. In some embodiments, an antibody fragment is produced by digestion of the full-length immunoglobulin. An antibody fragment may also be a synthetic or recombinant construct that contains one or more parts of the immunoglobulin or immunoglobulin chains (see e.g., HOLLIGER, P. and Hudson, J. Engineered antibody fragments and the rise of single domains. Nature Biotechnology 2005, vol. 23, no. 9, p. 1126-1136). Examples of an antibody fragment include, but are not limited to, an scFv, a Fab, a Fv, a Fab′, a F(ab′)2 fragment, a dAb, a VHH, a nanobody, a V(NAR) or a minimal recognition unit.

Non-limiting examples of formulations of the invention include those suitable for intravenous administration of the nanoparticles. Oral, rectal, buccal (e.g., sub-lingual), vaginal, parenteral (e.g., subcutaneous, intramuscular including skeletal muscle, cardiac muscle, diaphragm muscle and smooth muscle, intradermal, intravenous, intraperitoneal), topical (i.e., both skin and mucosal surfaces, including airway surfaces), intranasal, transdermal, intraarticular, intracranial, intrathecal, and inhalation administration, administration to the liver by intraportal delivery, as well as direct organ injection (e.g., into the liver, into a limb, into the brain or spinal cord for delivery to the central nervous system, into the pancreas, or into a tumor or the tissue surrounding a tumor) are also envisioned. The most suitable route in any given case will depend on the nature and severity of the condition being treated and on the nature of the particular nanoparticle which is being used. In some embodiments, it may be desirable to deliver the formulation locally to avoid any side effects associated with systemic administration. For example, local administration can be accomplished by direct injection at the desired treatment site, by introduction intravenously at a site near a desired treatment site (e.g., into a vessel that feeds a treatment site, or intramuscular administration with muscle specific promoters). In some embodiments, the formulation can be delivered locally to ischemic tissue.

For injection, the carrier will typically be a liquid, such as sterile pyrogen-free water, pyrogen-free phosphate-buffered saline solution, bacteriostatic water, or Cremophor EL[R] (BASF, Parsippany, N.J.). For other methods of administration, the carrier can be either solid or liquid.

For oral administration, the compound can be administered in solid dosage forms, such as capsules, tablets, and powders, or in liquid dosage forms, such as elixirs, syrups, and suspensions. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Formulations suitable for buccal (sub-lingual) administration include lozenges comprising the compound in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the compound, which preparations are preferably isotonic with the blood of the intended recipient. These preparations can contain anti-oxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

In one aspect of the present invention, there is provided an injectable, stable, sterile composition comprising a nanoparticle of the invention, in a unit dosage form in a sealed container. The nanoparticle is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the nanoparticle. When the nanoparticle is substantially water-insoluble, a sufficient amount of emulsifying agent which is pharmaceutically acceptable can be employed in sufficient quantity to emulsify the nanoparticle in an aqueous carrier. One such useful emulsifying agent is phosphatidyl choline.

Formulations suitable for rectal administration are preferably presented as unit dose suppositories. These can be prepared by admixing the nanoparticle with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which can be used include petroleum jelly, lanoline, polyethylene glycols, alcohols, transdermal enhancers, and combinations of two or more thereof.

Formulations suitable for transdermal administration can be presented as discrete patches adapted to remain in intimate contact with the epidermis of the recipient for a prolonged period of time. Formulations suitable for transdermal administration can also be delivered by iontophoresis (see, for example, Tyle, Pharm. Res. 3:318 (1986)) and typically take the form of an optionally buffered aqueous solution of the nanoparticle. Suitable formulations comprise citrate or bis\tris buffer (pH 6) or ethanol/water and contain from 0.1 to 0.2M of the nanoparticle.

The nanoparticle can alternatively be formulated for nasal administration or otherwise administered to the lungs of a subject by any suitable means, e.g., administered by an aerosol suspension of respirable particles comprising the compound, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. See, Lokugamage, et al., Optimization of lipid nanoparticles for the delivery of nebulized therapeutic mRNA to the lungs, Nature, Biomed Eng., 5, 1059-1068 (2021). Aerosol also includes a dry powder composition suspended in air or other carrier gas, which can be delivered by insufflation from an inhaler device, for example. See Ganderton & Jones, Drug Delivery to the Respiratory Tract, Ellis Horwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the nanoparticle can be produced by any suitable means, such as with a pressure-driven aerosol nebulizer or an ultrasonic nebulizer, as is known to those of skill in the art. See, e.g., U.S. Pat. No. 4,501,729. Aerosols of the particles of the invention can likewise be produced with any solid particulate medicament aerosol generator, by techniques known in the pharmaceutical art.

Having described the present invention, the same will be explained in greater detail in the following examples, which are included herein for illustration purposes only, and which are not intended to be limiting to the invention.

### EXAMPLES

### Example 1. Approach for Functionalized PEI Compounds

mRNA injected alone does not express in mouse organs (FIG. 1). Other cargos face challenges in delivery into cells as well as subsequent use and release in cells. FIG. 2 provides an example approach to formulate customizable nanoparticles (NP) utilizing synthetic materials for RNA and other cargo delivery. The synthesis scheme for a compound library of PEI functionalized with C₆-C₁₈ alkynoates is shown in FIG. 3.

### Example 2. Synthesis and Analysis of Compounds

Synthetic Procedure for 2.5C₆. To PEI (36 mg, 0.06 mol, 1 eq) in dichloromethane (875 μL) was added a solution of C₆ alkynoate (23.136 mg of alkynoate in 125 μL of dichloromethane, 0.15 mmol, 2.5 eq). The mixture was stirred for 72 h at room temperature and was then concentrated under a high-pressure vacuum to yield 2.5C₆.

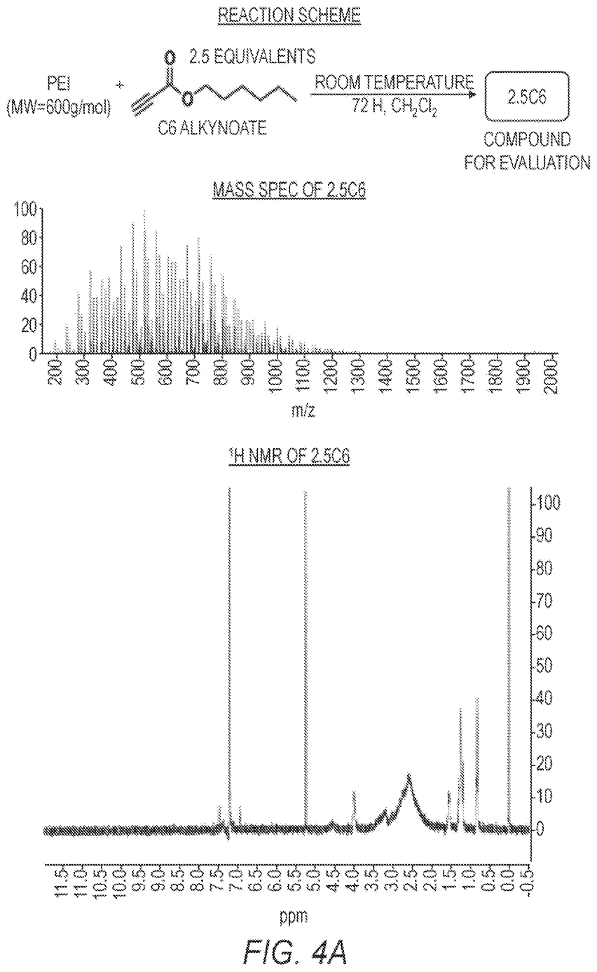

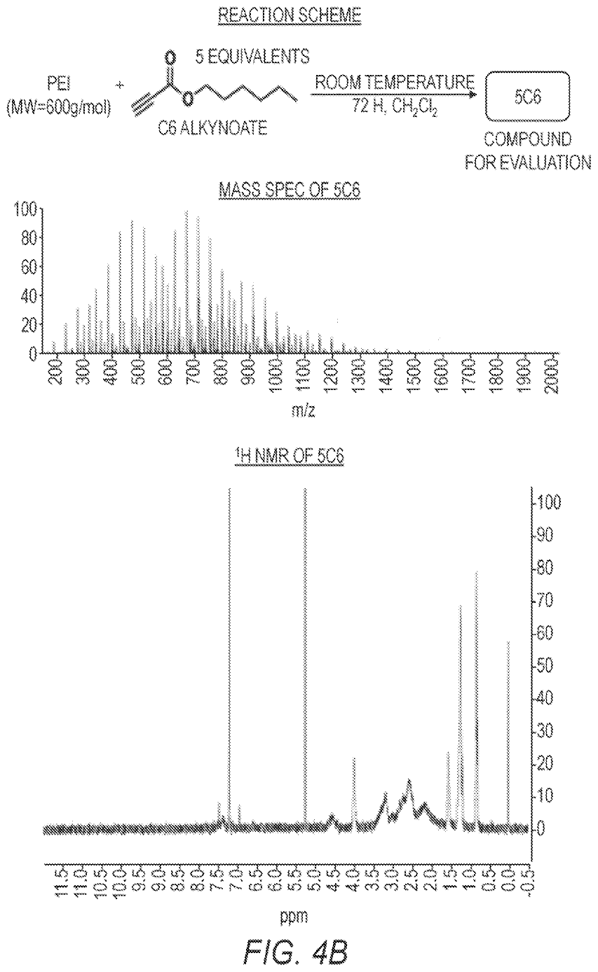

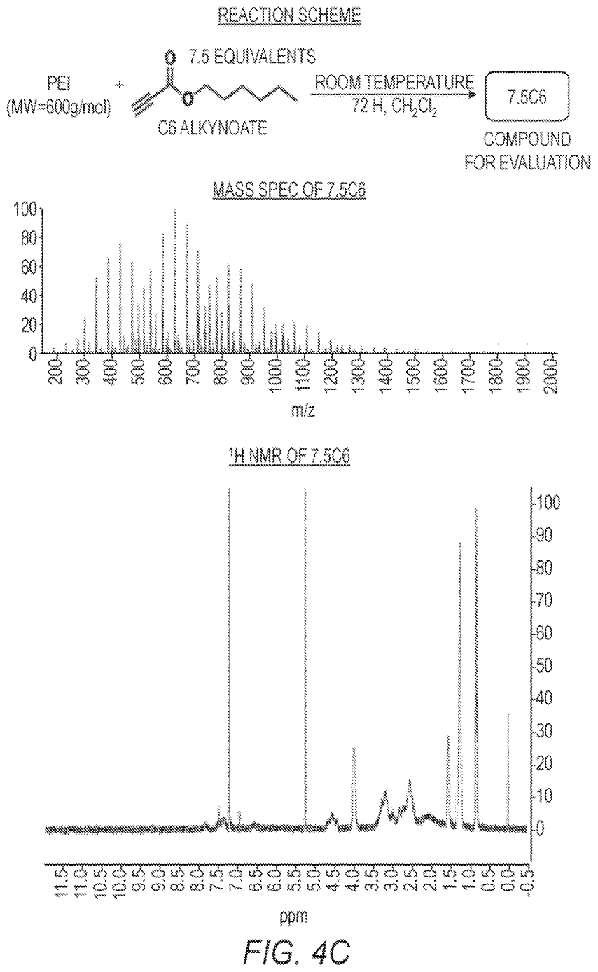

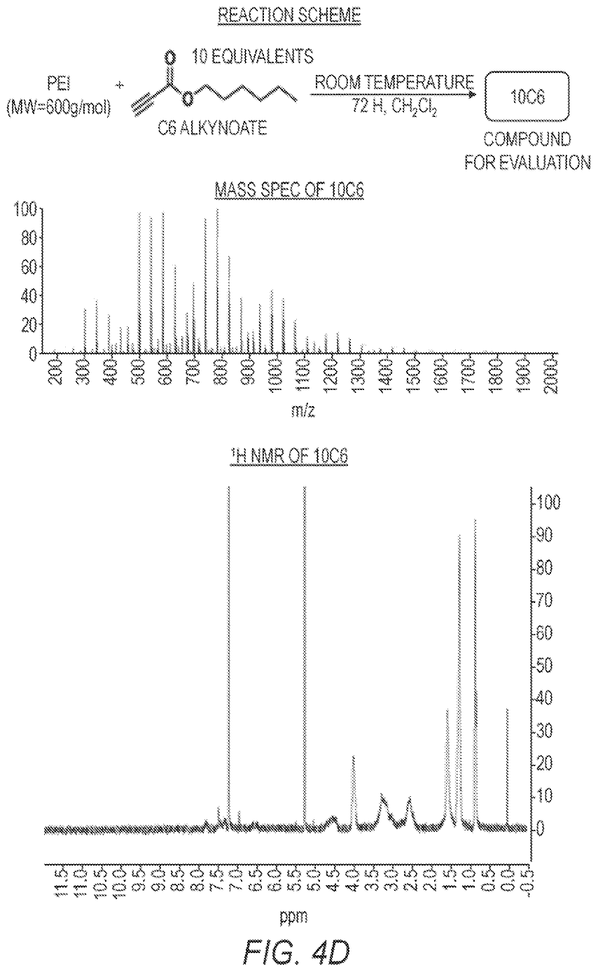

Reaction Scheme, Mass Spectrometry Data and ¹H NMR of PEI compound functionalized with 2.5C₆ is shown in FIG. 4A.

Synthetic Procedure for 5C₆. To PET (36 mg, 0.06 mol, 1 eq) in dichloromethane (750 μL) was added a solution of C₆ alkynoate (46.263 mg of alkynoate in 250 μL of dichloromethane, 0.3 mmol, 5 eq). The mixture was stirred for 72 h at room temperature and was then concentrated under a high-pressure vacuum to yield 5C₆.

Reaction Scheme, Mass Spectrometry Data and ¹H NMR of PEI compound functionalized with 5C₆ is shown in FIG. 4B.

Synthetic Procedure for 7.5C₆. To PEI (36 mg, 0.06 mol, 1 eq) in dichloromethane (625 μL) was added a solution of C₆ alkynoate (69.395 mg of alkynoate in 375 μL of dichloromethane, 0.45 mmol, 7.5 eq). The mixture was stirred for 72 h at room temperature and was then concentrated under a high-pressure vacuum to yield 7.5C₆.

Reaction Scheme, Mass Spectrometry Data and ¹H NMR of PEI compound functionalized with 7.5C₆ is shown in FIG. 4C.

Synthetic Procedure for 10C₆. To PEI (36 mg, 0.06 mol, 1 eq) in dichloromethane (500 μL) was added a solution of C₆ alkynoate (92.526 mg of alkynoate in 500 μL of dichloromethane, 0.6 mmol, 10 eq). The mixture was stirred for 72 h at room temperature and was then concentrated under a high-pressure vacuum to yield 10C₆.

Reaction Scheme, Mass Spectrometry Data and ¹H NMR of PEI compound functionalized with 10C₆ is shown in FIG. 4D.

Synthetic Procedure for 2.5 C₇. To PEI (36 mg, 0.06 mol, 1 eq) in dichloromethane (875 μL) was added a solution of C₇ alkynoate (25.236 mg of alkynoate in 125 μL of dichloromethane, 0.15 mmol, 2.5 eq). The mixture was stirred for 72 h at room temperature and was then concentrated under a high-pressure vacuum to yield 2.5C₇.

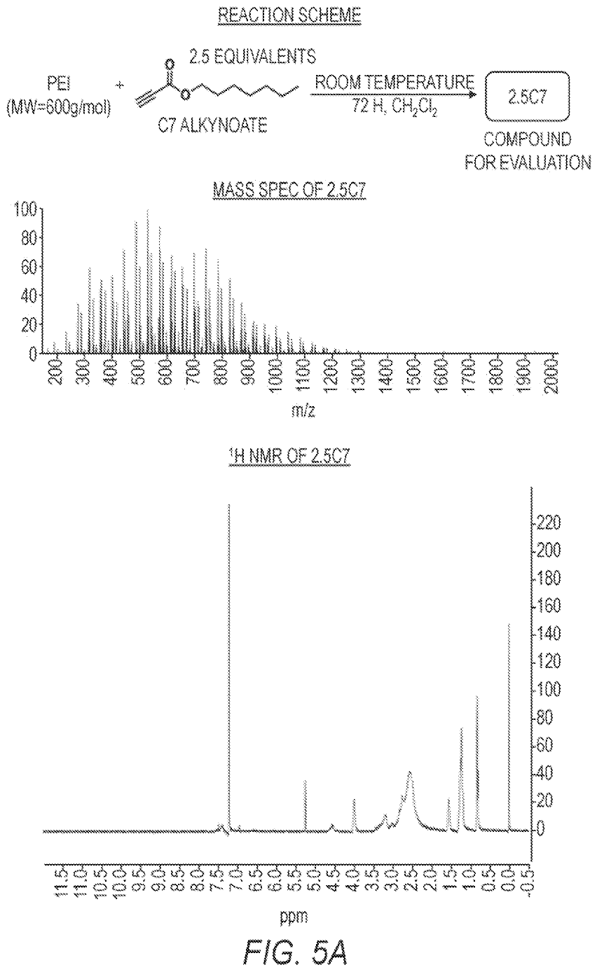

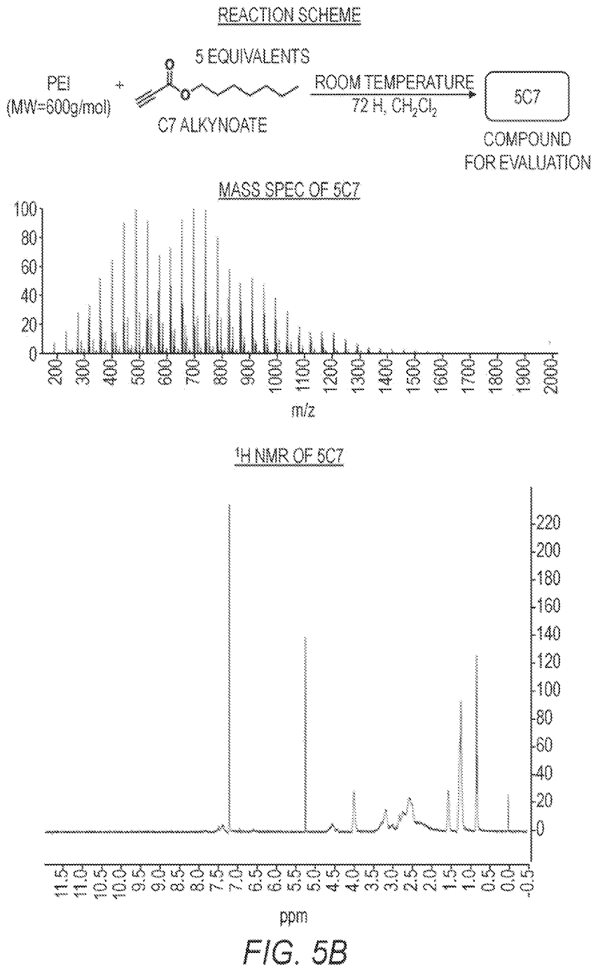

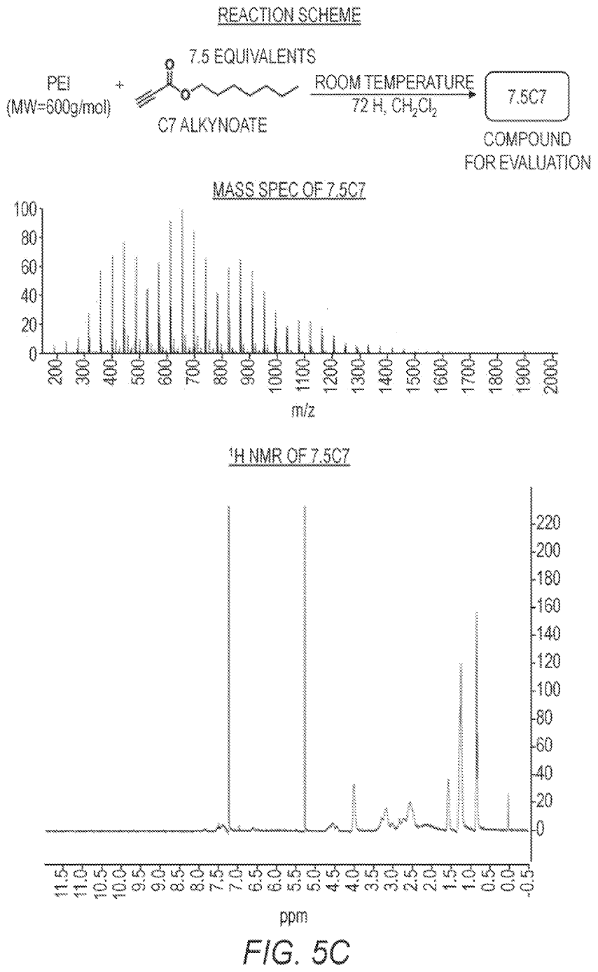

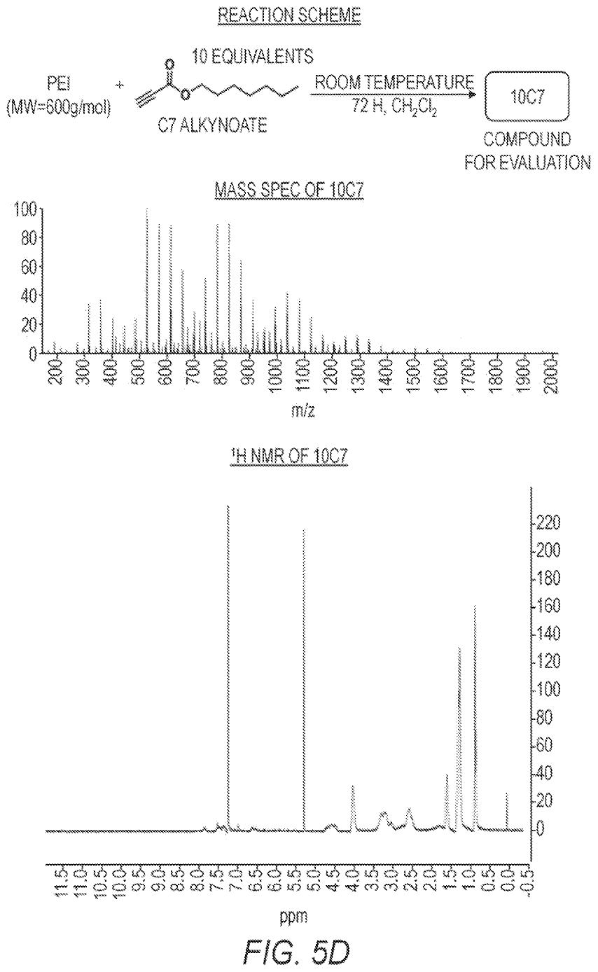

Reaction Scheme, Mass Spectrometry Data and ¹H NMR of PEI compound functionalized with 2.5C₇ is shown in FIG. 5A.

Synthetic Procedure for 5C₇. To PEI (36 mg, 0.06 mol, 1 eq) in dichloromethane (750 μL) was added a solution of C₇ alkynoate (50.472 mg of alkynoate in 250 μL of dichloromethane, 0.3 mmol, 5 eq). The mixture was stirred for 72 h at room temperature and was then concentrated under a high-pressure vacuum to yield 5C₇.

Reaction Scheme, Mass Spectrometry Data and ¹H NMR of PEI compound functionalized with 5C₇ is shown in FIG. 5B.

Synthetic Procedure for 7.5C₇. To PEI (36 mg, 0.06 mol, 1 eq) in dichloromethane (625 μL) was added a solution of C₇ alkynoate (75.708 mg of alkynoate in 375 μL of dichloromethane, 0.45 mmol, 7.5 eq). The mixture was stirred for 72 h at room temperature and was then concentrated under a high-pressure vacuum to yield 7.5C₇.

Reaction Scheme, Mass Spectrometry Data and ¹H NMR of PEI compound functionalized with 7.5C₇ is shown in FIG. 5C.

Synthetic Procedure for 10C₇. To PEI (36 mg, 0.06 mol, 1 eq) in dichloromethane (500 μL) was added a solution of C₇ alkynoate (100.944 mg of alkynoate in 500 μL of dichloromethane, 0.6 mmol, 10 eq). The mixture was stirred for 72 h at room temperature and was then concentrated under a high-pressure vacuum to yield 10C₇.

Reaction Scheme, Mass Spectrometry Data and ¹H NMR of PEI compound functionalized with 10C₇ is shown in FIG. 5D.

Synthetic Procedure for 2.5C₈. To PEI (36 mg, 0.06 mol, 1 eq) in dichloromethane (875 μL) was added a solution of C₈ alkynoate (27.339 mg of alkynoate in 125 μL of dichloromethane, 0.15 mmol, 2.5 eq). The mixture was stirred for 72 h at room temperature and was then concentrated under a high-pressure vacuum to yield 2.5C₈.

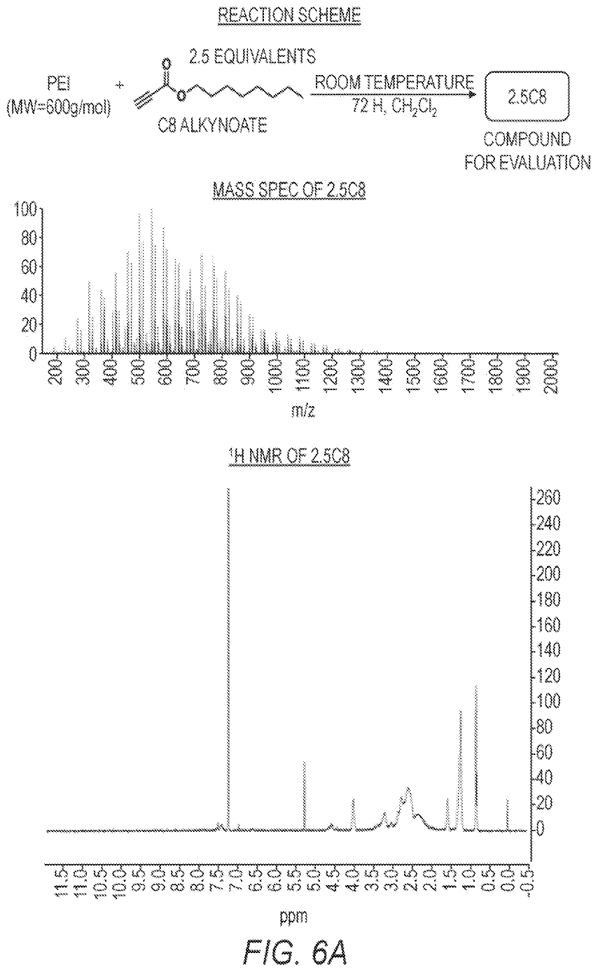

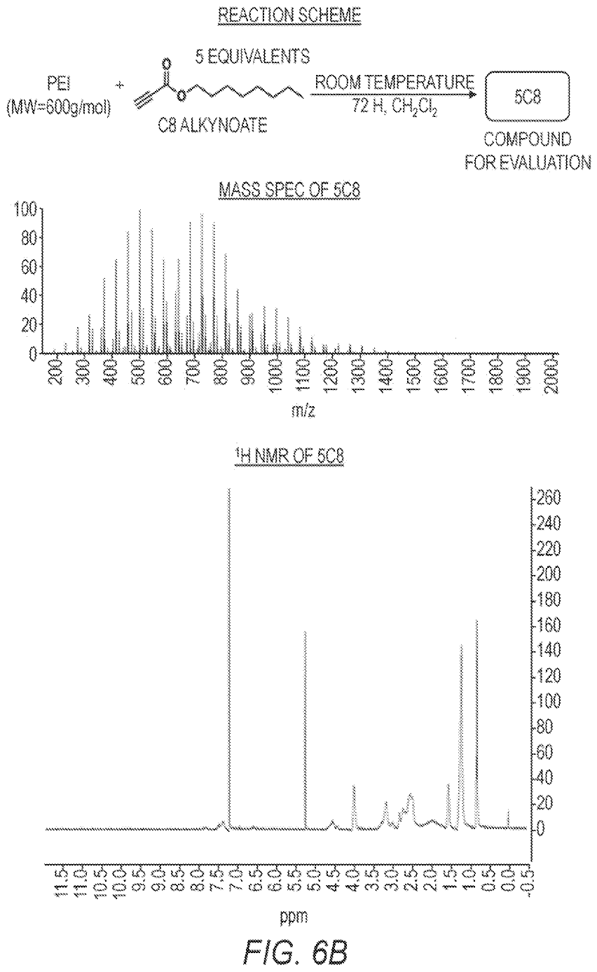

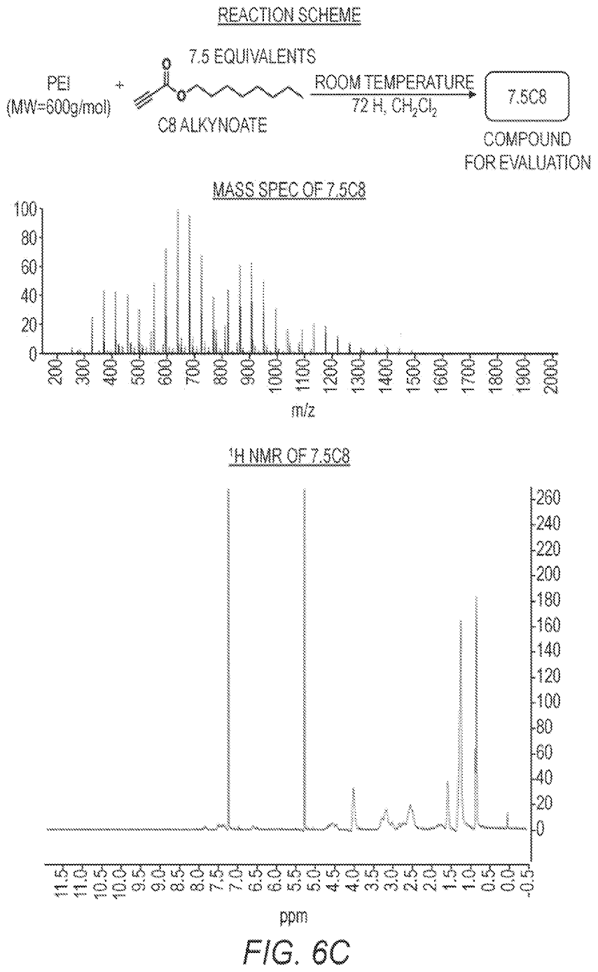

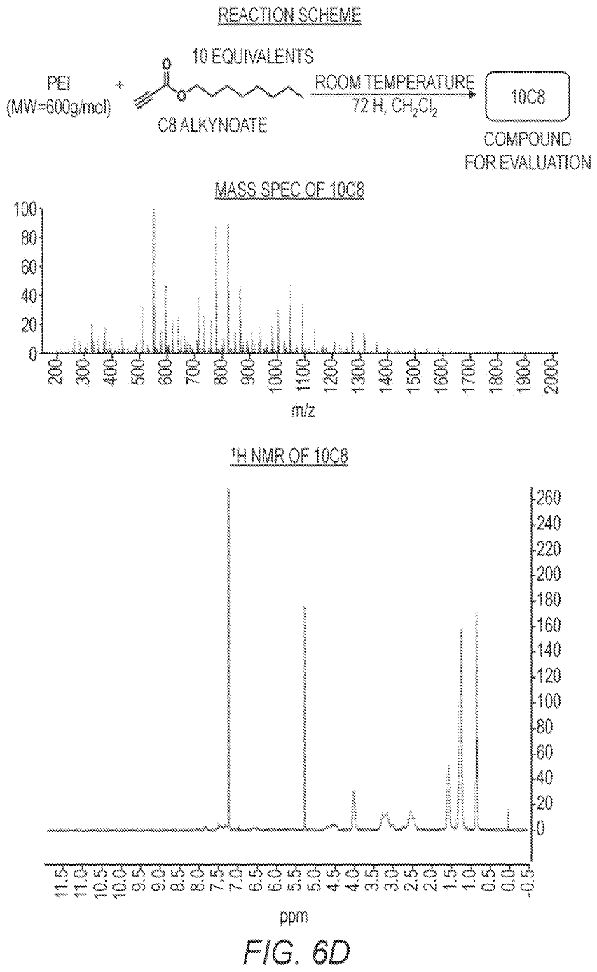

Reaction Scheme, Mass Spectrometry Data and ¹H NMR of PEI compound functionalized with 2.5C₈ is shown in FIG. 6A.

Synthetic Procedure for 5C₈. To PEI (36 mg, 0.06 mol, 1 eq) in dichloromethane (750 μL) was added a solution of C₈ alkynoate (54.678 mg of alkynoate in 250 μL of dichloromethane, 0.3 mmol, 5 eq). The mixture was stirred for 72 h at room temperature and was then concentrated under a high-pressure vacuum to yield 5C₈.

Reaction Scheme, Mass Spectrometry Data and ¹H NMR of PEI compound functionalized with 5C₈ is shown in FIG. 6B.

Synthetic Procedure for 7.5C₈. To PEI (36 mg, 0.06 mol, 1 eq) in dichloromethane (625 μL) was added a solution of C₈ alkynoate (82.017 mg of alkynoate in 375 μL of dichloromethane, 0.45 mmol, 7.5 eq). The mixture was stirred for 72 h at room temperature and was then concentrated under a high-pressure vacuum to yield 7.5C₈.

Reaction Scheme, Mass Spectrometry Data and ¹H NMR of PEI compound functionalized with 7.5C₈ is shown in FIG. 6C.

Synthetic Procedure for 10C₈. To PEI (36 mg, 0.06 mol, 1 eq) in dichloromethane (500 μL) was added a solution of C₈ alkynoate (109.356 mg of alkynoate in 500 μL of dichloromethane, 0.6 mmol, 10 eq). The mixture was stirred for 72 h at room temperature and was then concentrated under a high-pressure vacuum to yield 10C₈.

Reaction Scheme, Mass Spectrometry Data and ¹H NMR of PEI compound functionalized with 10C₈ is shown in FIG. 6D.

Synthetic Procedure for 2.5C₉. To PEI (36 mg, 0.06 mol, 1 eq) in dichloromethane (875 μL) was added a solution of C₉ alkynoate (29.444 mg of alkynoate in 125 μL of dichloromethane, 0.15 mmol, 2.5 eq). The mixture was stirred for 72 h at room temperature and was then concentrated under a high-pressure vacuum to yield 2.5C₉.

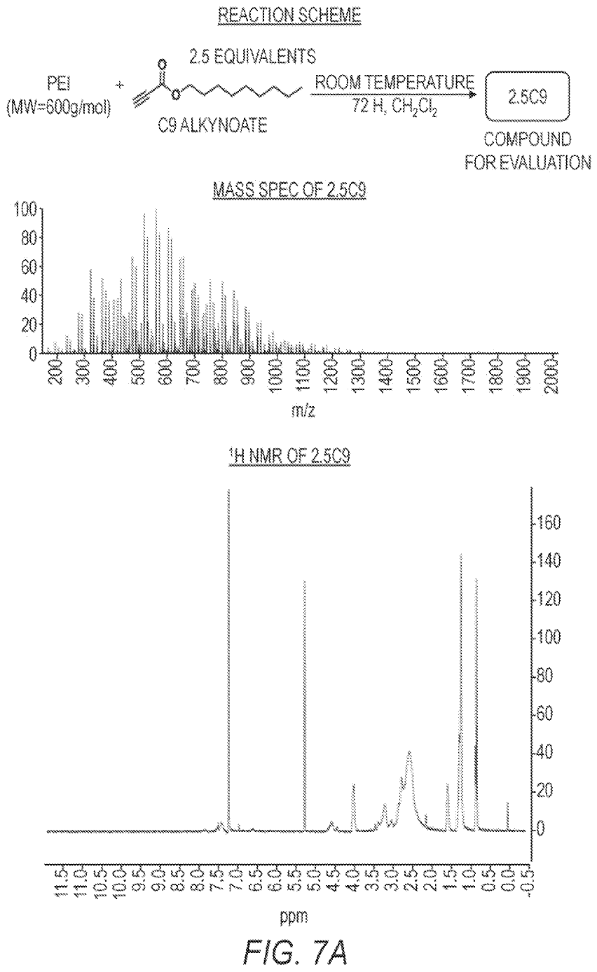

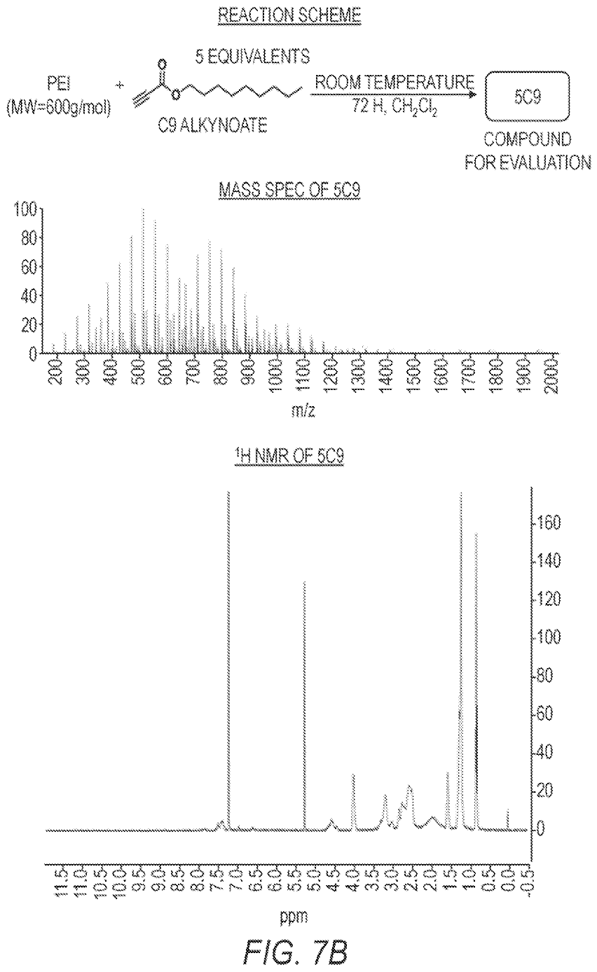

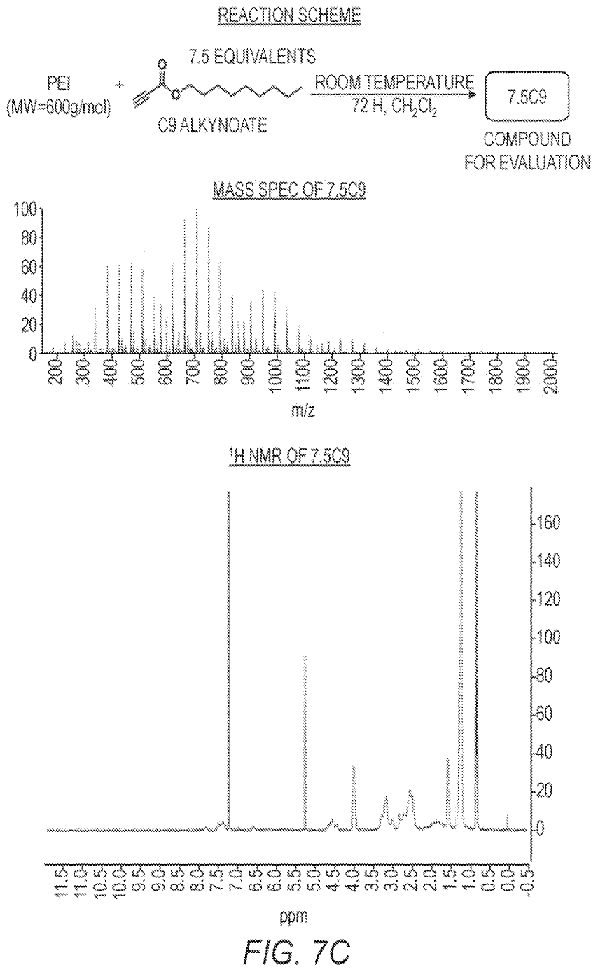

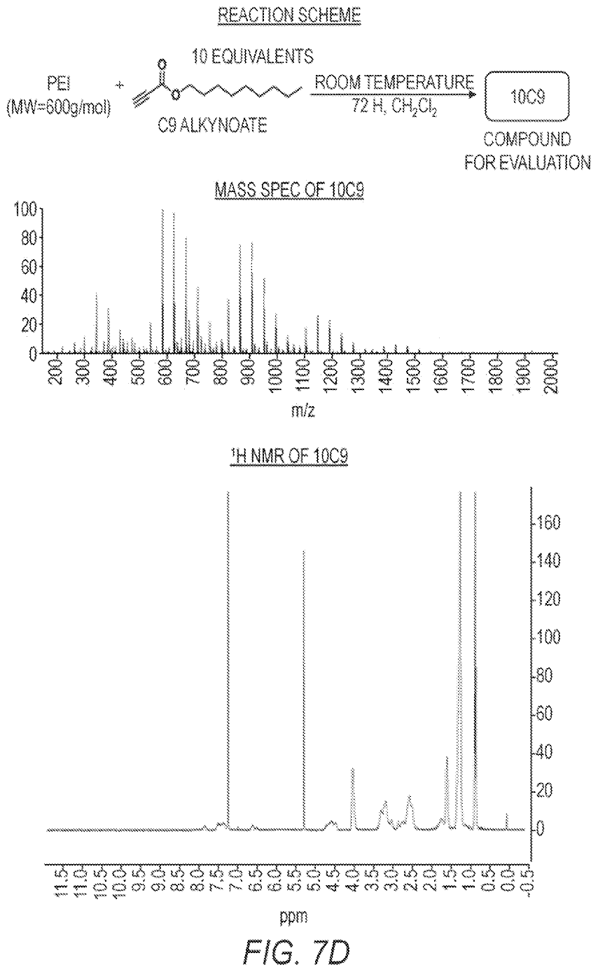

Reaction Scheme, Mass Spectrometry Data and ¹H NMR of PEI compound functionalized with 2.5C₉ is shown in FIG. 7A.

Synthetic Procedure for 5C₉. To PEI (36 mg, 0.06 mol, 1 eq) in dichloromethane (750 μL) was added a solution of C₉ alkynoate (58.887 mg of alkynoate in 250 μL of dichloromethane, 0.3 mmol, 5 eq). The mixture was stirred for 72 h at room temperature and was then concentrated under a high-pressure vacuum to yield 5C₉.

Reaction Scheme, Mass Spectrometry Data and ¹H NMR of PEI compound functionalized with 5C₉ is shown in FIG. 7B.

Synthetic Procedure for 7.5C₉. To PEI (36 mg, 0.06 mol, 1 eq) in dichloromethane (625 μL) was added a solution of C₉ alkynoate (88.331 mg of alkynoate in 375 μL of dichloromethane, 0.45 mmol, 7.5 eq). The mixture was stirred for 72 h at room temperature and was then concentrated under a high-pressure vacuum to yield 7.5C₉.

Reaction Scheme, Mass Spectrometry Data and ¹H NMR of PEI compound functionalized with 7.5C₉ is shown in FIG. 7C.

Synthetic Procedure for 10C₉. To PEI (36 mg, 0.06 mol, 1 eq) in dichloromethane (500 μL) was added a solution of C₉ alkynoate (117.774 mg of alkynoate in 500 μL of dichloromethane, 0.6 mmol, 10 eq). The mixture was stirred for 72 h at room temperature and was then concentrated under a high-pressure vacuum to yield 10C₉.

Reaction Scheme, Mass Spectrometry Data and ¹H NMR of PEI compound functionalized with 10C₉ is shown in FIG. 7D.

Synthetic Procedure for 2.5C₁₀. To PEI (36 mg, 0.06 mol, 1 eq) in dichloromethane (875 μL) was added a solution of C₁₀ alkynoate (31.548 mg of alkynoate in 125 μL of dichloromethane, 0.15 mmol, 2.5 eq). The mixture was stirred for 72 h at room temperature and was then concentrated under a high-pressure vacuum to yield 2.5C₁₀.

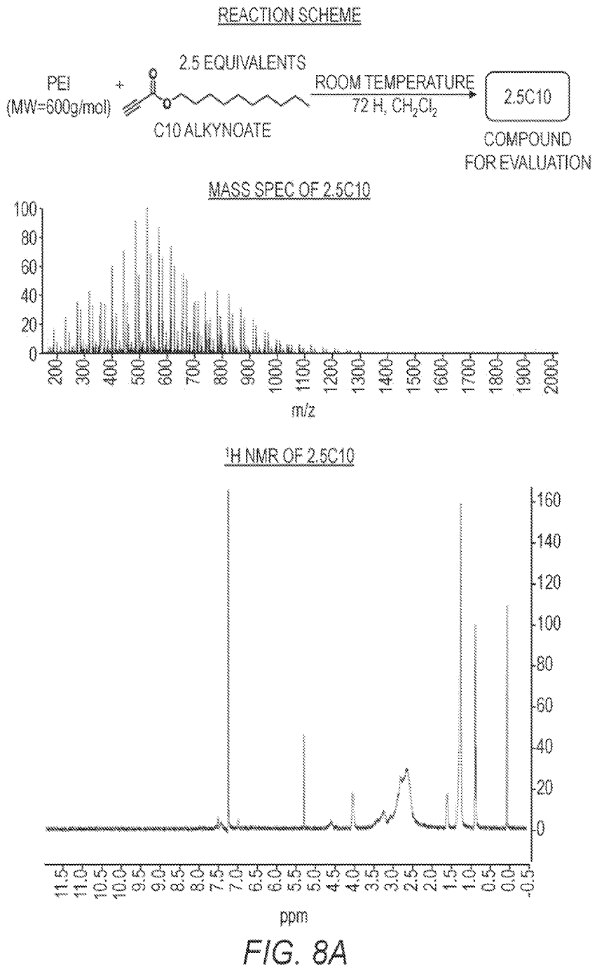

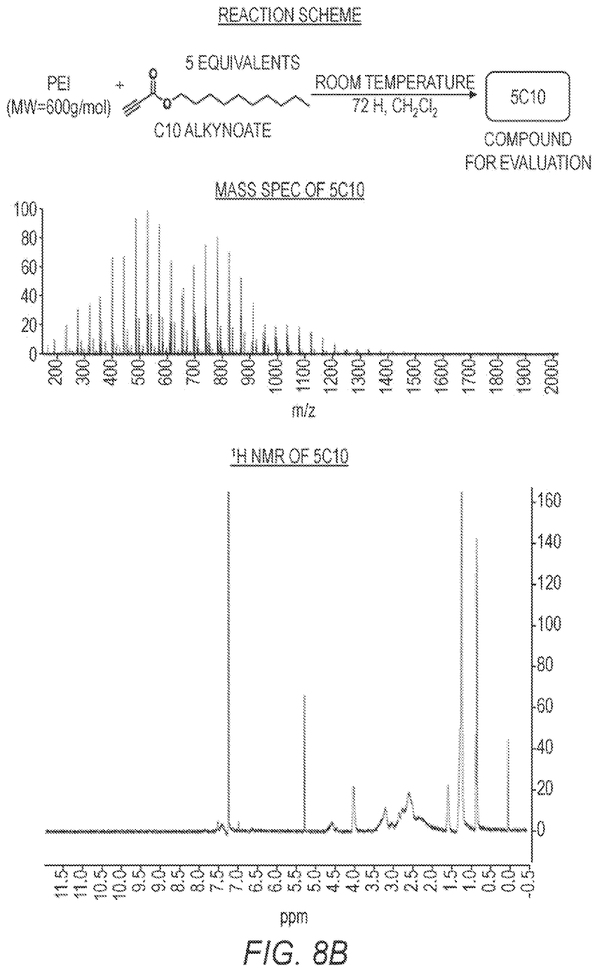

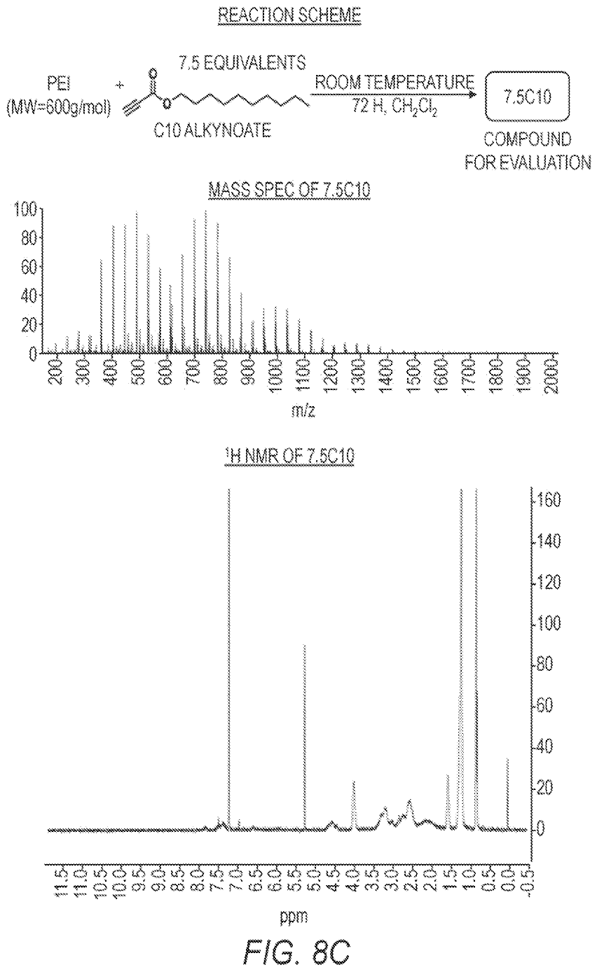

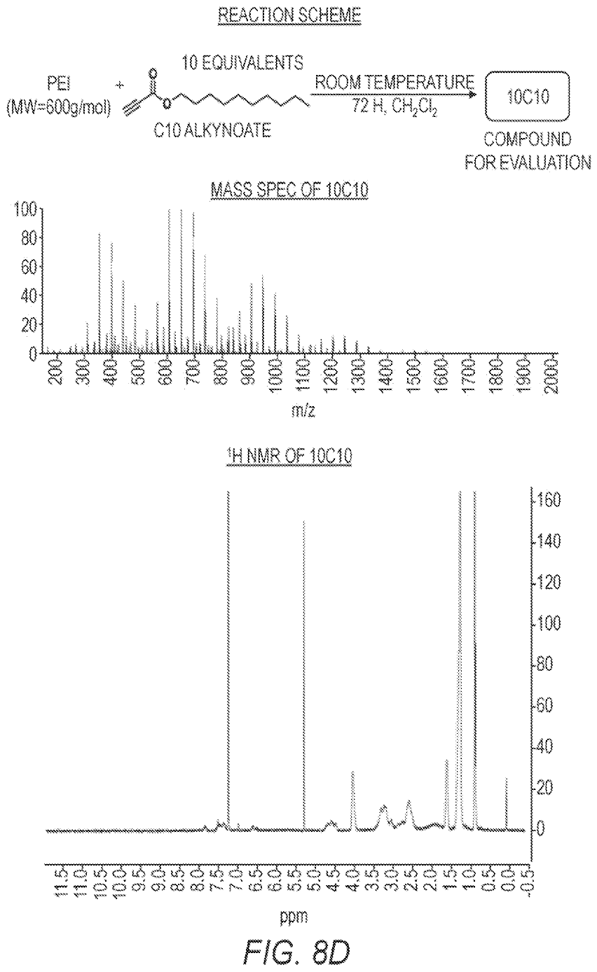

Reaction Scheme, Mass Spectrometry Data and ¹H NMR of PEI compound functionalized with 2.5C₁₀ is shown in FIG. 8A.

Synthetic Procedure for 5C₁₀. To PEI (36 mg, 0.06 mol, 1 eq) in dichloromethane (750 μL) was added a solution of C₁₀ alkynoate (63.096 mg of alkynoate in 250 μL of dichloromethane, 0.3 mmol, 5 eq). The mixture was stirred for 72 h at room temperature and was then concentrated under a high-pressure vacuum to yield 5C₁₀.

Reaction Scheme, Mass Spectrometry Data and ¹H NMR of PEI compound functionalized with 5C₁₀ is shown in FIG. 8B.

Synthetic Procedure for 7.5C₁₀. To PEI (36 mg, 0.06 mol, 1 eq) in dichloromethane (625 μL) was added a solution of C₁₀ alkynoate (94.644 mg of alkynoate in 375 μL of dichloromethane, 0.45 mmol, 7.5 eq). The mixture was stirred for 72 h at room temperature and was then concentrated under a high-pressure vacuum to yield 7.5C₁₀.

Reaction Scheme, Mass Spectrometry Data and ¹H NMR of PEI compound functionalized with 7.5C₁₀ is shown in FIG. 8C.

Synthetic Procedure for 10C₁₀. To PEI (36 mg, 0.06 mol, 1 eq) in dichloromethane (500 μL) was added a solution of C₁₀ alkynoate (126.192 mg of alkynoate in 500 μL of dichloromethane, 0.6 mmol, 10 eq). The mixture was stirred for 72 h at room temperature and was then concentrated under a high-pressure vacuum to yield 10C₁₀.

Reaction Scheme, Mass Spectrometry Data and ¹H NMR of PEI compound functionalized with 10C₁₀ is shown in FIG. 8D.

Synthetic Procedure for 2.5C₁₁. To PEI (36 mg, 0.06 mol, 1 eq) in dichloromethane (875 μL) was added a solution of C₁₁ alkynoate (33.651 mg of alkynoate in 125 μL of dichloromethane, 0.15 mmol, 2.5 eq). The mixture was stirred for 72 h at room temperature and was then concentrated under a high-pressure vacuum to yield 2.5C₁₁.

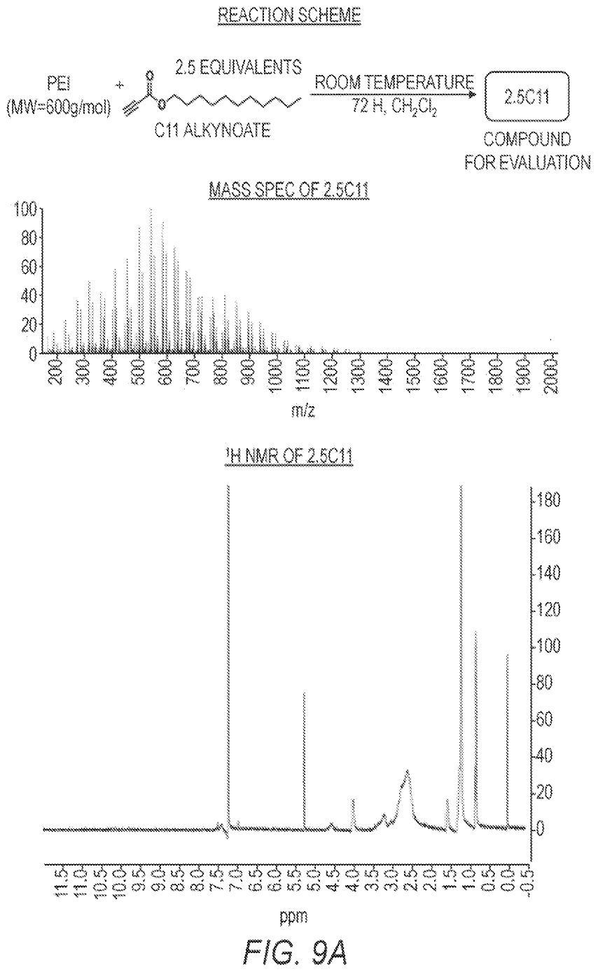

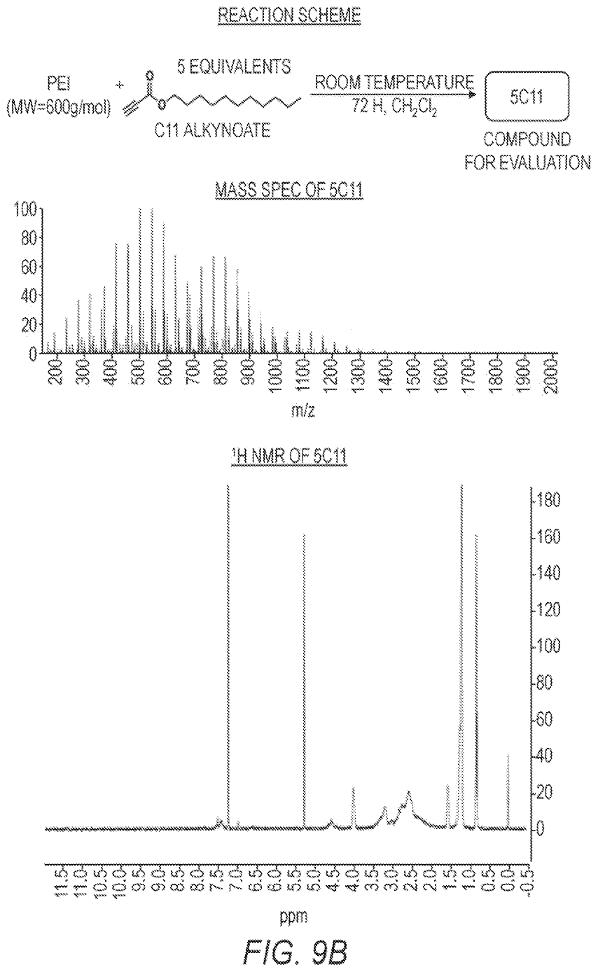

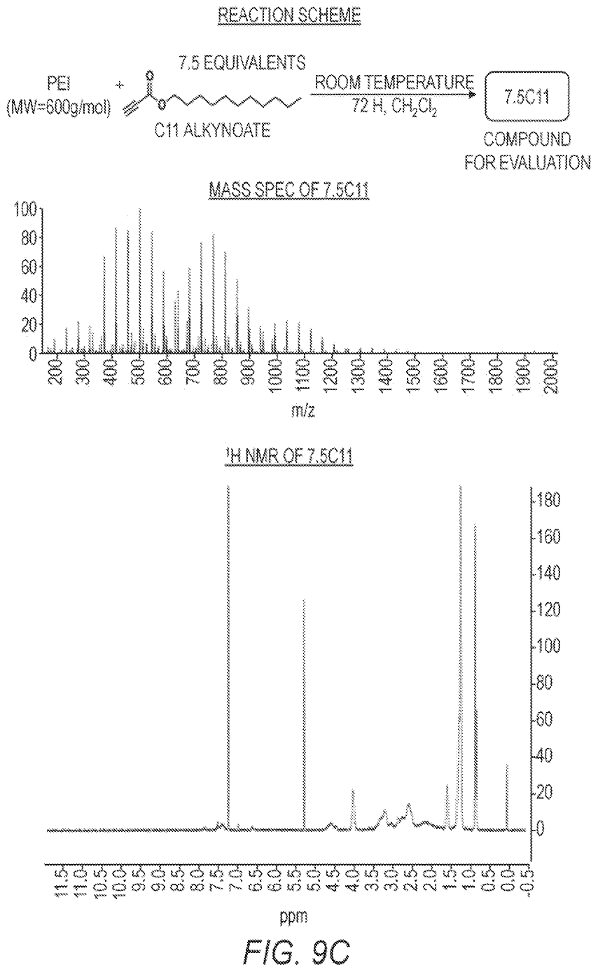

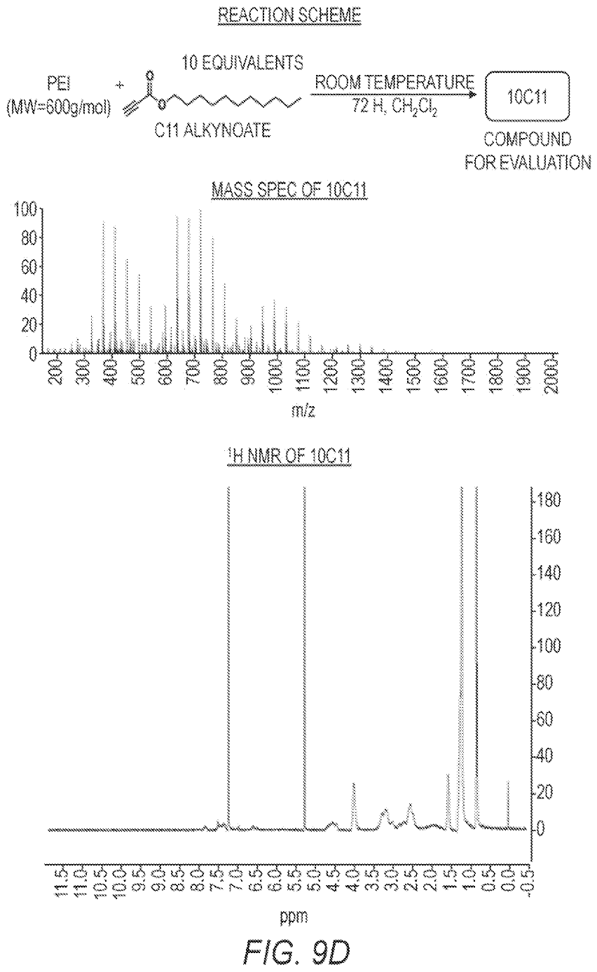

Reaction Scheme, Mass Spectrometry Data and ¹H NMR of PEI compound functionalized with 2.5C₁₁ is shown in FIG. 9A.

Synthetic Procedure for 5C₁₁. To PEI (36 mg, 0.06 mol, 1 eq) in dichloromethane (750 μL) was added a solution of C₁₁ alkynoate (67.302 mg of alkynoate in 250 μL of dichloromethane, 0.3 mmol, 5 eq). The mixture was stirred for 72 h at room temperature and was then concentrated under a high-pressure vacuum to yield 5C₁₁.

Reaction Scheme, Mass Spectrometry Data and ¹H NMR of PEI compound functionalized with 5C₁₁ is shown in FIG. 9B.

Synthetic Procedure for 7.5C₁₁. To PEI (36 mg, 0.06 mol, 1 eq) in dichloromethane (625 μL) was added a solution of C₁₁ alkynoate (100.953 mg of alkynoate in 375 μL of dichloromethane, 0.45 mmol, 7.5 eq). The mixture was stirred for 72 h at room temperature and was then concentrated under a high-pressure vacuum to yield 7.5C₁₁.

Reaction Scheme, Mass Spectrometry Data and ¹H NMR of PEI compound functionalized with 7.5C₁₁ is shown in FIG. 9C.

Synthetic Procedure for 10C₁₁. To PEI (36 mg, 0.06 mol, 1 eq) in dichloromethane (500 μL) was added a solution of C₁₁ alkynoate (134.604 mg of alkynoate in 500 μL of dichloromethane, 0.6 mmol, 10 eq). The mixture was stirred for 72 h at room temperature and was then concentrated under a high-pressure vacuum to yield 10C₁₁.

Reaction Scheme, Mass Spectrometry Data and ¹H NMR of PEI compound functionalized with 10C₁₁ is shown in FIG. 9D.

Synthetic Procedure for 2.5C₁₂. To PEI (36 mg, 0.06 mol, 1 eq) in dichloromethane (875 μL) was added a solution of C₁₂ alkynoate (35.756 mg of alkynoate in 125 μL of dichloromethane, 0.15 mmol, 2.5 eq). The mixture was stirred for 72 h at room temperature and was then concentrated under a high-pressure vacuum to yield 2.5C₁₂.

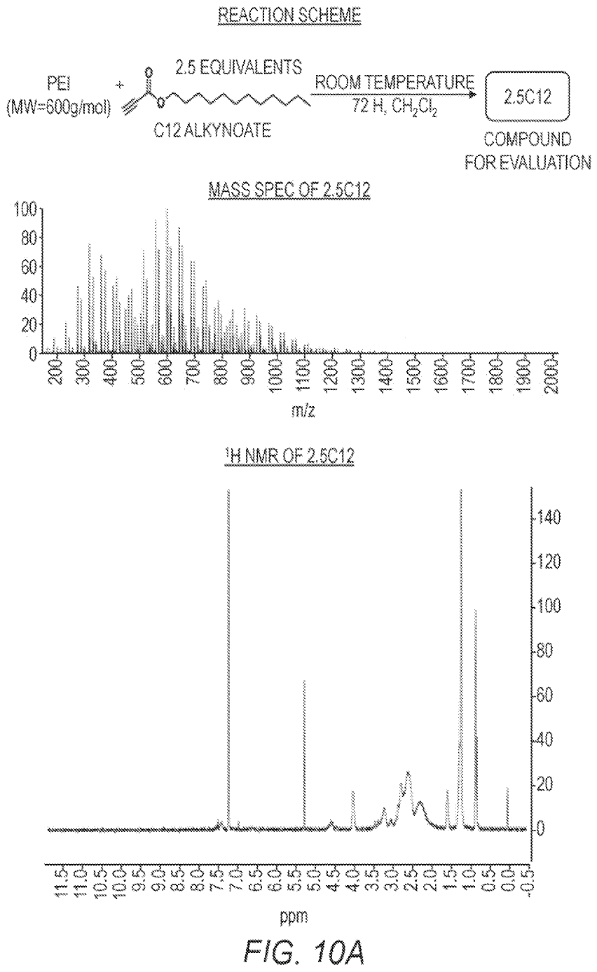

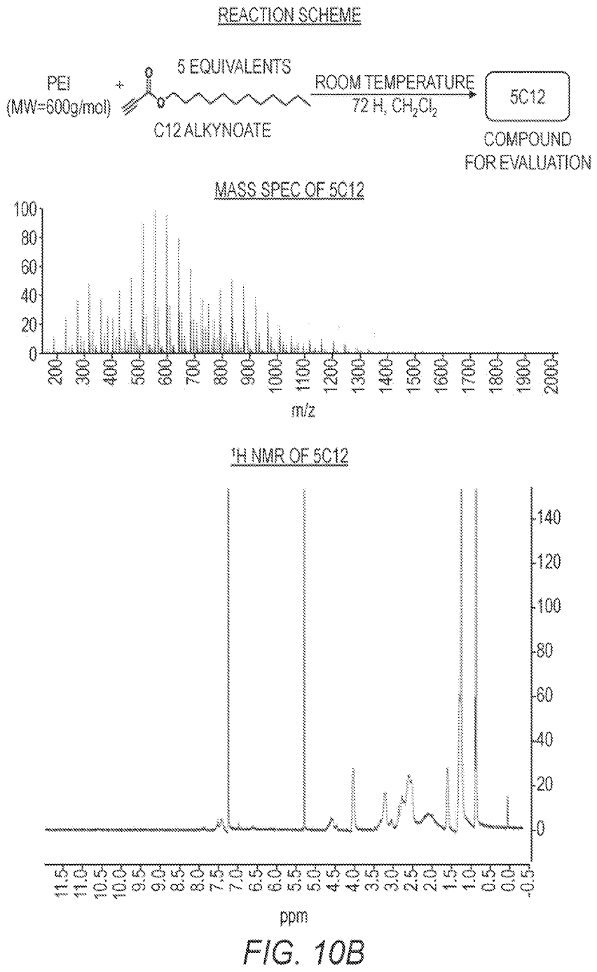

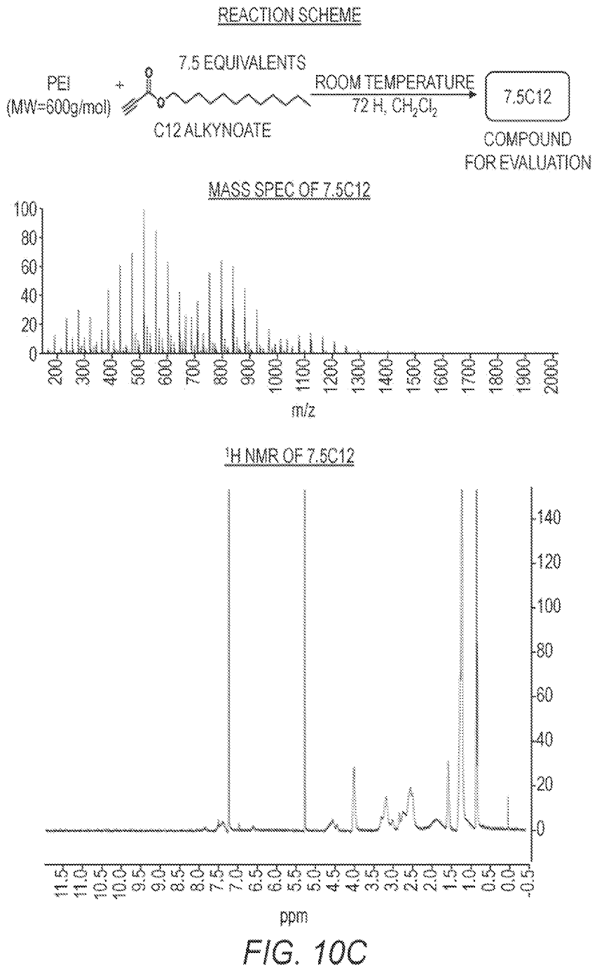

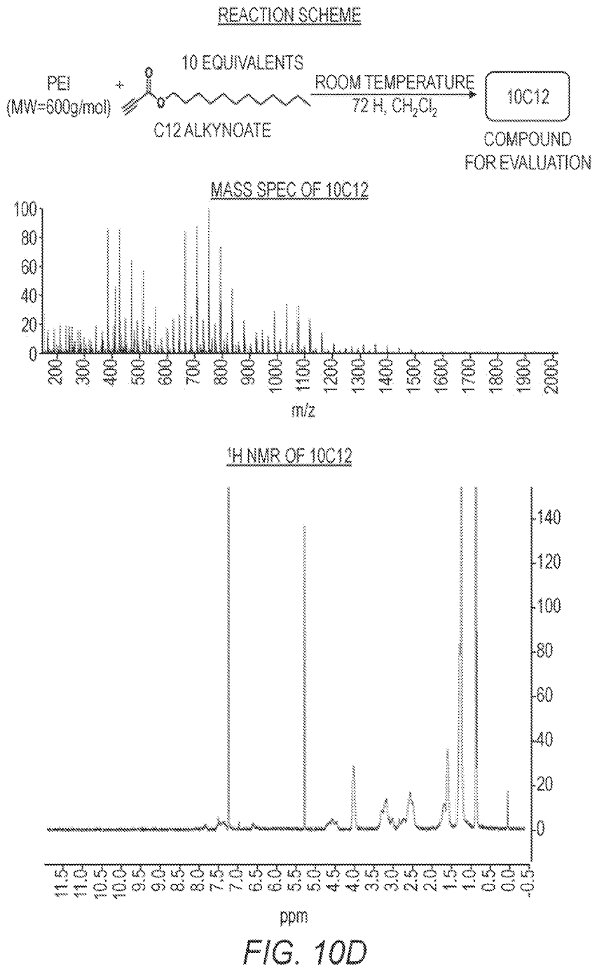

Reaction Scheme, Mass Spectrometry Data and ¹H NMR of PEI compound functionalized with 2.5C₁₂ is shown in FIG. 10A.

Synthetic Procedure for 5C₁₂. To PEI (36 mg, 0.06 mol, 1 eq) in dichloromethane (750 μL) was added a solution of C₁₂ alkynoate (71.511 mg of alkynoate in 250 μL of dichloromethane, 0.3 mmol, 5 eq). The mixture was stirred for 72 h at room temperature and was then concentrated under a high-pressure vacuum to yield 5C₁₂.

Reaction Scheme, Mass Spectrometry Data and ¹H NMR of PEI compound functionalized with 5C₁₂ is shown in FIG. 10B.

Synthetic Procedure for 7.5C₁₂. To PEI (36 mg, 0.06 mol, 1 eq) in dichloromethane (625 μL) was added a solution of C₁₂ alkynoate (107.267 mg of alkynoate in 375 μL of dichloromethane, 0.45 mmol, 7.5 eq). The mixture was stirred for 72 h at room temperature and was then concentrated under a high-pressure vacuum to yield 7.5C₁₂.

Reaction Scheme, Mass Spectrometry Data and ¹H NMR of PEI compound functionalized with 7.5C₁₂ is shown in FIG. 10C.

Synthetic Procedure for 10C₁₂. To PEI (36 mg, 0.06 mol, 1 eq) in dichloromethane (500 μL) was added a solution of C₁₂ alkynoate (143.022 mg of alkynoate in 500 μL of dichloromethane, 0.6 mmol, 10 eq). The mixture was stirred for 72 h at room temperature and was then concentrated under a high-pressure vacuum to yield 10C₁₂.

Reaction Scheme, Mass Spectrometry Data and ¹H NMR of PEI compound functionalized with 10C₁₂ is shown in FIG. 10D.

Synthetic Procedure for 2.5C₁₃. To PEI (36 mg, 0.06 mol, 1 eq) in dichloromethane (875 μL) was added a solution of C₁₃ alkynoate (37.860 mg of alkynoate in 125 μL of dichloromethane, 0.15 mmol, 2.5 eq). The mixture was stirred for 72 h at room temperature and was then concentrated under a high-pressure vacuum to yield 2.5C₁₃.

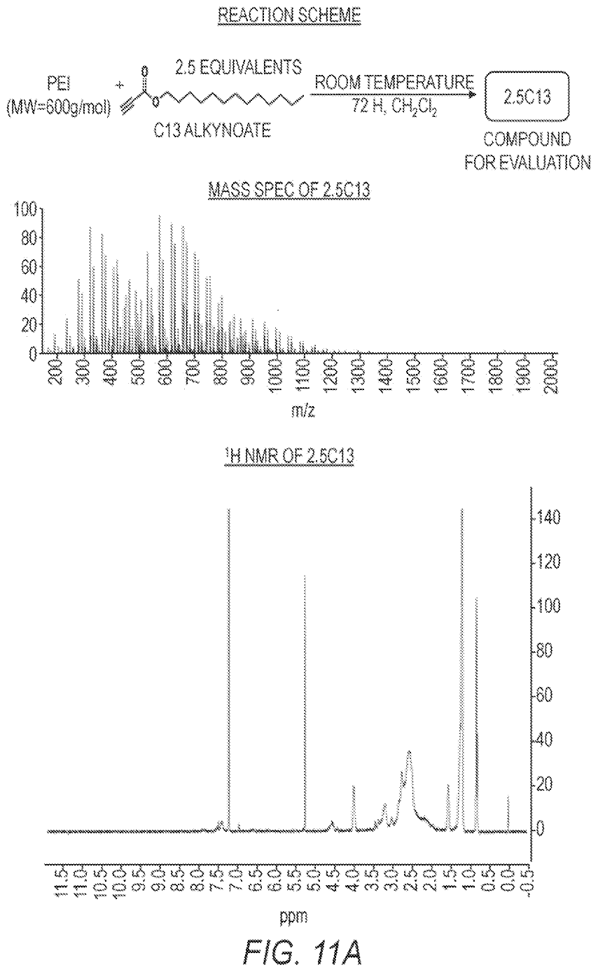

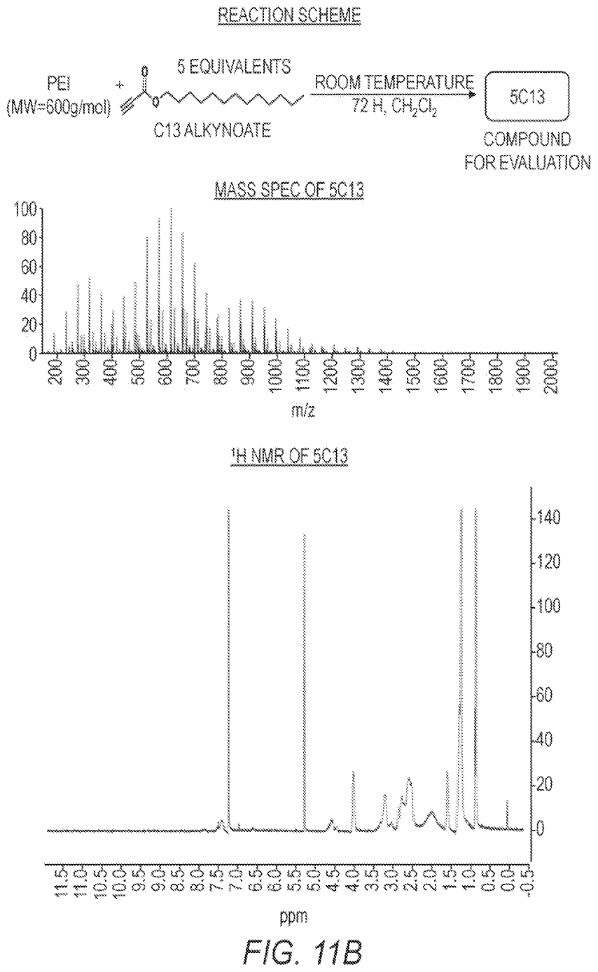

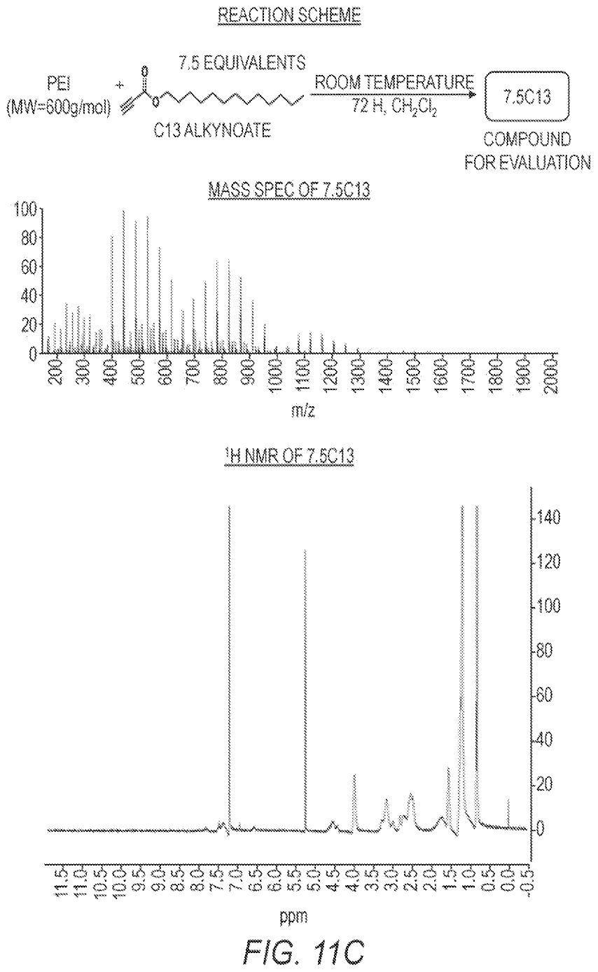

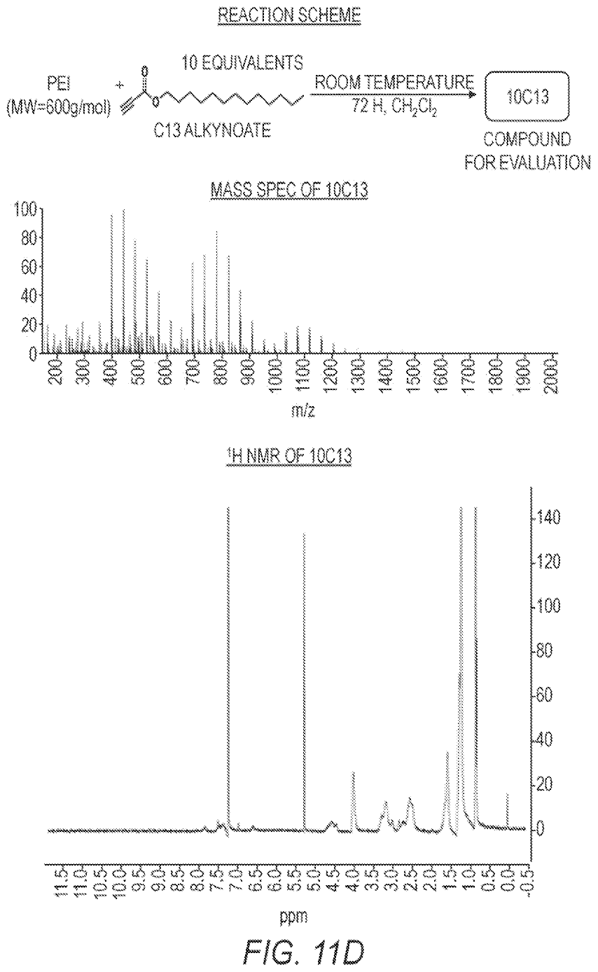

Reaction Scheme, Mass Spectrometry Data and ¹H NMR of PEI compound functionalized with 2.5C₁₃ is shown in FIG. 11A.

Synthetic Procedure for 5C₁₃. To PEI (36 mg, 0.06 mol, 1 eq) in dichloromethane (750 μL) was added a solution of C₁₃ alkynoate (75.720 mg of alkynoate in 250 μL of dichloromethane, 0.3 mmol, 5 eq). The mixture was stirred for 72 h at room temperature and was then concentrated under a high-pressure vacuum to yield 5C₁₃.

Reaction Scheme, Mass Spectrometry Data and ¹H NMR of PEI compound functionalized with 5C₁₃ is shown in FIG. 11B.

Synthetic Procedure for 7.5C₁₃. To PEI (36 mg, 0.06 mol, 1 eq) in dichloromethane (625 μL) was added a solution of C₁₃ alkynoate (113.580 mg of alkynoate in 375 μL of dichloromethane, 0.45 mmol, 7.5 eq). The mixture was stirred for 72 h at room temperature and was then concentrated under a high-pressure vacuum to yield 7.5C₁₃.

Reaction Scheme, Mass Spectrometry Data and ¹H NMR of PEI compound functionalized with 7.5C₁₃ is shown in FIG. 11C.

Synthetic Procedure for 10C₁₃. To PEI (36 mg, 0.06 mol, 1 eq) in dichloromethane (500 μL) was added a solution of C₁₃ alkynoate (151.44 mg of alkynoate in 500 μL of dichloromethane, 0.6 mmol, 10 eq). The mixture was stirred for 72 h at room temperature and was then concentrated under a high-pressure vacuum to yield 10C₁₃.

Reaction Scheme, Mass Spectrometry Data and ¹H NMR of PEI compound functionalized with 10C₁₃ is shown in FIG. 11D.

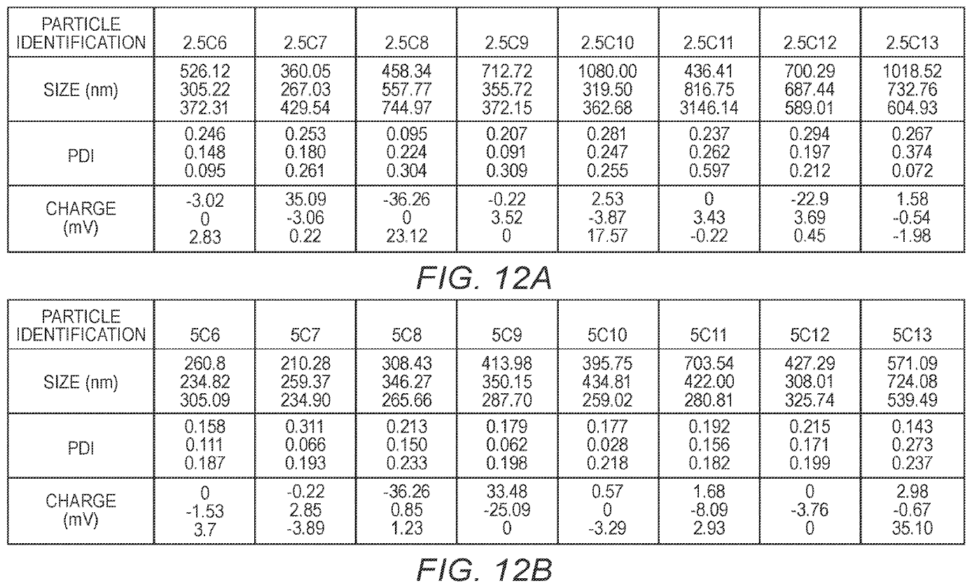

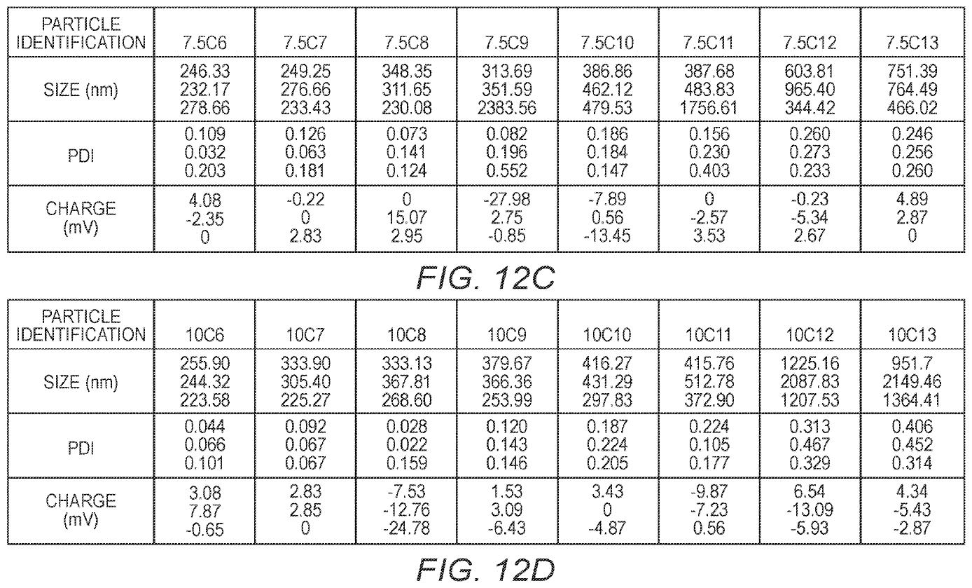

Particle characterization data for each of 2.5 equivalents, 5 equivalents 7 equivalents and 10 equivalents of the compound library is shown in FIGS. 12A-12D. DLS data was collected for particles loaded with mRNA using the U251 cell line and measured 3 different times.

### Example 3. In Vitro Transfection

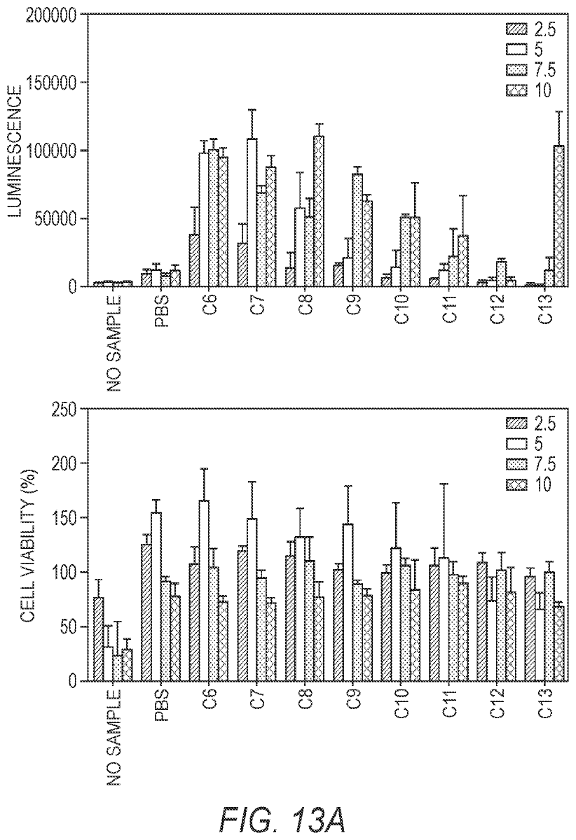

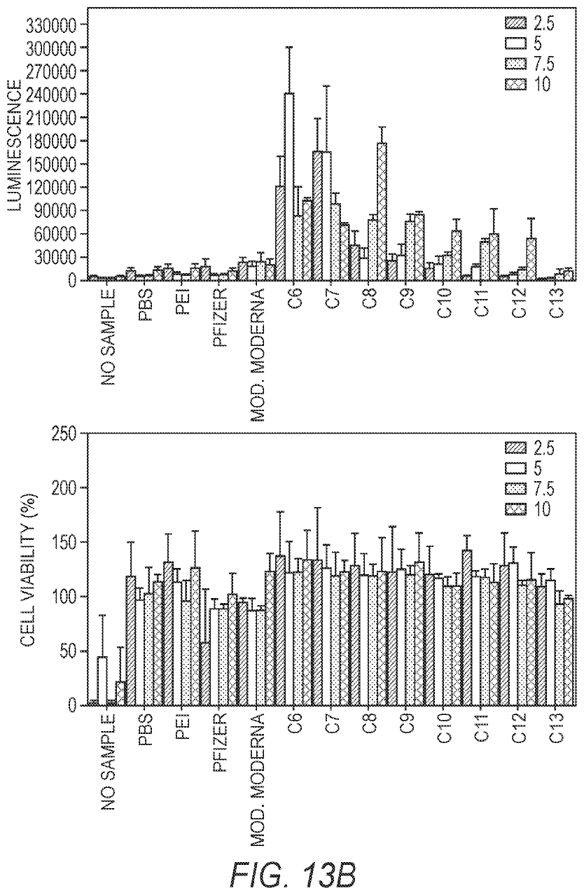

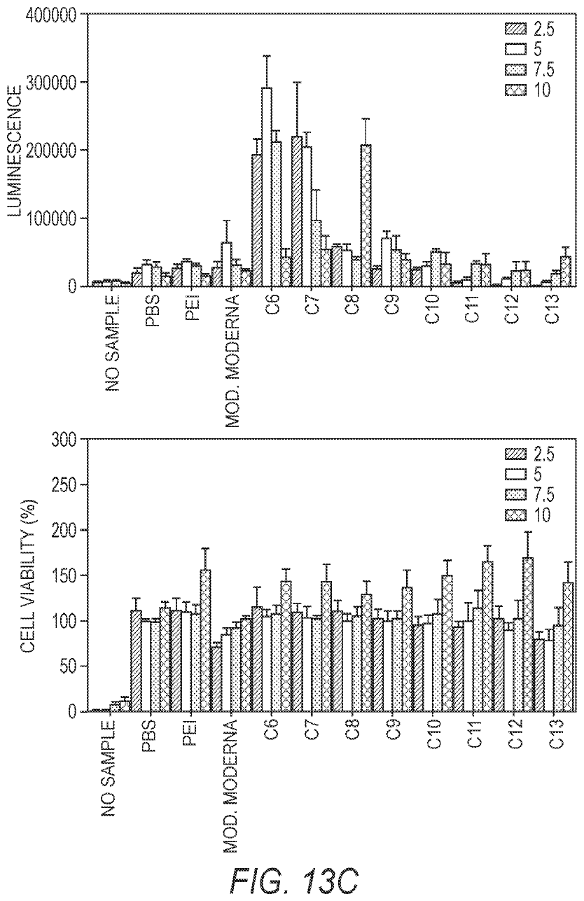

Transfection data for example functionalized PEI particles was measured in three trials, shown in FIGS. 13A-13C. 50 nanograms of mRNA were loaded in each well, with variable tail to PEI ratio of 2.5, 5, 7.5 and 10 equivalents in U251 cells. Translation and cell viability were measured, with measurements compared to Pfizer and Modified Moderna particles (FIG. 13B) and Moderna particles (FIG. 13C). Cell viability of functionalized PEI particles show strong cell viability, with many formulations outperforming translation by unmodified PEI, Moderna particles, Pfizer particles, and modified Moderna particles, formulations for Moderna and Pfizer particles are available at Schoenmaker et al., Int'l J. of Pharma, 601:120586 (2021)(Table 1), incorporated herein by reference. Modified Moderna particles comprised SM-102 (50 mol %); DOPE (10 mol %); cholesterol (38.5 mol %); DMG-PEG-2000 (1.5 mol %). Moderna particles comprised: SM-102 (50 mol %); DSPC (10 mol %); cholesterol (38.5 mol %); DMG-PEG-2000 (1.5 mol %). Pfizer particles comprised: ALC-0315 (46.3 mol %), ALC-0159=(9.4 mol %), cholesterol (42.7 mol %); 2-[(polyethylene glycol)-2000]-N,N ditetradecylacetamide (1.6 mol %).

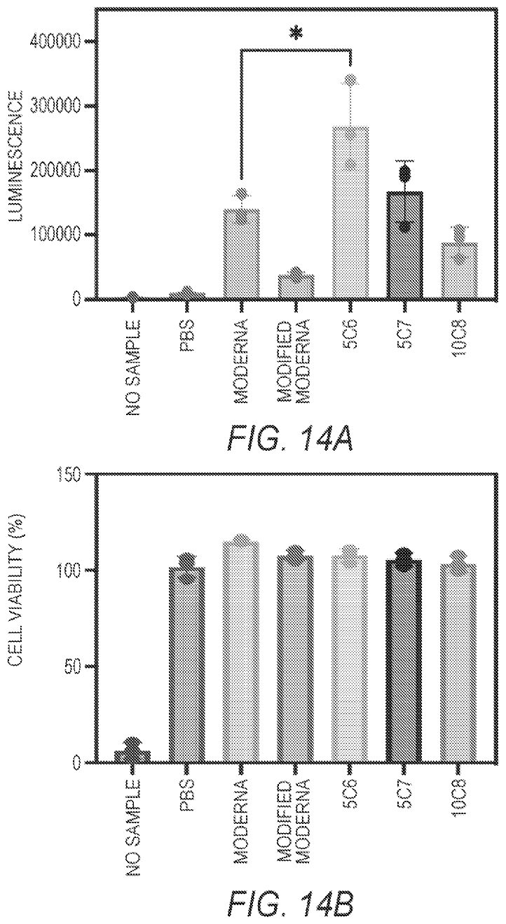

Example particles functionalized with 5C₆, 5C₇, and 10C₈ loaded with 50 ng mRNA were measured for translation and cell viability, with performance compared to Moderna formulation in translation (FIG. 14A) and cell viability (FIG. 14B). As shown in FIG. 14A, the particles functionalized with 5 equivalents of C₆ alkynoate outperformed Moderna particles and modified Moderna particles.

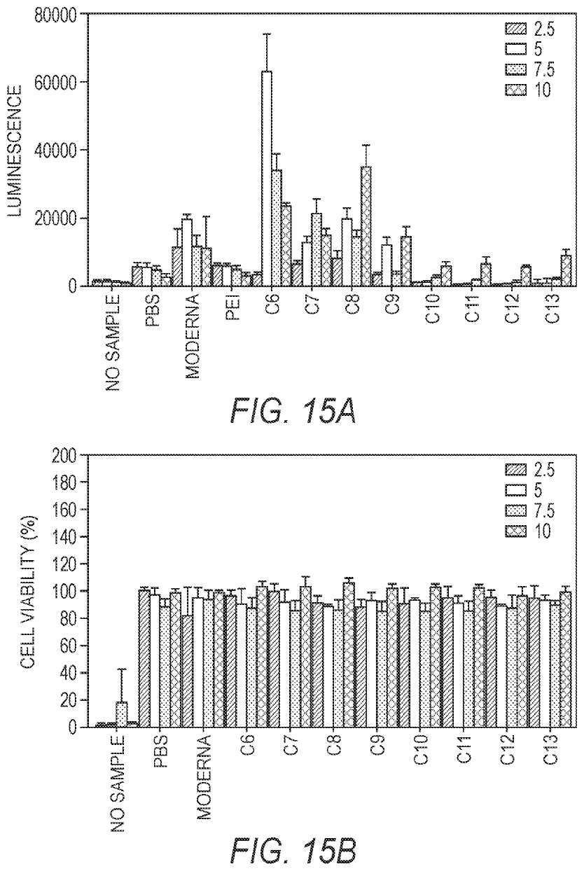

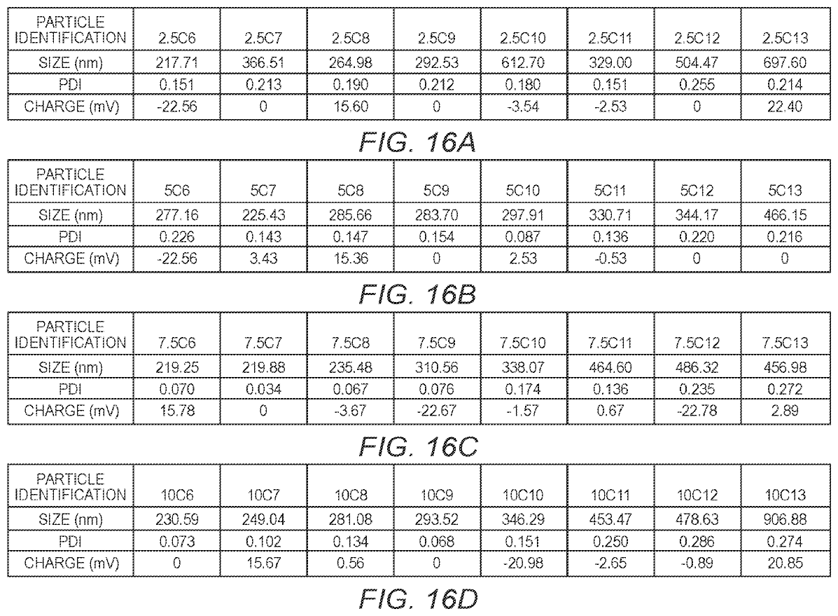

Transfection was further explored in Madin-Darby canine kidney (MDCK) cells (FIGS. 15A-15B). Collective transfection data for a library of functionalized PEI particles using C₆-C₁₃ alkynoates with variable tail to PEI ratio and 50 ng of mRNA per well were measured, with translation measurements for several functionalized PEI particles outperforming Moderna particles, and strong cell viability. DLS characterized the C₆-C₁₃ alkynoate modified particles in the MDCK cell line for particles loaded with mRNA, including size (nm), polydispersity index (PDI) and charge measured in mV. (FIGS. 16A-16D).

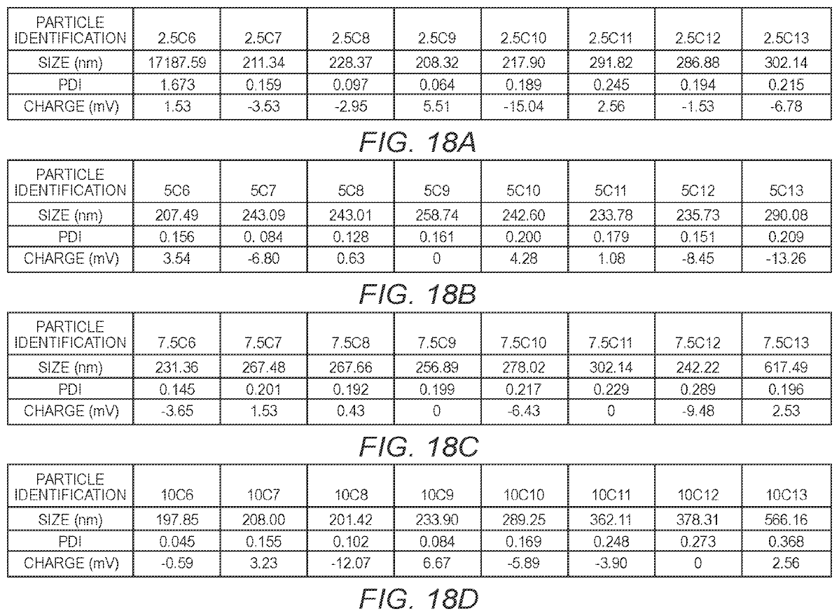

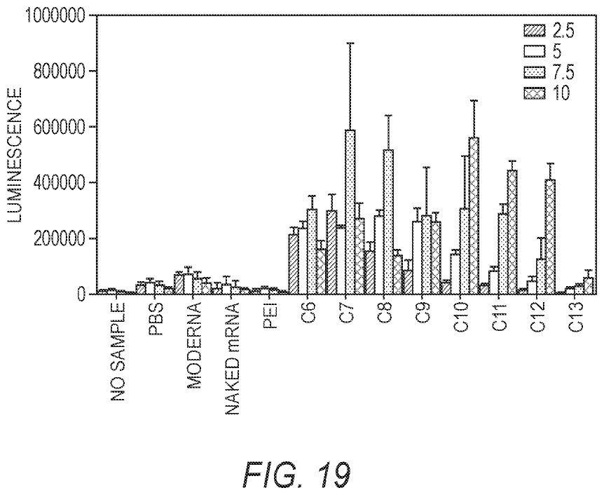

DLS particle characterization data of the functionalized PEI particles prepared with PEG and mRNA cargo are shown in FIGS. 18A-18D. The figures show collective transfection data for library of functionalized PEI particles with 0.25% PEG using C₆-C₁₃ alkynoates with variable tail to PEI ratio and 50 ng mRNA per well in U251 cells, with translation measured.

### Example 4. In Vivo Studies of Example Functionalized PEI Particles

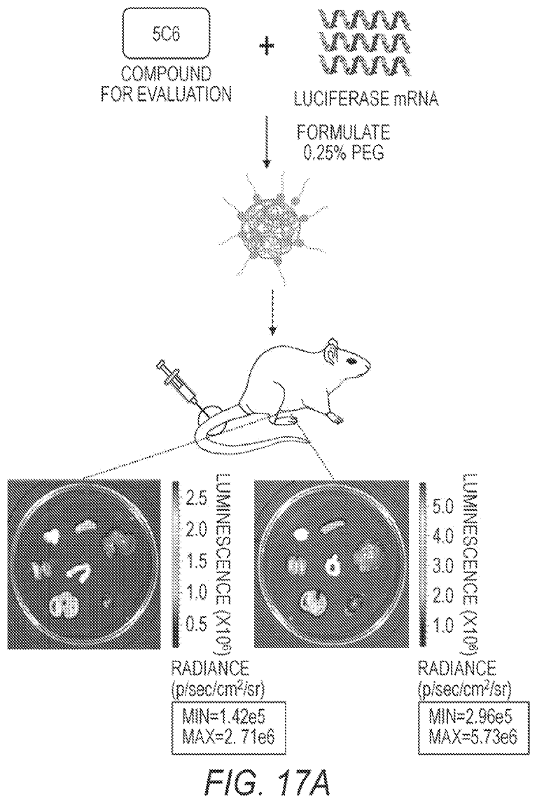

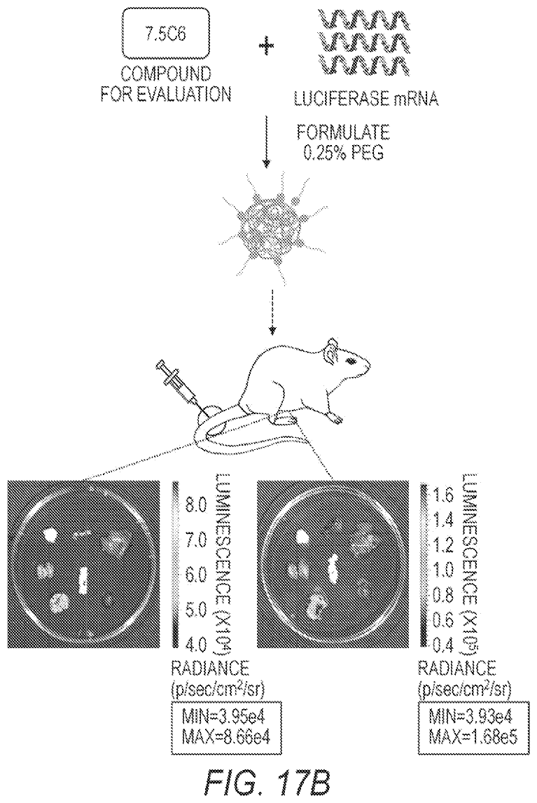

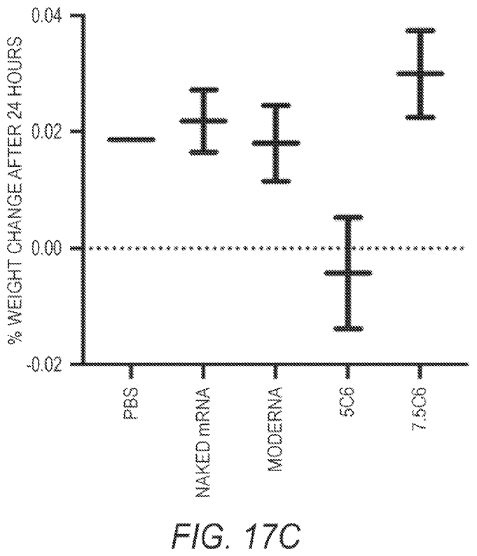

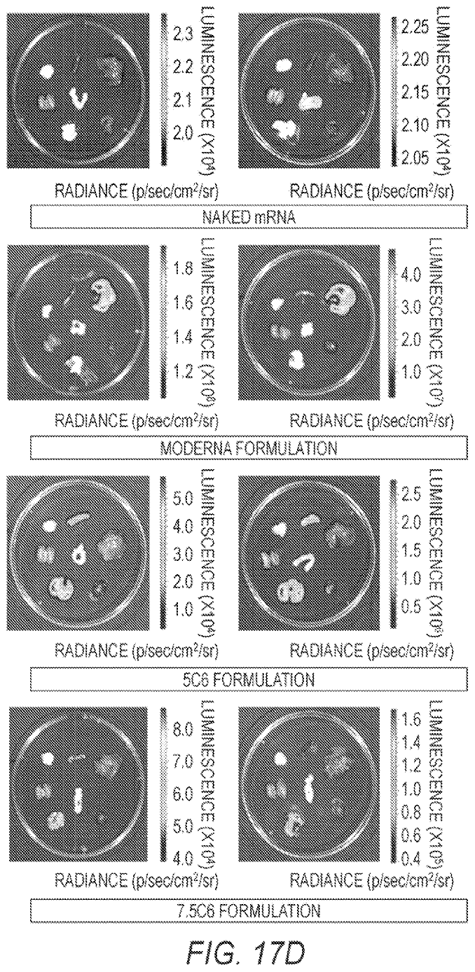

Trial 1. In vivo intravenous screening of 5 equivalents of C₆ alkynoate modified PEI particles formulated with 0.25% PEG and luciferase mRNA is illustrated in FIG. 17A. Two mice were injected with functionalized PEI particles loaded with 0.5 mg/kg luciferase mRNA with a particle size of 152 nm and zeta potential of −9.6 mV. PEI functionalized with 7.5 equivalents of C₆ alkynoate were likewise studied in two mice using 0.5 mg/kg dose injected intravenously (FIG. 17B). Particles were 165.05 nm with a zeta of −5.1 mV and showed selectivity for the lung and spleen tissues of mice (FIG. 17B). The lack of change in mouse weight measured before and 24 hours post-injection of particles shows the particles are not toxic (FIG. 17C). Measured luminescence for mouse organs shows liver expression for Moderna injection, with lung and spleen selectivity for example formulations of the present invention, and no tissue expression for mRNA delivered alone (FIG. 17D).

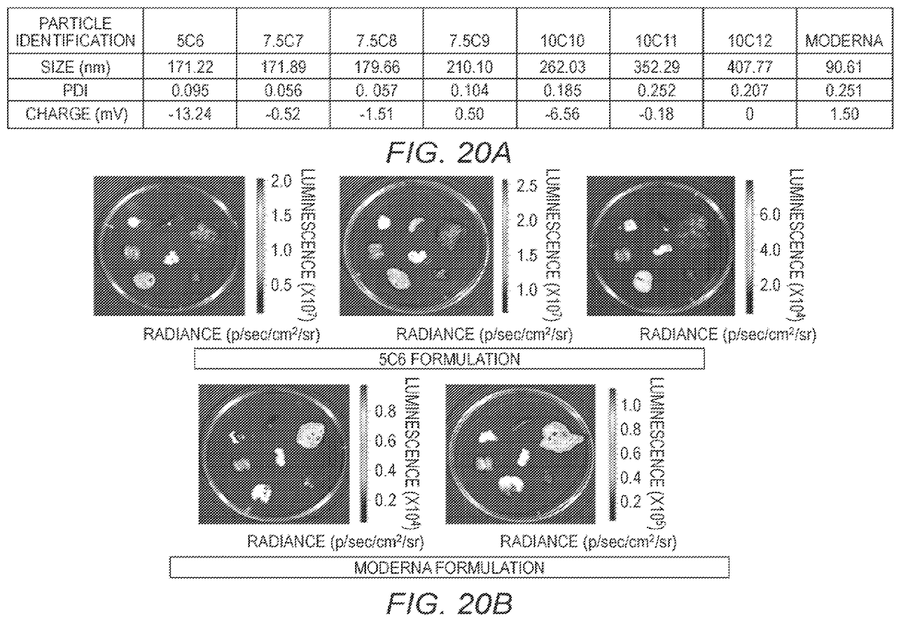

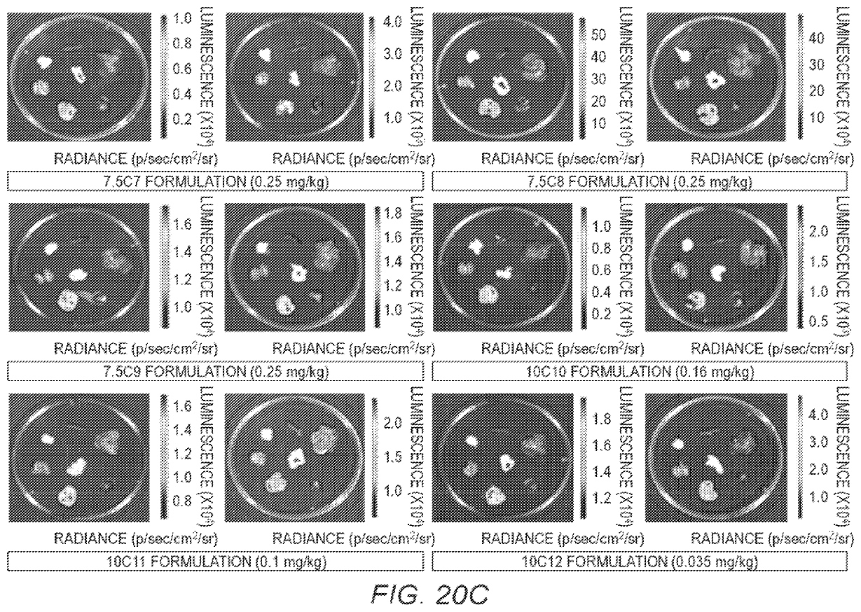

Trial 2. A second trial of in vivo intravenous injection of two mice with alkynoate modified PEI particles formulated with 0.25% PEG and luciferase mRNA was performed. DLS data for example particles loaded with mRNA after dialysis is shown in FIG. 20A. Images of organs harvested from mice injected with PEI modified with 5 equivalents of C₆ alkynoate shows activity in lung and spleen tissues of mice (FIG. 20B). Additional exemplary functionalized PEI formulations of 7.5C₇, 7.5C₈, 7.5C₉, 10C₁₀, 10C₁₁, 10C₁₂ were injected, with certain formulations showing full selectivity for lung tissue. (FIG. 20C). Measurements of change in mice weight before injection and 24 hours post-injection of particles at a 0.5 mg/kg dose indicated a lack toxicity relative to PBS and Moderna particles.

### Example 5. A Lung-Expressing mRNA Delivery Platform with Tunable Activity in Hypoxic Environments

Messenger RNA (mRNA) delivery platforms often facilitate protein expression in the liver following intravenous injection and have been optimized for use in normally oxygenated cells. However, there is a growing need for mRNA therapy in diseases affecting non-liver organs, such as the lungs. Additionally, many diseases are characterized by hypoxia, a state of abnormally low oxygenation in cells and tissues that can reduce the efficacy of mRNA therapies by upwards of 80%. Hypoxia—a state of low oxygenation that is present across multiple types of diseases such as cystic fibrosis, asthma, and solid tumors¹⁷⁻¹⁹—can decrease the efficacy of mRNA nanoparticle therapies by upwards of 80%.^{20,21} The present example reports a lung-expressing mRNA nanoparticle delivery system (5C6) whose properties can be readily tuned for optimal expression in hypoxic environments. Briefly, the study begins with the synthesis and characterization of a novel aminoacrylate polymer, 5C6, that can be effectively complexed into mRNA polymer nanoparticles. The efficacy and mechanism of 5C6 was studied, including analysis of the cellular association, endocytosis, endosomal escape, and translation in a lung cell line. 5C6 was then evaluated under hypoxic conditions and address hypoxia-related deficits in efficacy by making this system tunable with adenosine triphosphate (ATP). Finally, the study was concluded with an in vivo analysis of mRNA expression, biodistribution, and tolerability of the 5C6 platform. Together, a novel mRNA delivery system that facilitates protein expression in the lungs is presented and is tunable for hypoxic environments. In undertaking this project, the goal was not only to establish and characterize a tunable lung-expressing mRNA delivery system but also to more broadly highlight the importance of considering and overcoming hypoxia in the development of next-generation mRNA delivery systems.

### Results and Discussion

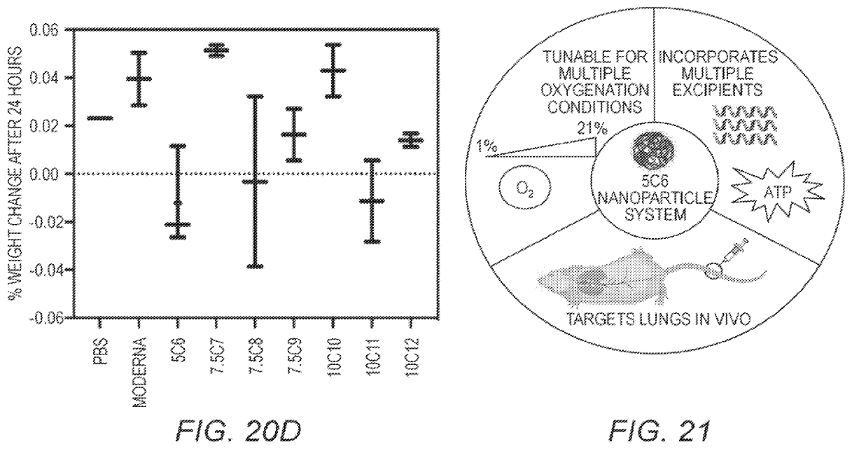

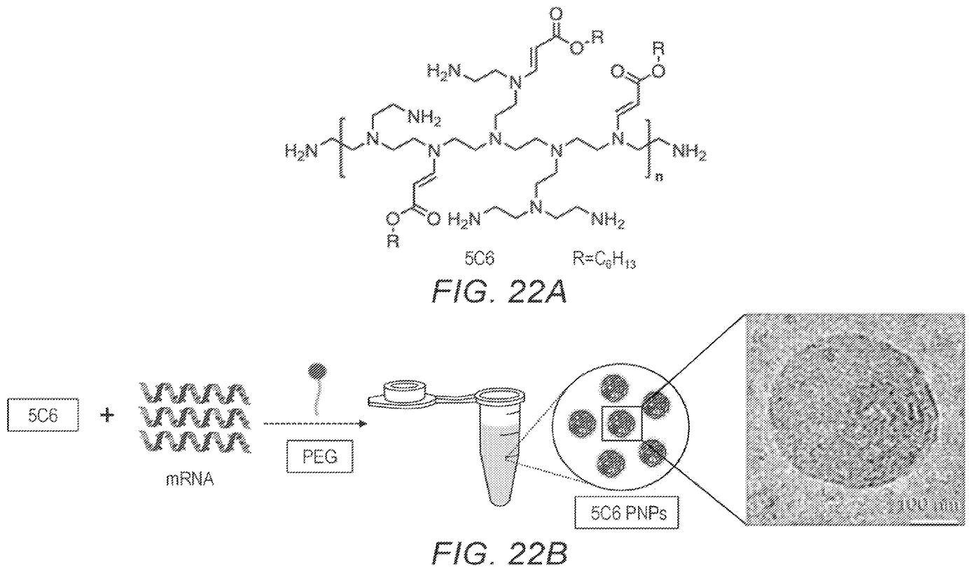

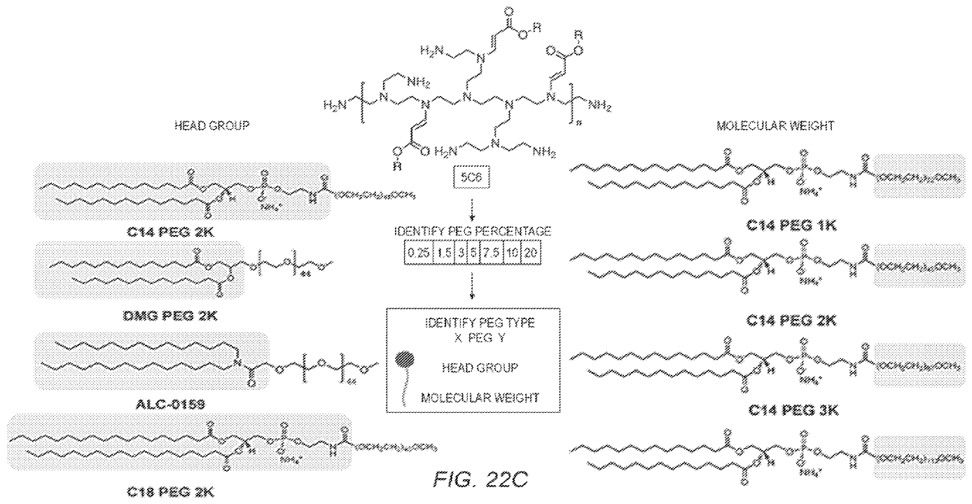

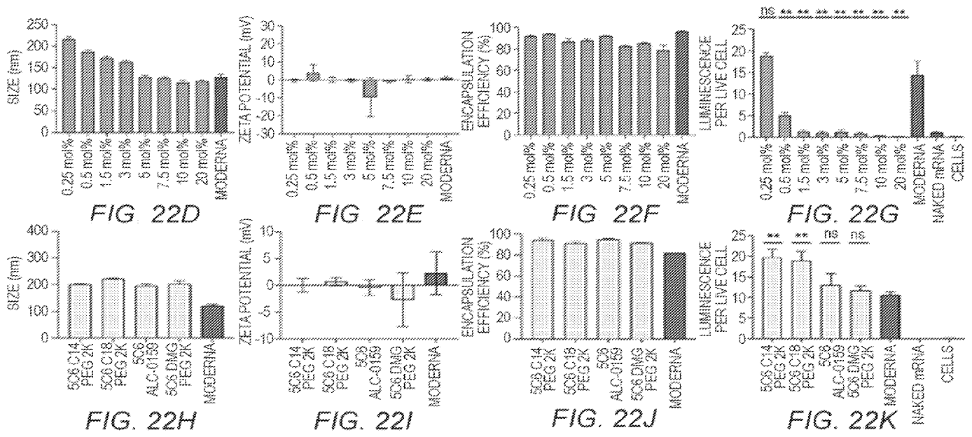

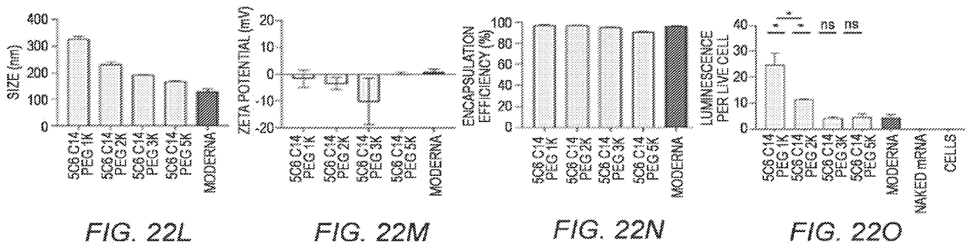

Toward developing a lung-expressing and hypoxia-tunable mRNA polymeric nanoparticle (PNP), a novel polymer named 5C6 was designed and synthesized by reacting low molecular weight branched polyethyleneimine (PEI) with a 6-carbon alkynoate tail under mild reaction conditions (FIGS. 22A, 28A-28B, 29). In brief, 5C6 incorporates several structural design properties that may be beneficial for mRNA delivery including: (1) high amine density to promote efficient complexation with mRNA,^{28,32-34} (2) ester linkages to promote tolerability,^{14,35-43} (3) hydrophobic tails to promote self-assembly,⁴⁴⁻⁴⁸ (4) polyethyleneimine (PEI)-based chemistry to facilitate delivery to the lungs,^{29,49-55} and (5) tunability so that its activity could be optimized for normoxic and hypoxic conditions.

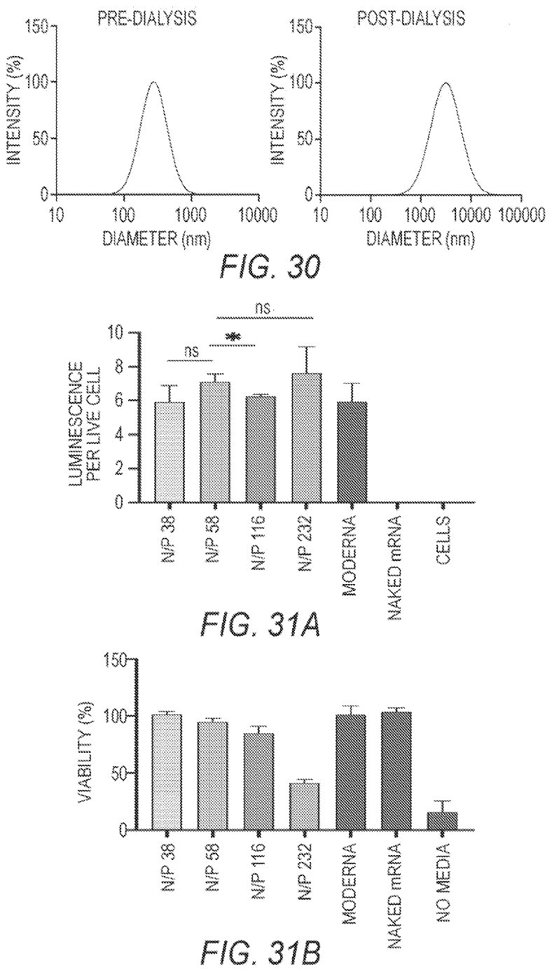

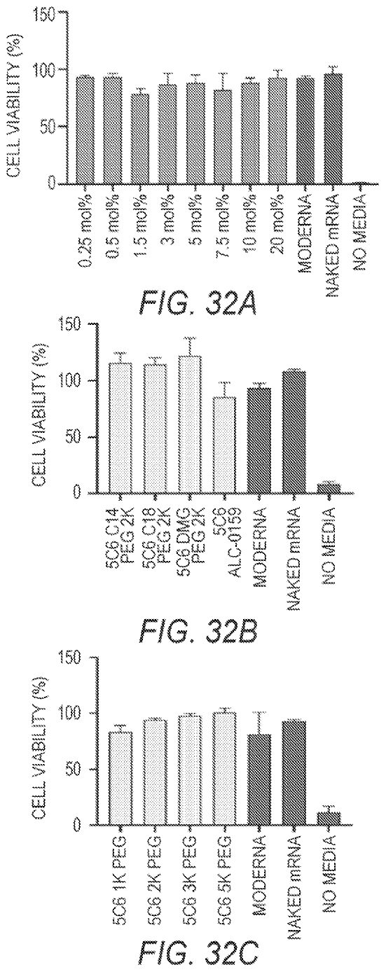

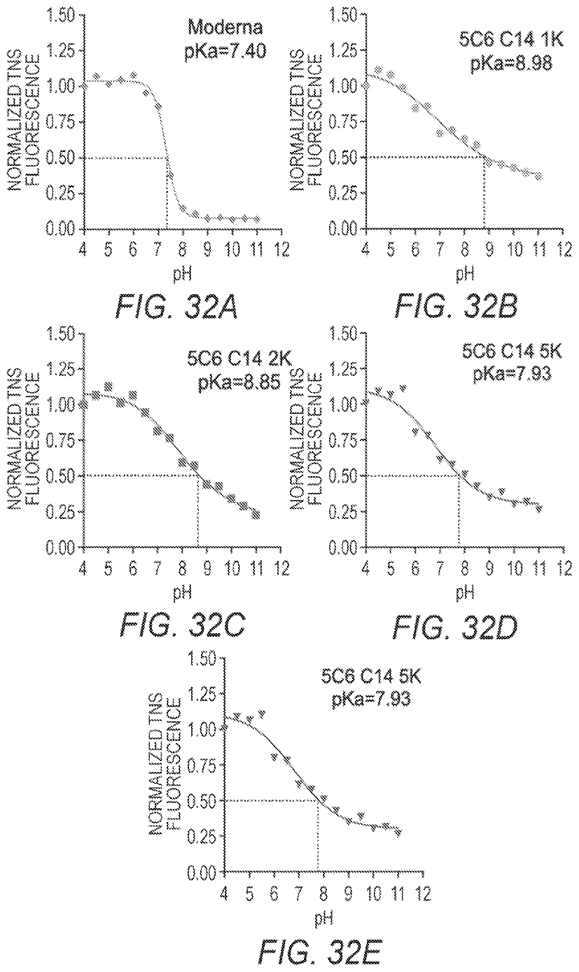

Next, the 5C6 polymer was evaluated as a nanoparticle carrier for mRNA. Particles were formulated containing 5C6 and firefly luciferase (FLuc) reporter gene mRNA. This formulation lacked stability in phosphate-buffered saline (PBS) (FIG. 30). To address this issue, it was hypothesized that the addition of polyethylene-glycol (PEG) derivatives into the formulation could afford better stability while also providing a tunable handle over PNP properties. Toward this end, a series of 5C6 PNPs was formulated and characterized with varied PEG molar ratios, head group identities, PEG molecular weights, and N/P ratios (FIGS. 22C, 31). Transmission electron microscopy was also performed to assess the shape of 5C6 PNPs (FIG. 22B). In collectively analyzing these data, several trends emerged. First, increasing the molar percentage of PEG or length of the PEG tail resulted in a general decrease in PNP size (FIGS. 22D, 2L) while head group identity did not affect size (FIG. 22H). Second, minimal changes were observed in charge across the variations in PEG molar ratio, head group, and molecular weight (FIGS. 22E, 22I, 22M). Third, mRNA encapsulation was greater than 80% for all formulations studied (FIGS. 22F, 22J, 22N). Fourth, the efficacies of the PNP formulations in delivering FLuc mRNA to A549 cells (measured by luminescence) were influenced by the PEG molar ratio, head group, and molecular weight (FIGS. 22G, 22K, 22O). All formulations were generally well tolerated (FIG. 32) with similar pKas (FIG. 33). As controls and benchmarks, each formulation was compared to phosphate-buffered saline (PBS), naked FLuc mRNA, and FLuc mRNA encapsulated within a Moderna LNP formulation analog. Based upon these results, a PEG molar ratio of 0.25%, a C14 PEG head group, and PEG molecular weights 1K, 2K, 3K, and 5K were elected for subsequent experiments as these formulations yielded the highest mRNA expression. These PNP formulations were also tested for stability using dynamic light scattering and were found to be stable for at least 72 hrs at 4° C. in diverse pH conditions (pH=5.0 and 7.4) or Dulbecco's modified Eagle medium (DMEM) supplemented with 10% fetal bovine serum (FBS) (FIG. 34).

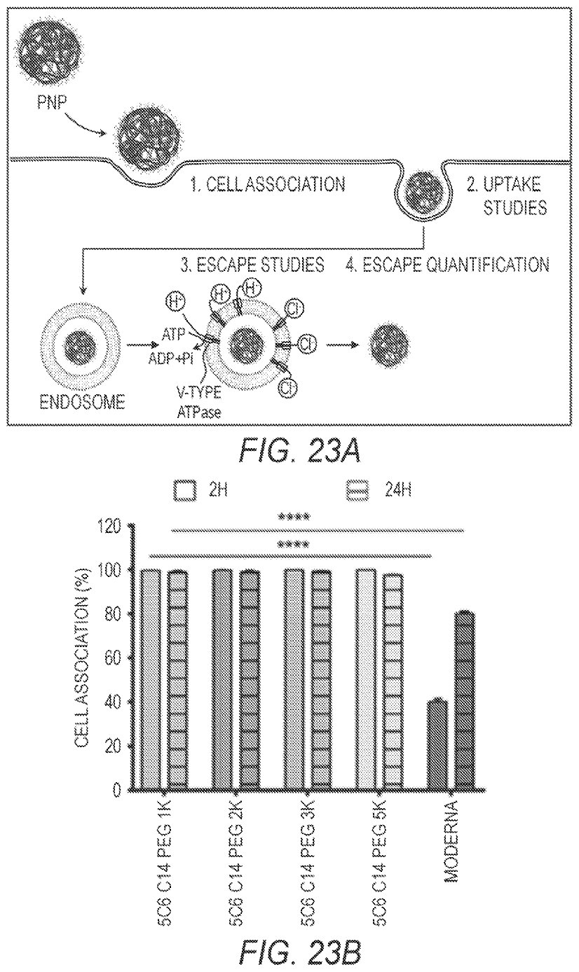

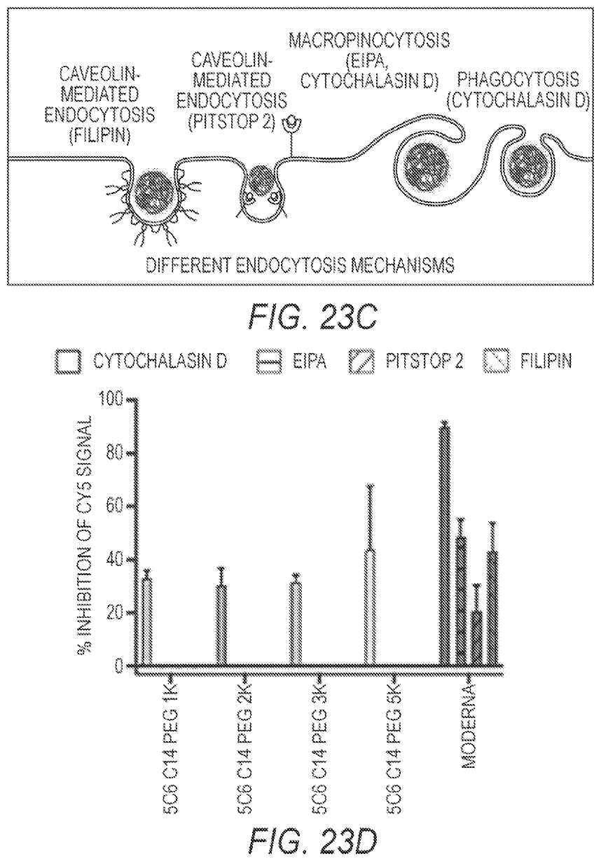

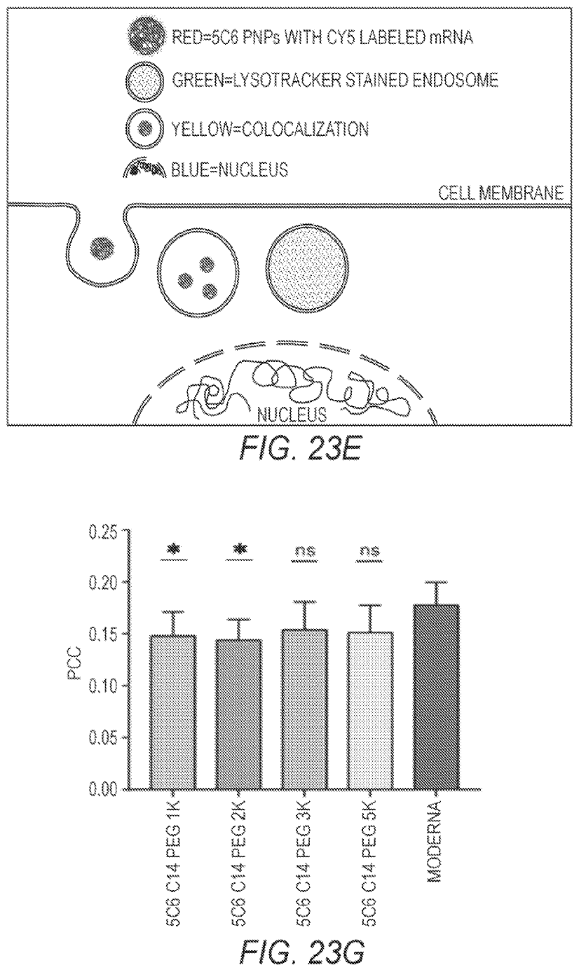

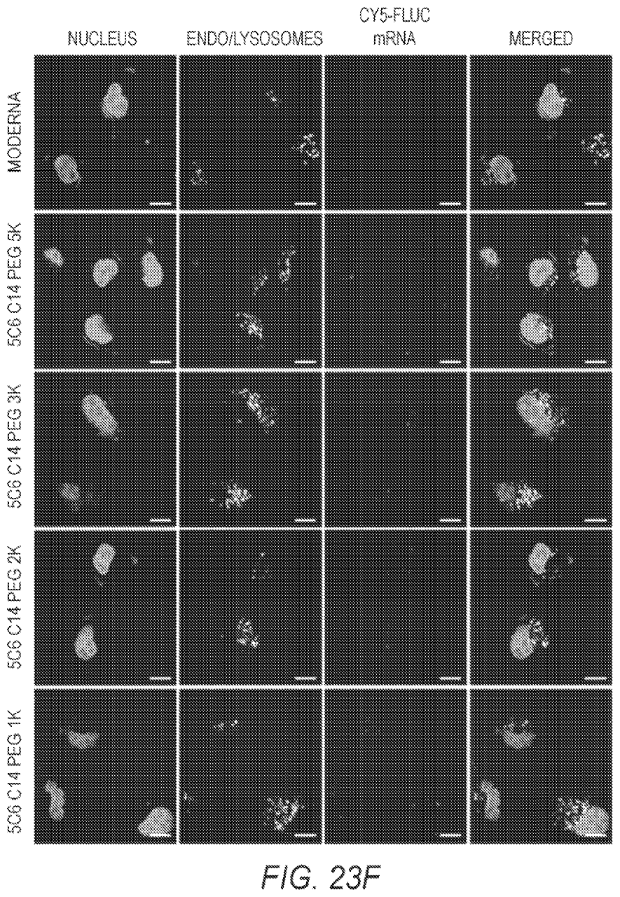

Building on these efficacy results, the mechanism by which 5C6 PNPs deliver mRNA into cells was then investigated. For mRNA encapsulated in a nanoparticle to be expressed, it must undergo several key mechanistic steps including cellular association (where nanoparticles interact with cell membranes); endocytosis (where nanoparticles are taken up into cells through various pathways); and endosomal escape (where nanoparticles are released from endosomes/lysosomes into the cytoplasm) (FIG. 23A). To better understand how the 5C6 PNPs are delivering mRNA into cells, the unique delivery properties and mechanism of 5C6 PNPs were quantified and compared to the Moderna formulation analog.

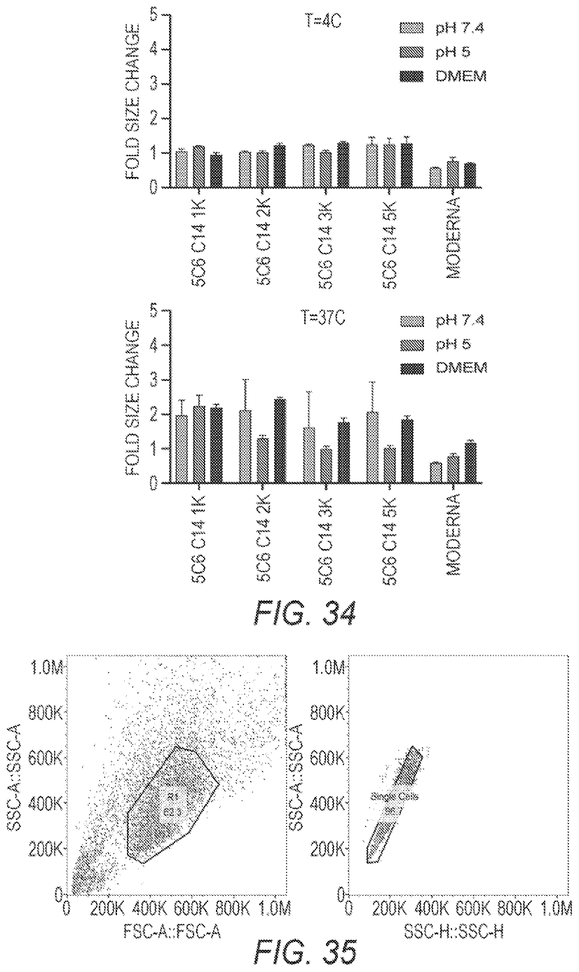

To begin the mechanism studies, the cellular association of 5C6 PNPs was investigated, Cy5-labeled FLuc mRNA was encapsulated into both 5C6 and Moderna formulations for the treatment of A549 cells. The percentage of Cy5 positive A549 cells was measured using flow cytometry after 2 and 24 hours. Here it was observed that there was no significant difference in the cellular association across 4 formulations of 5C6 PNPs, but that they all had significantly increased Cy5 positive cells when compared to the Moderna formulation (FIG. 23B, see FIG. 35 for gating strategy).

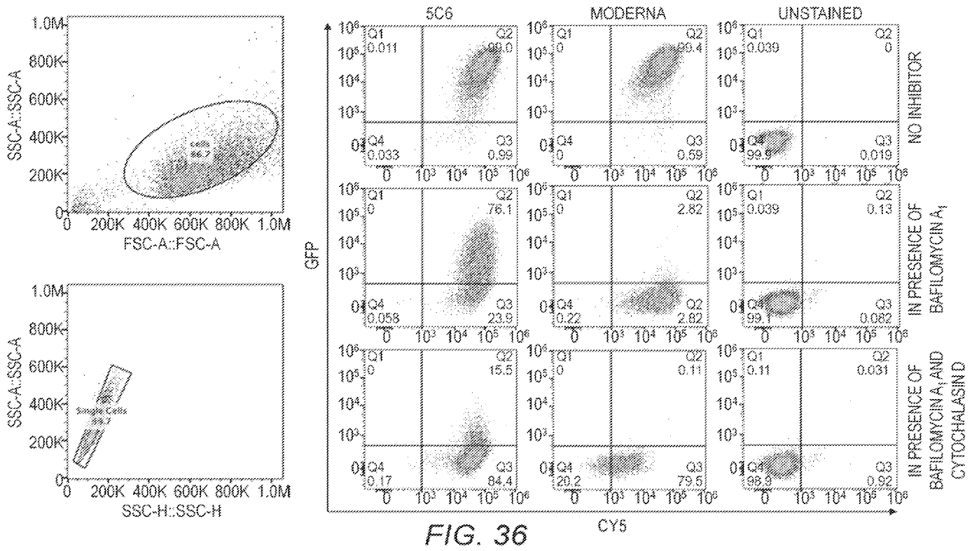

Next, the endocytosis uptake pathways of the 5C6 platform were investigated including clathrin-mediated endocytosis, caveolae-mediated endocytosis, macropinocytosis, and phagocytosis. These mechanisms can be individually inhibited by filipin, pitstop 2, EIPA, and cytochalasin D, respectively (FIG. 23C). In order to determine the contributions of each pathway in the uptake of 5C6 PNPs, A549 cells were treated with 5C6 PNPs with one of these inhibitors at a time and compared it with A549 cells treated with 5C6 PNPs but without any inhibitor. Here, it was observed the 5C6 PNPs were inhibited mostly by cytochalasin D, suggesting that phagocytosis is the main uptake pathway of these particles. Conversely, the Moderna formulation was inhibited by each endocytosis pathway inhibitor, suggesting a multifaceted uptake pathway (FIG. 23D, see FIG. 36 for gating strategy).

Thirdly, the ability of the 5C6 PNPs to escape the endosome was investigated. This mechanistic step is crucial for the mRNA cargo to reach the ribosomes where it can be translated into protein. Here, a confocal microscopy approach was employed. A549 cells were treated with Hoechst 33342 DNA stain (blue), Lysotracker (green), and nanoparticles encapsulating Cy5 labeled mRNA (red) (FIGS. 23E, 23F). The Pearson Correlation Coefficient (PCC) was then calculated to quantify the degree of overlap between the nanoparticles and the endo/lysosomes. A PCC value of 100 indicates no endosomal escape, while a PCC value of 0 indicates 100% endosomal escape. A significant enhancement was observed in endosomal escape for 5C6 PNP systems containing C14PEG 1000 and C14PEG 2000 when compared to the Moderna LNP formulation (FIG. 23G).

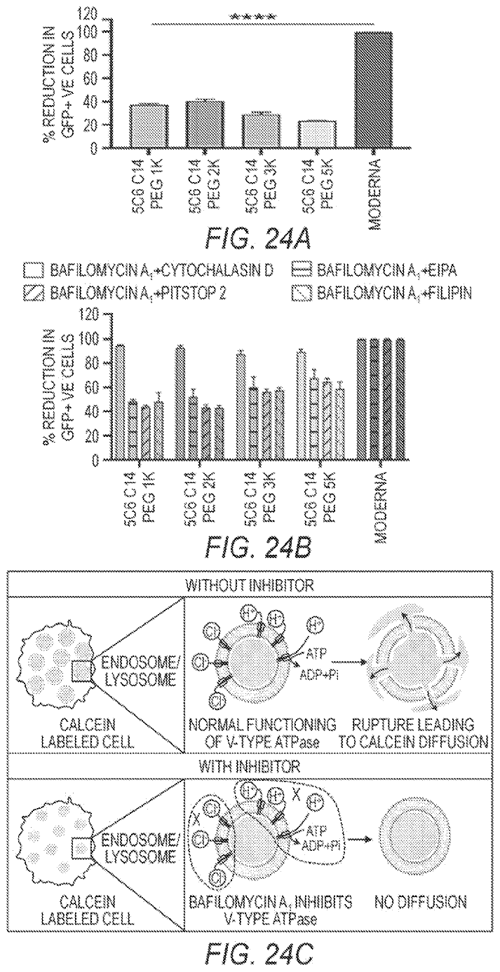

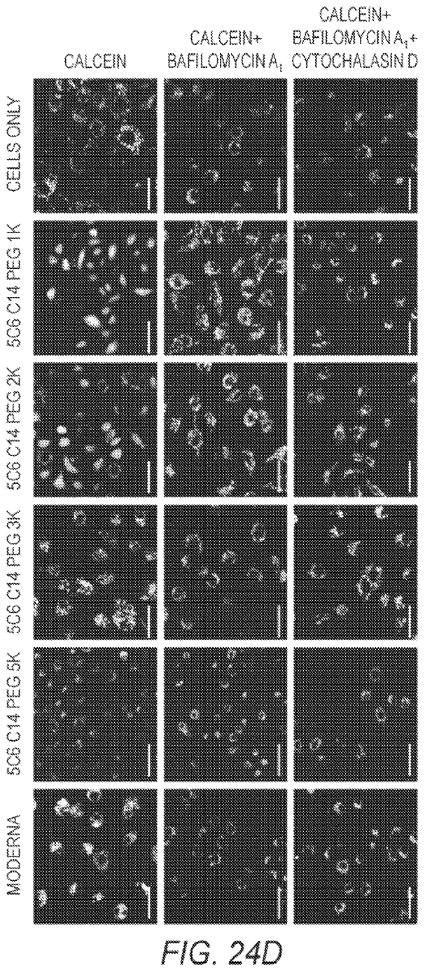

To better understand the mechanism of how the 5C6 PNPs escape from the endosome, the proton sponge effect was investigated. Here, A549 cells were treated with GFP 5C6 PNPs and bafilomycin A₁, an inhibitor of the V-ATPase proton pump, hindering the influx of protons into the endosome/lysosome that can cause rupture of the lysosome and leakage of the nanoparticles.⁵⁶⁻⁵⁷ A reduction in GFP-positive cells was seen across all 5C6 formulations, underscoring the involvement of this endosomal escape mechanism for these nanoparticles (FIG. 24A). Upon combining this inhibitor with the aforementioned endocytosis inhibitors, it was observed that the phagocytosis inhibitor combined with the proton pump inhibitor caused nearly complete loss of GFP positive cells, corroborating the use of both steps in 5C6 uptake and endosomal escape (FIG. 24B). To further corroborate the endosomal escape quantification, confocal microscopy was leveraged for a label-free calcein release assay, in which the 5C6 particles contained no fluorescent label that could potentially interfere with natural endosomal escape (FIG. 24C). Calcein is a green-colored dye that remains entrapped inside the endo/lysosomes with the nanoparticles if there is no endosomal escape. Confocal images of the different treatment groups under different conditions are depicted in FIG. 24D. Upon treatment with each formulation, calcein spread throughout the cells, indicating endosomal rupture, while the calcein in untreated cells remained localized inside the endosome. After treatment with bafilomycin A₁ (an inhibitor of proton sponge-induced endosomal rupture), calcein remained inside endosomes, serving as a negative control for endosomal escape. Additionally, treatment with bafilomycin A₁ and cytochalasin D (phagocytosis inhibitor) showed further reduced spread of calcein dye. This supports the finding that both phagocytosis and the proton sponge effect are crucial steps in the expression of mRNA delivered via 5C6 PNPs.

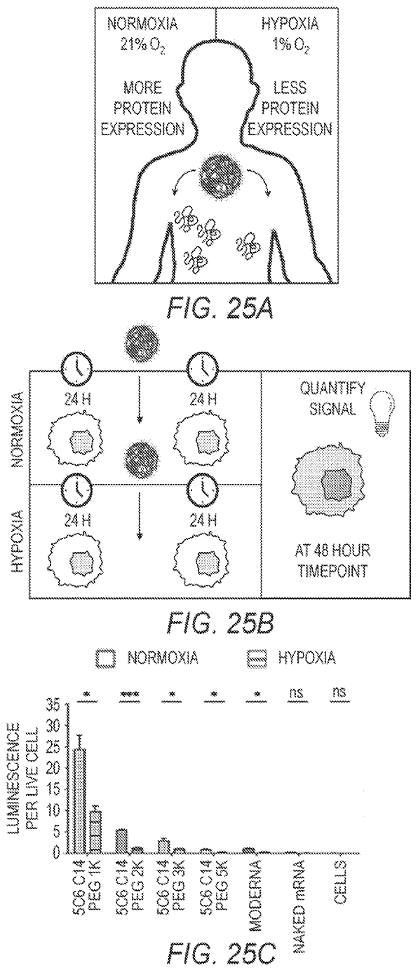

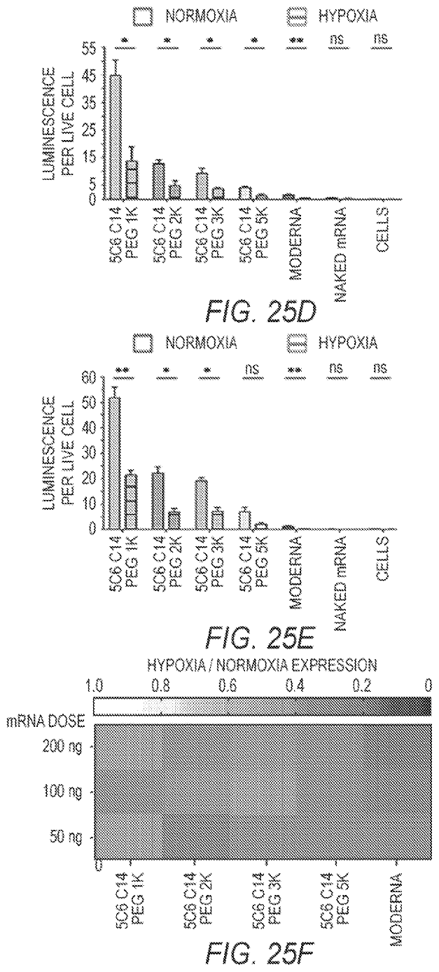

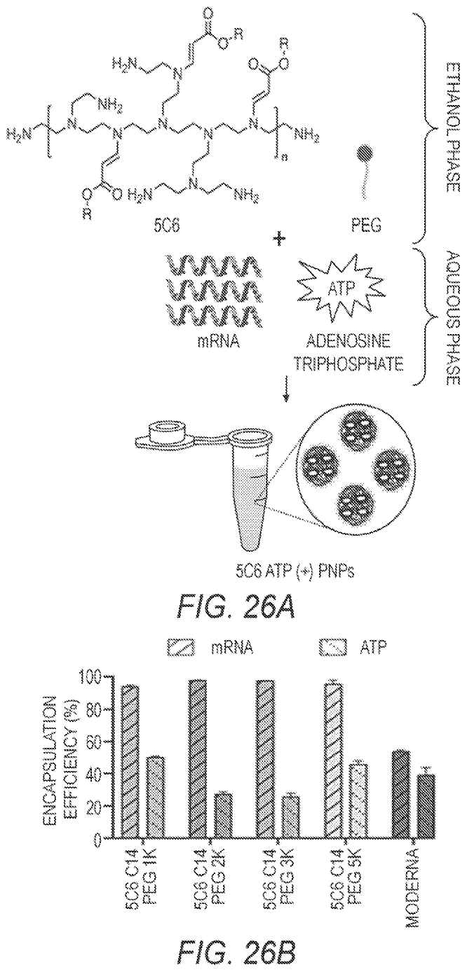

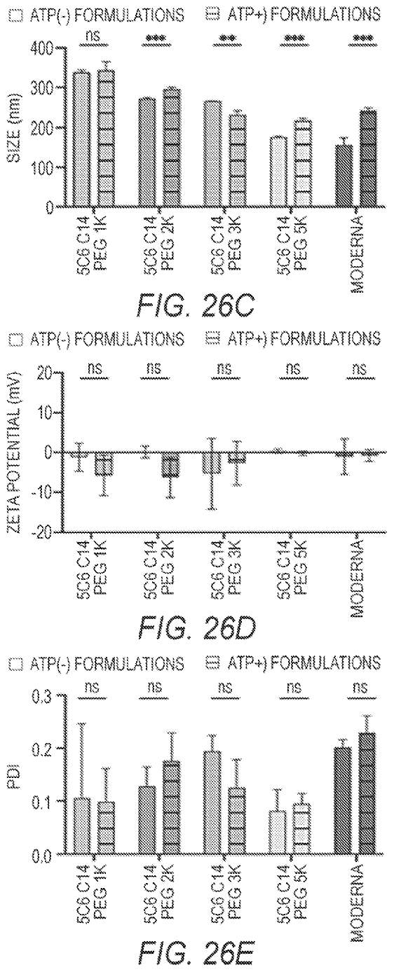

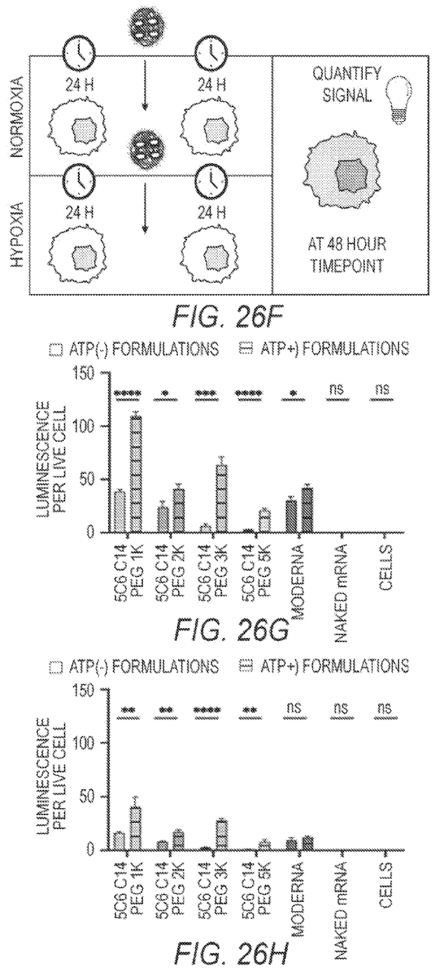

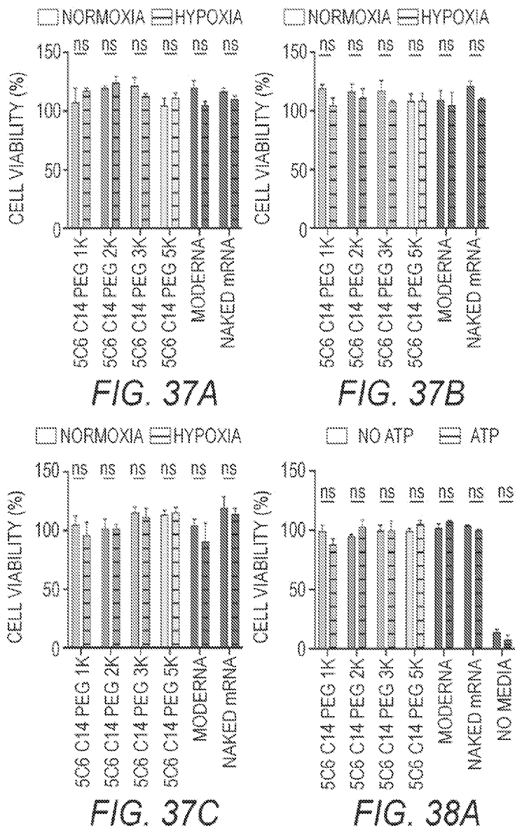

Having established an optimal formulation of 5C6 mRNA PNPs and its mechanism of action in A549 cells, the impact of hypoxia on the activity of 5C6 PNPs was next investigated. Previously, our group identified that hypoxia could reduce the efficacy of mRNA LNPs by upwards of 80%.^{20,21} Building on this, we sought to investigate whether hypoxia would also impact the efficacy of mRNA PNPs by comparing the FLuc expression levels in hypoxic vs normoxic A549 cells treated with the 5C6 PNP platform (FIGS. 25A,25B). In brief, cells were incubated under normoxia or hypoxia for 24 h and treated with 5C6 PNPs or Moderna formulation LNPs for 24 h at 50, 100, or 200 ng of FLuc mRNA. The FLuc signal was quantified for each treatment under normoxia and hypoxia (FIGS. 25C-25E). The ratio between the FLuc signal in normoxic and hypoxic conditions is shown in a heat map to illustrate the universal reduction in signal under hypoxia (FIG. 25F). This reduction did not appear due to decreased cell viability (FIG. 37). Our laboratory's prior research has established that incorporating ATP into mRNA LNPs significantly enhances their efficacy, even under hypoxia.²⁰ Informed by this, ATP was incorporated into 5C6, with ATP encapsulation efficiency ranging from 25% to 50% for evaluation in both normoxic and hypoxic conditions (FIGS. 26A-26B, 26F). Such addition of ATP, while not significantly affecting the particle characteristics or tolerability (FIGS. 26C-26E, 38), increased the overall mRNA expression across all formulations under both hypoxic and normoxic conditions (FIGS. 26G-26H). Indeed, among the high molecular weight PEG formulations, the mRNA expression increased up to 10-fold. This increased efficacy makes this 5C6 platform a promising mRNA delivery system tunable for hypoxia-associated diseases.

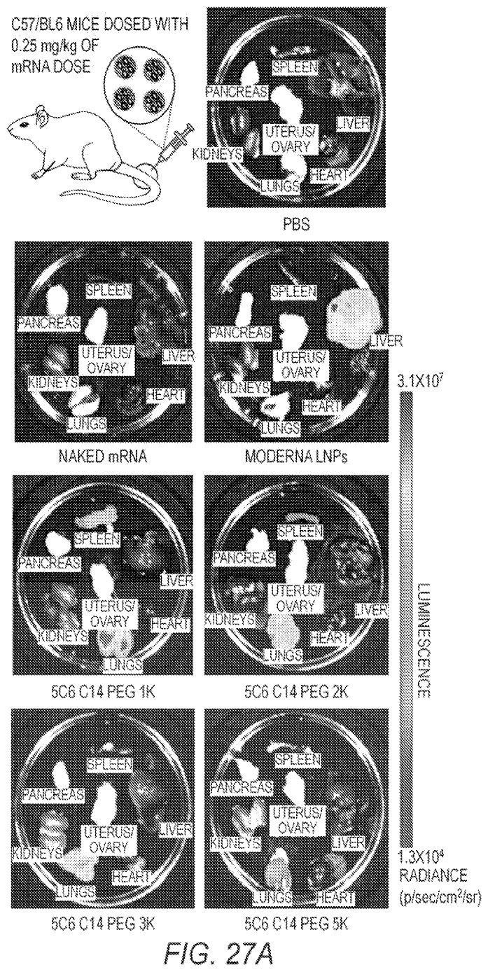

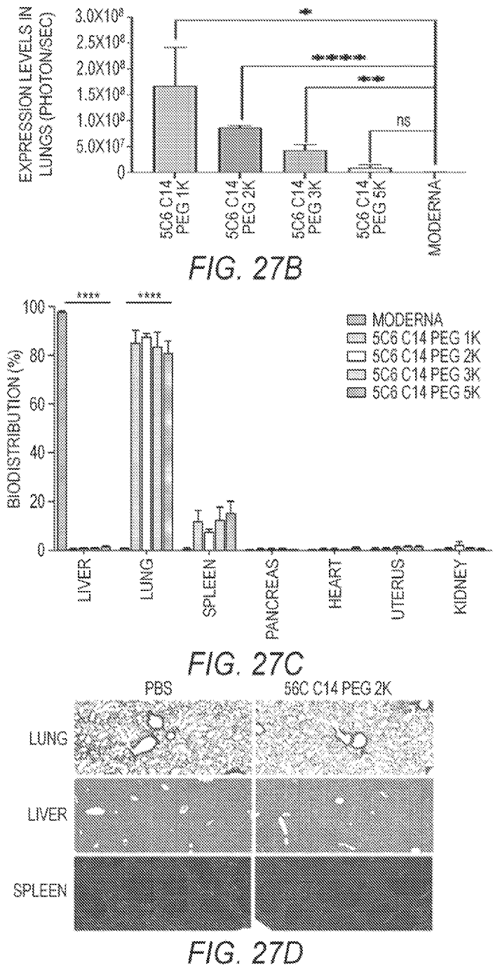

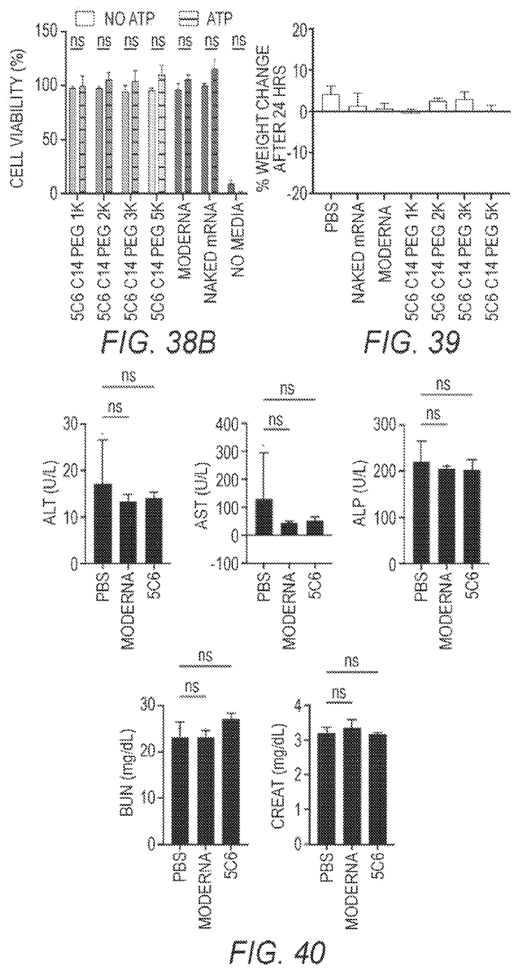

Finally, the study was concluded by investigating the efficacy, tolerability, and biodistribution of 5C6 in vivo. All formulations encapsulating FLuc mRNA were intravenously administered to a cohort of healthy C57BL/6 mice (n=3) at 0.25 mg overall mRNA dose per kg weight of mouse. After 24 h, the FLuc expression was quantified using IVIS. As controls and benchmarks, the formulations were compared side-by-side with phosphate-buffered saline, naked FLuc mRNA, and an analogous Moderna LNP formulation. In collectively analyzing these data, several trends emerged. Firstly, 5C6 formulations caused mRNA expression primarily in the lungs, while the Moderna formulation yielded mRNA expression primarily in the liver, highlighting the ability of this 5C6 system to deliver mRNA to organs that cannot be reached by commercial systems (FIGS. 27A-27C). Secondly, the levels of FLuc expression in the lungs mirrored the trends observed in the in vitro analyses, where a decrease in the molecular weight of the PEG was associated with reduced expression levels. Thirdly, no toxicity or tolerability issues were observed in either weight changes (FIG. 39) or histological analyses (FIG. 27D) after treatment with 5C6 formulations.

### CONCLUSION

In this work, 5C6 was developed, a polymer designed for efficient mRNA delivery to the lungs. Subsequent optimizations of the formulation, particularly involving the identification of an optimal polyethylene glycol (PEG), led to the development of a stable and highly efficacious particle with promising mRNA delivery capabilities. After optimizing for PEG, formulations were also optimized for better performance in hypoxic conditions by the addition of ATP. Their uptake and endosomal escape were then also studied to further understand how they were different than lipid nanoparticles. In vivo experiments in mice highlighted a lung-centric distribution of mRNA with the formulations, signifying their potential for targeted lung delivery. The formulations were also well-tolerated in vivo.

Going forward, the 5C6 system can be characterized for its delivery properties across more oxygenation levels and in applications for specific diseases affecting the lungs. While mRNA delivery platforms have seen considerable success in the clinic, there remains a significant hurdle for mRNA delivery to extrahepatic tissues as well as those that are not oxygenated at traditional cell culture levels. These findings underscore the efficacy of these formulations for lung-targeted mRNA delivery, emphasizing their potential in addressing diseases characterized by low oxygen levels such as lung cancer. Additionally, this work underscores the importance and feasibility of developing new nanoparticle formulations that can overcome hurdles faced by many drug delivery systems.

The following references are pertinent to Example 5:
(1) Fenton, O. S.; Olafson, K. N.; Pillai, P. S.; Mitchell, M. J.; Langer, R. Advances in Biomaterials for Drug Delivery. Advanced Materials 2018, 30 (29), 1705328. https://doi.org/10.1002/adma.201705328.
(2) Lee, S. M.; Cheng, Q.; Yu, X.; Liu, S.; Johnson, L. T.; Siegwart, D. J. A Systematic Study of Unsaturation in Lipid Nanoparticles Leads to Improved mRNA Transfection In Vivo. Angewandte Chemie 2021, 133 (11), 5912-5917. https://doi.org/10.1002/ange.202013927.
(3) Shepherd, S. J.; Issadore, D.; Mitchell, M. J. Microfluidic Formulation of Nanoparticles for Biomedical Applications. Biomaterials 2021, 274, 120826. https://doi.org/10.1016/j.biomaterials.2021.120826.
(4) Bhat, B.; Karve, S.; Anderson, D. G. mRNA Therapeutics: Beyond Vaccine Applications. Trends in Molecular Medicine 2021, 27 (9), 923-924. https://doi.org/10.1016/j.molmed.2021.05.004.
(5) Rohner, E.; Yang, R.; Foo, K. S.; Goedel, A.; Chien, K. R. Unlocking the Promise of mRNA Therapeutics. Nat Biotechnol 2022, 40 (11), 1586-1600. https://doi.org/10.1038/s41587-022-01491-z.
(6) VanKeulen-Miller, R.; Fenton, O. S. Messenger RNA Therapy for Female Reproductive Health. Mol. Pharmaceutics 2024, 21 (2), 393-409. https://doi.org/10.1021/acs.molpharmaceut.3c00803.
(7) Tilstra, G.; Couture-Senecal, J.; Lau, Y. M. A.; Manning, A. M.; Wong, D. S. M.; Janaeska, W. W.; Wuraola, T. A.; Pang, J.; Khan, O. F. Iterative Design of Ionizable Lipids for Intramuscular mRNA Delivery. J Am. Chem. Soc. 2023, 145 (4), 2294-2304. https://doi.org/10.1021/jacs.2c10670.
(8) Witzigmann, D.; Kulkarni, J. A.; Leung, J.; Chen, S.; Cullis, P. R.; van der Meel, R. Lipid Nanoparticle Technology for Therapeutic Gene Regulation in the Liver. Adv Drug Deliv Rev 2020, 159, 344-363. https://doi.org/10.1016/j.addr.2020.06.026.
(9) Rui, Y.; Wilson, D. R.; Tzeng, S. Y.; Yamagata, H. M.; Sudhakar, D.; Conge, M.; Berlinicke, C. A.; Zack, D. J.; Tuesca, A.; Green, J. J. High-Throughput and High-Content Bioassay Enables Tuning of Polyester Nanoparticles for Cellular Uptake, Endosomal Escape, and Systemic in Vivo Delivery of mRNA. Science Advances 2022, 8 (1), eabk2855. https://doi.org/10.1126/sciadv.abk2855.
(10) Reichmuth, A. M.; Oberli, M. A.; Jaklenec, A.; Langer, R.; Blankschtein, D. mRNA Vaccine Delivery Using Lipid Nanoparticles. Ther Deliv 2016, 7 (5), 319-334. https://doi.org/10.4155/tde-2016-0006.
(11) Eygeris, Y.; Gupta, M.; Kim, J.; Jozic, A.; Gautam, M.; Renner, J.; Nelson, D.; Bloom, E.; Tuttle, A.; Stoddard, J.; Reynaga, R.; Neuringer, M.; Lauer, A. K.; Ryals, R. C.; Sahay, G. Thiophene-Based Lipids for mRNA Delivery to Pulmonary and Retinal Tissues. Proceedings of the National Academy of Sciences 2024, 121 (11), e2307813 120. https://doi.org/10.1073/pnas.2307813120.
(12) Kim, J.; Eygeris, Y.; Ryals, R. C.; Jozid, A.; Sahay, G. Strategies for Non-Viral Vectors Targeting Organs beyond the Liver. Nat. Nanotechnol. 2023. https://doi.org/10.1038/s41565-023-01563-4.
(13) Billingsley, M. M.; Gong, N.; Mukalel, A. J.; Thatte, A. S.; El-Mayta, R.; Patel, S. K.; Metzloff, A. E.; Swingle, K. L.; Han, X.; Xue, L.; Hamilton, A. G., Safford, H. C.; Alameh, M.-G.; Papp, T. E.; Parhiz, H.; Weissman, D.; Mitchell, M. J. In Vivo mRNA CAR T Cell Engineering via Targeted Ionizable Lipid Nanoparticles with Extrahepatic Tropism. Small 2024, 20 (11), 2304378. https://doi.org/10.1002/smll.202304378.
(14) Hou, X.; Zaks, T.; Langer, R.; Dong, Y. Lipid Nanoparticles for mRNA Delivery. Nat Rev Mater 2021, 6 (12), 1078-1094. https://doi.org/10.1038/s41578-021-00358-0.
(15) van Haasteren, J.; Li, J.; Scheideler, O. J.; Murthy, N.; Schaffer, D. V. The Delivery Challenge: Fulfilling the Promise of Therapeutic Genome Editing. Nat Biotechnol 2020, 38 (7), 845-855. https://doi.org/10.1038/s41587-020-0565-5.
(16) Fenton, O. S.; Kauffman, K. J.; Kaczmarek, J. C.; McClellan, R. L.; Jhunjhunwala, S.; Tibbitt, M. W.; Zeng, M. D.; Appel, E. A.; Dorkin, J. R.; Mir, F. F.; Yang, J. H.; Oberli, M. A.; Heartlein, M. W.; DeRosa, F.; Langer, R.; Anderson, D. G. Synthesis and Biological Evaluation of Ionizable Lipid Materials for the In Vivo Delivery of Messenger RNA to B Lymphocytes. Advanced Materials 2017, 29 (33), 1606944. https://doi.org/10.1002/adma.201606944.
(17) Montgomery, S. T.; Mall, M. A.; Kicic, A.; Stick, S. M. Hypoxia and Sterile Inflammation in Cystic Fibrosis Airways: Mechanisms and Potential Therapies. Eur Respir J 2017, 49 (1), 1600903. https://doi.org/10.1183/13993003.00903-2016.
(18) Walsh, J. C.; Lebedev, A.; Aten, E.; Madsen, K.; Marciano, L.; Kolb, H. C. The Clinical Importance of Assessing Tumor Hypoxia: Relationship of Tumor Hypoxia to Prognosis and Therapeutic Opportunities. Antioxidants & Redox Signaling 2014, 21 (10), 1516-1554. https://doi.org/10.1089/ars.2013.5378.
(19) Steinke, J. W.; Woodard, C. R.; Borish, L. Role of Hypoxia in Inflammatory Upper Airway Disease. Current Opinion in Allergy & Clinical Immunology 2008, 8 (1), 16-20. https://doi.org/10.1097/ACI.0b013e3282f3f488.
(20) Ma, Y.; Fenton, O. S. An Efficacy and Mechanism Driven Study on the Impact of Hypoxia on Lipid Nanoparticle Mediated mRNA Delivery. J. Am. Chem. Soc. 2023, 145
(20), 11375-11386. https://doi.org/10.1021/jacs.3c02584.
(21) Ma, Y.; Fenton, O. S. A Unified Strategy to Improve Lipid Nanoparticle Mediated mRNA Delivery Using Adenosine Triphosphate. J. Am. Chem. Soc. 2023, 145 (36), 19800-19811. https://doi.org/10.1021/jacs.3c05574.
(22) Li, B.; Manan, R. S.; Liang, S.-Q.; Gordon, A.; Jiang, A.; Varley, A.; Gao, G.; Langer, R.; Xue, W.; Anderson, D. Combinatorial Design of Nanoparticles for Pulmonary mRNA Delivery and Genome Editing. Nat Biotechnol 2023, 1-6. https://doi.org/10.1038/s41587-023-01679-x.
(23) Neill, B.; Romero, A. R.; Fenton, O. S. Advances in Nonviral mRNA Delivery Materials and Their Application as Vaccines for Melanoma Therapy. ACS Appl. Bio Mater. 2023. https://doi.org/10.1021/acsabm.3c00721.
(24) Kaczmarek, J. C.; Kauffman, K. J.; Fenton, O. S.; Sadtler, K.; Patel, A. K.; Heartlein, M. W.; DeRosa, F.; Anderson, D. G. Optimization of a Degradable Polymer-Lipid Nanoparticle for Potent Systemic Delivery of mRNA to the Lung Endothelium and Immune Cells. Nano Lett. 2018, 18 (10), 6449-6454.
https://doi.org/10.1021/acs.nanolett.8b02917.
(25) Pack, D. W.; Hoffman, A. S.; Pun, S.; Stayton, P. S. Design and Development of Polymers for Gene Delivery. Nat Rev Drug Discov 2005, 4 (7), 581-593. https://doi.org/10.1038/nrd1775.
(26) Davis, M.; Pun, S.; Bellocq, N.; Reineke, T.; Popielarski, S.; Mishra, S.; Heidel, J. Self-Assembling Nucleic Acid Delivery Vehicles via Linear, Water-Soluble, Cyclodextrin-Containing Polymers. CMC 2004, 11 (2), 179-197. https://doi.org/10.2174/0929867043456179.
(27) Suberi, A.; Grun, M. K.; Mao, T.; Israelow, B.; Reschke, M.; Grundler, J.; Akhtar, L.; Lee, T.; Shin, K.; Piotrowski-Daspit, A. S.; Homer, R. J.; Iwasaki, A.; Suh, H.-W.; Saltzman, W. M. Polymer Nanoparticles Deliver mRNA to the Lung for Mucosal Vaccination. Science Translational Medicine 2023, 15 (709), eabq0603. https://doi.org/10.1126/scitranslmed.abq0603.
(28) Kaczmarek, J. C.; Patel, A. K.; Kauffman, K. J.; Fenton, O. S.; Webber, M. J.; Heartlein, M. W.; DeRosa, F.; Anderson, D. G. Polymer-Lipid Nanoparticles for Systemic Delivery of mRNA to the Lungs. Angew Chem Int Ed Engl 2016, 55 (44), 13808-13812. https://doi.org/10.1002/anie.201608450.
(29) Dahlman, J. E.; Barnes, C.; Khan, O.; Thiriot, A.; Jhunjunwala, S.; Shaw, T. E.; Xing, Y.; Sager, H. B.; Sahay, G.; Speciner, L.; Bader, A.; Bogorad, R. L.; Yin, H.; Racie, T.; Dong, Y.; Jiang, S.; Seedorf, D.; Dave, A.; Sandu, K. S.; Webber, M. J.; Novobrantseva, T.; Ruda, V. M.; Lytton-Jean, A. K. R.; Levins, C. G.; Kalish, B.; Mudge, D. K.; Perez, M.; Abezgauz, L.; Dutta, P.; Smith, L.; Charisse, K.; Kieran, M. W.; Fitzgerald, K.; Nahrendorf, M.; Danino, D.; Tuder, R. M.; von Andrian, U. H.; Akinc, A.; Schroeder, A.; Panigrahy, D.; Kotelianski, V.; Langer, R.; Anderson, D. G. In Vivo Endothelial siRNA Delivery Using Polymeric Nanoparticles with Low Molecular Weight. Nat Nanotechnol 2014, 9 (8), 648-655. https://doi.org/10.1038/nnano.2014.84.
(30) Patel, A. K.; Kaczmarek, J. C.; Bose, S.; Kauffman, K. J.; Mir, F.; Heartlein, M. W.; DeRosa, F.; Langer, R.; Anderson, D. G. Inhaled Nanoformulated mRNA Polyplexes for Protein Production in Lung Epithelium. Advanced Materials 2019, 31 (8), 1805116. https://doi.org/10.1002/adma.201805116.
(31) Lokugamage, M. P.; Vanover, D.; Beyersdorf, J.; Hatit, M. Z. C.; Rotolo, L.; Echeverri, E. S.; Peck, H. E.; Ni, H.; Yoon, J.-K.; Kim, Y.; Santangelo, P. J.; Dahlman, J. E. Optimization of Lipid Nanoparticles for the Delivery of Nebulized Therapeutic mRNA to the Lungs. Nat Biomed Eng 2021, 5 (9), 1059-1068. https://doi.org/10.1038/s41551-021-00786-x.
(32) Chaudhary, N.; Weissman, D.; Whitehead, K. A. mRNA Vaccines for Infectious Diseases: Principles, Delivery and Clinical Translation. Nat Rev Drug Discov 2021, 20 (11), 817-838. https://doi.org/10.1038/s41573-021-00283-5.
(33) Ulkoski, D.; Bak, A.; Wilson, J. T.; Krishnamurthy, V. R. Recent Advances in Polymeric Materials for the Delivery of RNA Therapeutics. Expert Opin Drug Deliv 2019, 16 (11), 1149-1167. https://doi.org/10.1080/17425247.2019.1663822.
(34) Blakney, A. K.; Yilmaz, G.; McKay, P. F.; Becer, C. R.; Shattock, R. J. One Size Does Not Fit All: The Effect of Chain Length and Charge Density of Poly(Ethylene Imine) Based Copolymers on Delivery of pDNA, mRNA, and RepRNA Polyplexes. Biomacromolecules 2018, 19 (7), 2870-2879. https://doi.org/10.1021/acs.biomac.8b00429.
(35) Jorgensen, A. M.; Wibel, R.; Bernkop-Schnirch, A. Biodegradable Cationic and Ionizable Cationic Lipids: A Roadmap for Safer Pharmaceutical Excipients. Small 2023, 19 (17), 2206968. https://doi.org/10.1002/smll.202206968.
(36) Ben-Akiva, E.; Karlsson, J.; Hemmati, S.; Yu, H.; Tzeng, S. Y.; Pardoll, D. M.; Green, J. J. Biodegradable Lipophilic Polymeric mRNA Nanoparticles for Ligand-Free Targeting of Splenic Dendritic Cells for Cancer Vaccination. Proceedings of the National Academy of Sciences 2023, 120 (26), e2301606120. https://doi.org/10.1073/pnas.2301606120.
(37) Whitehead, K. A.; Dorkin, J. R.; Vegas, A. J.; Chang, P. H.; Veiseh, O.; Matthews, J.; Fenton, O. S.; Zhang, Y.; Olejnik, K. T.; Yesilyurt, V.; Chen, D.; Barros, S.; Klebanov, B.; Novobrantseva, T.; Langer, R.; Anderson, D. G. Degradable Lipid Nanoparticles with Predictable in Vivo siRNA Delivery Activity. Nat Commun 2014, 5 (1), 4277. https://doi.org/10.1038/ncomms5277.
(38) Han, X.; Zhang, H.; Butowska, K.; Swingle, K. L.; Alameh, M.-G.; Weissman, D.; Mitchell, M. J. An Ionizable Lipid Toolbox for RNA Delivery. Nat Commun 2021, 12 (1), 7233. https://doi.org/10.1038/s41467-021-27493-0.
(39) Tanaka, H.; Takahashi, T.; Konishi, M.; Takata, N.; Gomi, M.; Shirane, D.; Miyama, R.; Hagiwara, S.; Yamasaki, Y.; Sakurai, Y.; Ueda, K.; Higashi, K.; Moribe, K.; Shinsho, E.; Nishida, R.; Fukuzawa, K.; Yonemochi, E.; Okuwaki, K.; Mochizuki, Y.; Nakai, Y.; Tange, K.; Yoshioka, H.; Tamagawa, S.; Akita, H. Self-Degradable Lipid-Like Materials Based on “Hydrolysis Accelerated by the Intra-Particle Enrichment of Reactant (HyPER)” for Messenger RNA Delivery. Advanced Functional Materials 2020, 30 (34), 1910575. https://doi.org/10.1002/adfm.201910575.
(40) Liu, J.; Chang, J.; Jiang, Y.; Meng, X.; Sun, T.; Mao, L.; Xu, Q.; Wang, M. Fast and Efficient CRISPR/Cas9 Genome Editing In Vivo Enabled by Bioreducible Lipid and Messenger RNA Nanoparticles. Advanced Materials 2019, 31 (33), 1902575. https://doi.org/10.1002/adma.201902575.
(41) Ma, F.; Yang, L.; Sun, Z.; Chen, J.; Rui, X.; Glass, Z.; Xu, Q. Neurotransmitter-Derived Lipidoids (NT-Lipidoids) for Enhanced Brain Delivery through Intravenous Injection. Science Advances 2020, 6 (30), eabb4429. https://doi.org/10.1126/sciadv.abb4429.
(42) Zhao, X.; Glass, Z.; Chen, J.; Yang, L.; Kaplan, D. L.; Xu, Q. mRNA Delivery Using Bioreducible Lipidoid Nanoparticles Facilitates Neural Differentiation of Human Mesenchymal Stem Cells. Advanced Healthcare Materials 2021, 10 (4), 2000938. https://doi.org/10.1002/adhm.202000938.
(43) Reul, R.; Nguyen, J.; Kissel, T. Amine-Modified Hyperbranched Polyesters as Non-Toxic, Biodegradable Gene Delivery Systems. Biomaterials 2009, 30 (29), 5815-5824. https://doi.org/10.1016/j.biomaterials.2009.06.057.
(44) Hashiba, K.; Sato, Y.; Taguchi, M.; Sakamoto, S.; Otsu, A.; Maeda, Y.; Shishido, T.; Murakawa, M.; Okazaki, A.; Harashima, H. Branching Ionizable Lipids Can Enhance the Stability, Fusogenicity, and Functional Delivery of mRNA. Small Science 2023, 3 (1), 2200071. https://doi.org/10.1002/smsc.202200071.
(45) Zhang, Y.; Sun, C.; Wang, C.; Jankovic, K. E.; Dong, Y. Lipids and Lipid Derivatives for RNA Delivery. Chem. Rev. 2021, 121 (20), 12181-12277. https://doi.org/10.1021/acs.chemrev.1c00244.
(46) Ponti, F.; Campolungo, M.; Melchiori, C.; Bono, N.; Candiani, G. Cationic Lipids for Gene Delivery: Many Players, One Goal. Chemistry and Physics of Lipids 2021, 235, 105032. https://doi.org/10.1016/j.chemphyslip.2020.105032.
(47) Eygeris, Y.; Gupta, M.; Kim, J.; Sahay, G. Chemistry of Lipid Nanoparticles for RNA Delivery. Acc. Chem. Res. 2022, 55 (1), 2-12.
https://doi.org/10.1021/acs.accounts.1c00544.
(48) Miao, L.; Zhang, Y.; Huang, L. mRNA Vaccine for Cancer Immunotherapy. Mol Cancer 2021, 20 (1), 41. https://doi.org/10.1186/s12943-021-01335-5.
(49) Ziebarth, J. D.; Kennetz, D. R.; Walker, N. J.; Wang, Y. Structural Comparisons of PEI/DNA and PEI/siRNA Complexes Revealed with Molecular Dynamics Simulations. J Phys Chem B 2017, 121 (8), 1941-1952. https://doi.org/10.1021/acs.jpcb.6b10775.
(50) Helmschrodt, C.; Höbel, S.; Schöniger, S.; Bauer, A.; Bonicelli, J.; Gringmuth, M.; Fietz, S. A.; Aigner, A.; Richter, A.; Richter, F. Polyethylenimine Nanoparticle-Mediated siRNA Delivery to Reduce α-Synuclein Expression in a Model of Parkinson's Disease. Mol Ther Nucleic Acids 2017, 9, 57-68. https://doi.org/10.1016/j.omtn.2017.08.013.
(51) Wen, Y.; Pan, S.; Luo, X.; Zhang, X.; Zhang, W.; Feng, M. A Biodegradable Low Molecular Weight Polyethylenimine Derivative as Low Toxicity and Efficient Gene Vector. Bioconjug Chem 2009, 20 (2), 322-332. https://doi.org/10.1021/bc800428y.
(52) Hall, A.; Lachelt, U.; Bartek, J.; Wagner, E.; Moghimi, S. M. Polyplex Evolution: Understanding Biology, Optimizing Performance. Mol Ther 2017, 25 (7), 1476-1490. https://doi.org/10.1016/j.ymthe.2017.01.024.
(53) Ke, X.; Shelton, L.; Hu, Y.; Zhu, Y.; Chow, E.; Tang, H.; Santos, J. L.; Mao, H.-Q. Surface-Functionalized PEGylated Nanoparticles Deliver Messenger RNA to Pulmonary Immune Cells. ACS Appl. Mater. Interfaces 2020, 12 (32), 35835-35844. https://doi.org/10.1021/acsami.0c08268.
(54) Chen, S.; Jin, T. Poly-Cross-Linked PEI Through Aromatically Conjugated Imine Linkages as a New Class of pH-Responsive Nucleic Acids Packing Cationic Polymers. Frontiers in Pharmacology 2016, 7.
(55) Kursa, M.; Walker, G. F.; Roessler, V.; Ogris, M.; Roedl, W.; Kircheis, R.; Wagner, E. Novel Shielded Transferrin-Polyethylene Glycol-Polyethylenimine/DNA Complexes for Systemic Tumor-Targeted Gene Transfer. Bioconjug Chem 2003, 14 (1), 222-231. https://doi.org/10.1021/bc0256087.
(56) Wojnilowicz, M.; Glab, A.; Bertucci, A.; Caruso, F.; Cavalieri, F. Super-Resolution Imaging of Proton Sponge-Triggered Rupture of Endosomes and Cytosolic Release of Small Interfering RNA. ACS Nano 2019, 13 (1), 187-202. https://doi.org/10.1021/acsnano.8b05151.
(57) Kichler, A.; Leborgne, C.; Coeytaux, E.; Danos, O. Polyethylenimine-Mediated Gene Delivery: A Mechanistic Study. J Gene Med. 2001, 3 (2), 135-144. https://doi.org/10.1002/jgm.173.

### Experiments And Methods

### 1.1. Materials

Polyethylenimine (Mn ~600 by GPC), cholesterol, phosphate-buffered saline (PBS, pH 7.4), sodium citrate, citrate acid, Dulbecco's phosphate-buffered saline (DPBS), penicillin-streptomycin, ethanol, Triton X-100, heparin (>150 USP), Chloroform-D, acetonitrile, filipin from S. filipinensis, cytochalasin D, hexanol, toluene, dichloromethane, propiolic acid and p-toluenesulfonic acid, Adenosine 5′-triphosphate (ATP) disodium salt hydrate, 6-(p-toluidino)-2-naphthalenesulfonic acid were purchased from Sigma-Aldrich. 1,2-distearoyl-sn-glycero-3-phosphocholine (DSPC), 1,2-dimyristoyl-sn-glycero-3-phosphoethanolamine-N-[methoxy-(polyethyleneglycol)-1000](ammonium salt) (C14-PEG-1000), 1,2-dimyristoyl-sn-glycero-3-phosphoethanolamine-N-[methoxy-(polyethyleneglycol)-2000](ammonium salt) (C14-PEG-2000), 1,2-dimyristoyl-sn-glycero-3-phosphoethanolamine-N-[methoxy-(polyethyleneglycol)-3000](ammonium salt) (C14-PEG-3000) and 1,2-dimyristoyl-sn-glycero-3-phosphoethanolamine-N-[methoxy-(polyethyleneglycol)-5000](ammonium salt) (C₁₄-PEG-5000) were purchased from Avanti. Moderna SM-102 Lipid was bought from Broadpharm. Pitstop 2 was purchased from Abcam. Dulbecco's modified Eagle's medium (DMEM), Fetal Bovine Serum (FBS), Hoechst 33342 and calcein were purchased from Life Technologies. Firefly luciferase mRNA was obtained from TriLink Biotechnologies and Cy5 labelled Firefly luciferase (FLuc) mRNA was obtained from ApexBio. Quant-iT RiboGreen RNA assay kit, LysoTracker Green DND-26, bafilomycin A₁, ATP determination kit was purchased from Thermo Fisher Scientific. Bright-Glo Luciferase assay system and VivoGlo Luciferin were obtained from Promega. Ultrapure water (Milli-Q) with a resistivity greater than 18.2 MΩ cm was used in all experiments and obtained from a three stage Millipore Milli-Q Plus 185 purification system. All chemicals were used without further purification.

### 1.2 Synthesis and Material Characterization

For the synthesis of C6 alkynoate, hexanol (5 mmol) was refluxed under dean-stark conditions with propiolic acid (5 mmol) in the presence of para-toluenesulfonic acid (0.5 mmol) in toluene (0.167 M). The alkynoate was then purified using silica gel column chromatography.

For the synthesis of 5C6, PEI (0.06 mmol) and C6 alkynoate (0.3 mmol) were added in a in dichloromethane (0.015 M) in a 2-dram scintillation vial and stirred for 3 days at room temperature and was then concentrated using high pressure vacuum.

NMR samples were prepared in chloroform-D solvent and analyzed on 400 MHz Bruker NMR instrument. All mass spec samples were prepared in acetonitrile and analyzed on Q-Exactive HF-X Hybrid Quadrupole-Orbitrap Mass Spectrometer.

### 1.3 Nanoparticle Synthesis

An organic phase was generated by dissolving a mixture of the synthesized polymer-lipid and 1,2-dimyristoyl-sn-glycero-3-phosphoethanolamine-N-[methoxy-(polyethyleneglycol)-1000](ammonium salt) (C14-PEG-1000) or 1,2-dimyristoyl-sn-glycero-3-phosphoethanolamine-N-[methoxy-(polyethyleneglycol)-2000](ammonium salt) (C14-PEG-2000) or 1,2-dimyristoyl-sn-glycero-3-phosphoethanolamine-N-[methoxy-(polyethyleneglycol)-3000](ammonium salt) (C14-PEG-3000) or and 1,2-dimyristoyl-sn-glycero-3-phosphoethanolamine-N-[methoxy-(polyethyleneglycol)-5000](ammonium salt) (C14-PEG-5000) at molar ratio of 99.75 to 0.25 in ethanol for all the experiments except for the experiments. The polymer-lipid to mRNA weight ratio was 10:1 for all formulations which corresponds to N/P ratio of 58. All the ethanol stock solutions were prepared at concentration of 10 mg/mL. The aqueous phase was prepared with 10 mM pH 3 citrate buffer with mRNA. mRNAs were stored at −80° C. and thawed prior to use. The ethanol phase and aqueous phase were hand mixed at 1:3 volume ratio. Resultant nanoparticles were dialyzed against 1×PBS in a 20 k MWCO dialysis cassette at 4° C. for 2 h and were stored at 4° C. prior to use.

### 1.3.1 Estimating N/P Ratio

Determining the moles of nitrogen residues in 5C6 polymer is an approximation, since the polymers are polydisperse.

### 1.3.1.2 Determining Moles of Phosphate

Each μg of oligonucleotide contains 3 nmol of negatively charged phosphate. Thus, 40 μg of mRNA would have 120 nmol of negatively charged phosphate, as would 40 μg of DNA.

### 1.3.1.3 Determining the Moles of Nitrogen

The number average molecular weight of the polymer (Mn) is obtained using a polymer analysis software called Polymerix (709.341 Da). It was assumed that the moles of nitrogen on 5C6 will be same as the number of moles in PEI starting material. The mass of the PEI was then calculated corresponding to the moles of 5C6 used. This mass was then used to calculate moles of nitrogen by dividing it with 43.04 g/mol (protonable unit of PEI, i.e one mole of N in one monomer of PEI).

### 1.3.1.4 Calculating the N/P Ratio

To calculate the N/P ratio, one only need divide the mole of ionizable nitrogen residues in the 5C6/nucleic acid complex by the moles of negatively charged phosphates to obtain the N/P ratio. For example, if 500 nmol of 5C6 (6970 nmol of nitrogen residues) are complexed with 40 μg of mRNA (120 nmol of negatively charged phosphate), then the N/P ratio for the particles will be 58.

### 1.3.2 Formulations Containing ATP

For the formulation of ATP (+) formulation: the ethanol phase was prepared with the same molar ratio summarized in FIG. 2c. Aqueous phase was made by dissolving the 0.26 mg mL¹ of mRNA and various ATP concentration (2.0 mg/mL) of a volume ratio of 1:1 in 10 mM citrate buffer (pH 3). Finally, the synthesized ATP (+) formulations were dialyzed against 1×PBS in a 20,000 MWCO cassette at 4° C. cold room for 2 h, followed by storing at 4° C. prior for further use.

### 1.4 Nanoparticle Characterization

To calculate mRNA encapsulation efficiency, Quant-iT RiboGreen RNA assay from invitrogen was used. Briefly, Ribogreen fluorescence for nanoparticles was compared in presence and absence of 10 mg/mL heparin and 2% triton in TE buffer for the polymeric nanoparticles. Whereas for LNP controls instead of heparin, 2% Triton in TE buffer was used. Fluorescence was quantified using a SpectraMax microplate reader (Molecular Device, USA) with 480 nm excitation wavelength and 520 nm emission wavelength. Nanoparticles were characterized for their size, PDI and charge using NanoBrook 90 Plus Zeta (Brookhaven, USA). For size measurement, particles were 200 times diluted in 1×PBS whereas for charge measurement particles were diluted 400 times in 0.1×PBS. To image nanoparticles for CryoTEM, nanoparticles were dialyzed against 0.1×PBS and 3 μL of the PNP solution was diluted with buffer and was placed onto a lacey copper grid coated with a continuous carbon film. And imaged with Talos Arctica electron microscope.

The ATP encapsulation efficiency of ATP (+) formulations were quantified by ATP determination kit (thermofisher).

### 1.4.1 TNS Assay

To calculate apparent pKₐ of formulations, the protocol previously described was utilized¹. Briefly, a series of buffers with pH values varying by 0.5 between 4.0 and 12 were prepared by titrating a solution containing 10 mM citrate, 10 mM phosphate, 10 mM borate, and 150 mM NaCl with 1.0 M HCl and aliquoted into a black 96-well plate. Formulations and 6-(p-toluidino)-2-naphthalenesulfonic acid were diluted into these buffers at final concentrations of 25 and 5.45 μM, respectively. The plate was covered and equilibrated at room temperature for 20 min. Fluorescence intensity was determined using a SpectraMax microplate reader (Molecular Device, USA) at 325 nm excitation/435 nm emission. The raw fluorescence values were normalized between 0 and 1 and fit using GraphPad Prism 10 to calculate pKₐ, which corresponds to the pH value at 50% max fluorescence.

### 1.5 In Vitro FLuc mRNA Expression

Cells were cultured in Dulbecco's Modified Eagles Medium (DMEM) supplemented with 10% fetal bovine serum (FBS) and 1% penicillin-streptomycin. Cells were seeded in 96-transparent well plates at a density of 10,000 cells per well for 24 h at 37° C. Then, the cell media was aspirated, 50 ng dose of FLuc mRNA encapsulated nanoparticles that had been formulated in different formulations were added into each well based on the total mRNA content in each formulation. After 24 h, 100 μL of Bright-Glo Luciferase Assay buffer (Promega) was added into each well The FLuc expression was quantified with a SpectraMax microplate reader (Molecular Device, USA). Expression levels were normalized to live cell signal. Cell viability was analyzed using Alamar Blue assay as per the protocol.

### 1.6 Cell Viability Assay

A549 cells were grown in DMEM supplemented with 10% fetal bovine serum (FBS) and 1% penicillin-streptomycin at 37° C. in a 5% CO₂ incubator. The cells were seeded in 96-transparent well plates at a density of 1×10⁴ cells per well in 100 μL media and incubated for 24 h. Subsequently, the cell media was removed and 100 μL of fresh cell media (for cell control) or 100 μL of 5C6 nanoparticles in different formulation conditions with 50 ng of mRNA were introduced into cells. Next day, the cell media was aspirated and 100 μL of aliquoted Alamar Blue reagent were added into each well followed by incubation for 3 h. Finally, the absorbance of the solution was measured using a SpectraMax microplate reader with 570 nm wavelength and a 600 nm reference wavelength. Untreated cell were the cells that only contains DMEM, used as background controls. The cell viability results were calculated by normalizing absorbance to the untreated cells.

### 1.7 Endosomal Escape Studies

A549 cells were seeded at a density of 4×10⁴ cells per well in μ-slide 8-well coverslip slides at 37° C. overnight to allow the cellular adhesion to the substrate. Next day, the cell media was aspirated, and 300 μL of Cy5 labelled FLuc mRNA encapsulating 5C6 C14 1K PEG, 5C6 C14 2K PEG, 5C6 C14 3K PEG, 5C6 C14 5K PEG and Moderna nanoparticles were introduced to the cells at a mRNA concentration of 500 ng/mL followed by incubation for 2 h at 37° C. Then, the cells were washed with DPBS three times, and 300 μL of 100 nM of LysoTracker Green DND-26 (that had been dissolved in prewarmed 37° C. culture media), was added. After 1 h, cells were washed with DPBS for three times and 1 μg/mL of Hoechst 33342 was introduced to cells for 15 min to stain the nucleus. Finally, the live cell imaging was performed by confocal laser scanning microscope (CLSM) (Leica SP8X, USA) with 60×water immersion objective. The images were further processed by WCIF Image J software. Experiments were performed in triplicate and five representative cell images (>30 cells) were used to calculate the PCC value.

### 1.8 Cell Association

A549 cells were seeded in a 24-transparent well plates at a density of 4×10⁴ cells per well and incubated in complete DMEM media at 37° C. overnight to allow the cellular adhesion to the substrate. On the next day, 200 μL 500 ng/mL of Cy5 labelled FLuc mRNA encapsulated 5C6 C14 1K PEG, 5C6 C14 2K PEG, 5C6 C14 3K PEG, 5C6 C14 5K PEG and Moderna nanoparticles were added to the cells followed by incubation for 2 h and 24 h at 37° C. Subsequently, the cells were washed with DPBS three times and dissociated using trypsin-EDTA. Finally, the samples are analyzed on Attune Flow Cytometer (Thermofisher Scientific, USA). The cell association was assessed by using the percentage of cells with stronger fluorescence intensity than the control untreated cells.

### 1.9 Endocytosis and Endosomal Escape Mechanism Studies

A549 cells were plated in a 24-transparent well plate at a density of 8×10⁴ cells per well for 24 h. Endocytosis inhibitors (pitstop 2, filipin from S. filipinensis, EIPA and cytochalasin D) were added to the cells to achieve final concentrations of 5, 12 μg/mL, 10 ug/mL, and 25 μM, respectively. For endosomal escape studies, bafilomycin A₁ with or without above mentioned endocytosis inhibitors was added to the cell to achieve final concentration of 200 nM. After 15 min incubation with the endocytosis inhibitors and/or proton sponge inhibitor, the 5C6 nanoparticles in different PEG molecular weight and Moderna lipid nanoparticles containing Cy5 labelled FLuc mRNA were added into each well followed by incubation for 2 h for endocytosis inhibitor studies and 24 h for endosomal escape studies. Cells treated with nanoparticles without adding inhibitors were used as control. The culture medium was then removed, and cells were washed three times with PBS and detached with trypsin-EDTA. The cells were analyzed using a Attune Flow Cytometer (Thermofisher Scientific, USA)

### 1.10 Calcein Release Studies

A549 cells were seeded at a density of 4×10⁴ cells per well in μ-slide 8-well coverslip slides at 37° C. overnight to allow the cellular adhesion to the substrate in normoxia environment. Then, the culture media was replaced with 180 μL of fresh media or media containing bafilomycin A₁ with or without Cytochalasin D. Calcein (20 μL, 1.5 mg/mL) was added to each well to obtain a final concentration of 150 μg mL-1 for calcein, 200 nM for bafilomycin A₁ and 25 uM Cytochalasin D. Nonfluorescently labeled 5C6 C14 1K PEG, 5C6 C14 2K PEG, 5C6 C14 3K PEG, 5C6 C14 5K PEG and Moderna nanoparticles were incubated with the treated cells for 4 h at a mRNA concentration of 1000 ng mL-1. After incubation, the samples were gently washed four times with DPBS to remove excess materials. Finally, cells were imaged and captured by CLSM.

### 1.11 In Vitro FLuc mRNA Expression in Normoxia and Hypoxia

A549 cells were seeded at a density of 1×10⁴ cells per well in 96-transparent well plates at 37° C. in either normoxia (21% O₂) or hypoxia (1% O₂) condition. After 24 h, the cell media was aspirated, and 100 μL of FLuc mRNA encapsulated 5C6 nanoparticles and Moderna lipid nanoparticles at 50 ng, 100 ng and 200 ng dose were added into each well, followed by incubation for 24 h under either normoxia or hypoxia condition. Finally, the in vitro FLuc mRNA expression was quantified by Bright-Glo Luciferase assay using a SpectraMax microplate reader with luminescence function.

### 1.12 Stability Studies

The stability of nanoparticles was determined by size changing of the nanoparticles in pH 7.4, pH 5 and DMEM with 10% FBS. Two different nanoparticle batches were formulated and dialyzed against 1×PBS; The size of formulated nanoparticles were measured. Then the formulated nanoparticles were further incubated in DMEM with 10% FBS, pH 7.4 and pH 5 solutions and stored at 4° C. and 37° C. for 72 h. Size was then measured again. Size change was normalized by dividing the size after 72 h incubation in various media and the size measured on the first day.

### 1.12 In Vivo Studies

All animal studies were approved by the UNC Institutional Animal Care and Use Committee, were consistent with local, state, and federal regulations as applicable, and were supported within the UNC Lineberger ASC at the University of North Carolina at Chapel Hill.

For intravenous dosing studies, FLuc mRNA encapsulated 5C6 nanoparticles were prepared to a final mRNA concentration of 0.1 mg mL⁻¹. PBS injection was used as a negative control. Each group was then injected via the tail vein of female C57 black 6 mice (Jackson Laboratory, 18-22 g) at a dose of 0.25 mg kg⁻¹. After 24 hours, mice were injected intraperitoneally with 130 μL of D-luciferin (30 mg mL⁻¹ in PBS). After 15 min, mice were euthanized, and organs (pancreas, spleen, liver, kidneys, ovaries, lung, and heart) were removed and imaged with an in-vivo imaging system (IVIS) (Perkin Elmer, Waltham, MA).

Luminescence was quantified using AuRA software (Spectral instruments imaging). The liver, spleen, and lung were then fixed in 10% neutral buffered formalin after imaging and were then routinely processed to paraffin. Histological sections were evaluated using hematoxylin and eosin stain. Briefly, paraffin embedded tissue blocks were sectioned at 5 μm onto positively charged slides. In order to proceed with histological staining, samples were first baked at 60 degrees Celsius for 60 minutes minimum and were then deparaffinized in xylene and hydrated with graded ethanol before continuing with the H&E stain. H&E stains were performed using the autostainer XL from Leica Biosystems. The sections were stained with Hematoxylin (Richard-Allen Scientific, 7211) for 2 mins and Eosin-Y (Richard-Allen Scientific, 7111) for 1 min. Clarifier 2 (7402) and Bluing (7111) solutions from Richard-Allen Scientific were used to differentiate the reaction. After staining, slides were then dehydrated, and cover slipped with Cytoseal 60 (8310-4, Thermo Fisher Scientific). Histological analysis was then performed on each sample.

### 1.13 Statistical Analysis

Statistical analyses were performed using GraphPad Prism (GraphPad Software, CA, USA). Unpaired and paired student t-test were used to compare differences between the two groups. p-value<0.05 were considered statistically significant and individual values (****p<0.001***p<0.001, **p<0.01, and *p<0.05) are indicated in figure legends.

The following reference is related to Experiments and Methods of Experiment 5: (1) Tilstra, G.; Couture-Senecal, J.; Lau, Y. M. A.; Manning, A. M.; Wong, D. S. M.; Janaeska, W. W.; Wuraola, T. A.; Pang, J.; Khan, O. F. Iterative Design of Ionizable Lipids for Intramuscular mRNA Delivery. J. Am. Chem. Soc. 2023, 145 (4), 2294-2304; doi: 10.1021/jacs.2c10670.

### Example 6. Additional Evaluation of LNPs

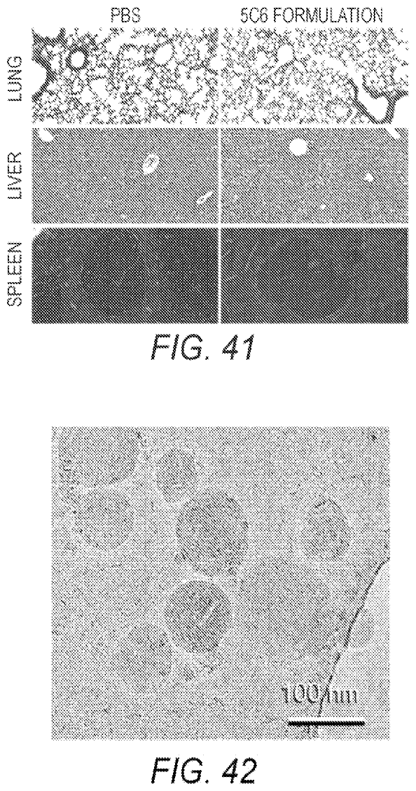

Blood panel results of liver and kidney function tests from 0.5 mg/kg dose of 5C6 injected intravenously demonstrate LNPs are well-tolerated (FIG. 40). Representative images of histology from representative sectioning shows tolerability in lung, liver and spleen tissues with PBS and 5C6 formulation (FIG. 41). Exemplary cryo-electron microscopy image of 5C6 indicating multilamellar structure of the particles with spherical morphology (FIG. 42).

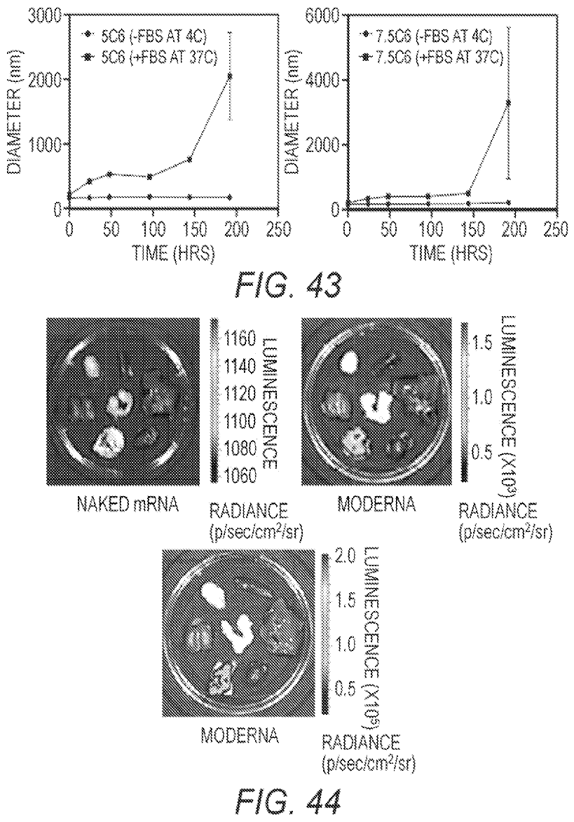

Results of stability studies for 5C6 (FIG. 43, left) and 7.5C6 (FIG. 43, right) complexed with mRNA with and without fetal bovine serum.

Imaging of mRNA concentration in mice organs resulting from Fluc mRNA intratracheally administered as naked mRNA, in Moderna LNP formulation, and 5C6 formulation taken 24 hours post dosing (FIG. 44).

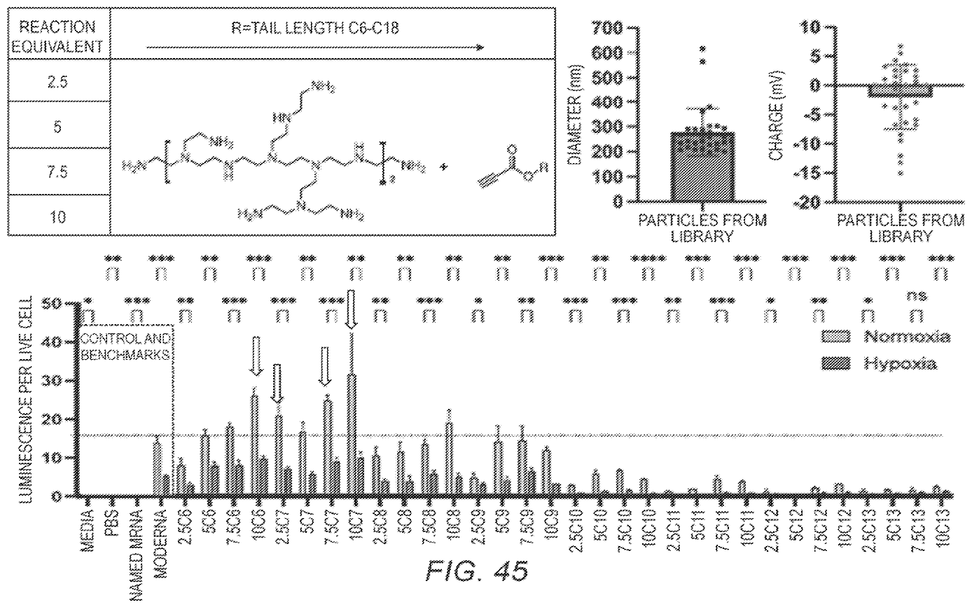

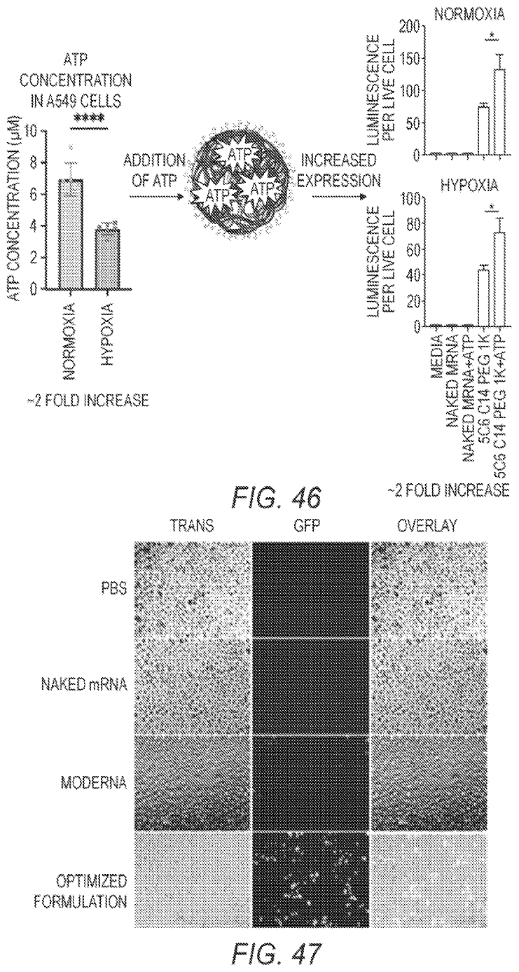

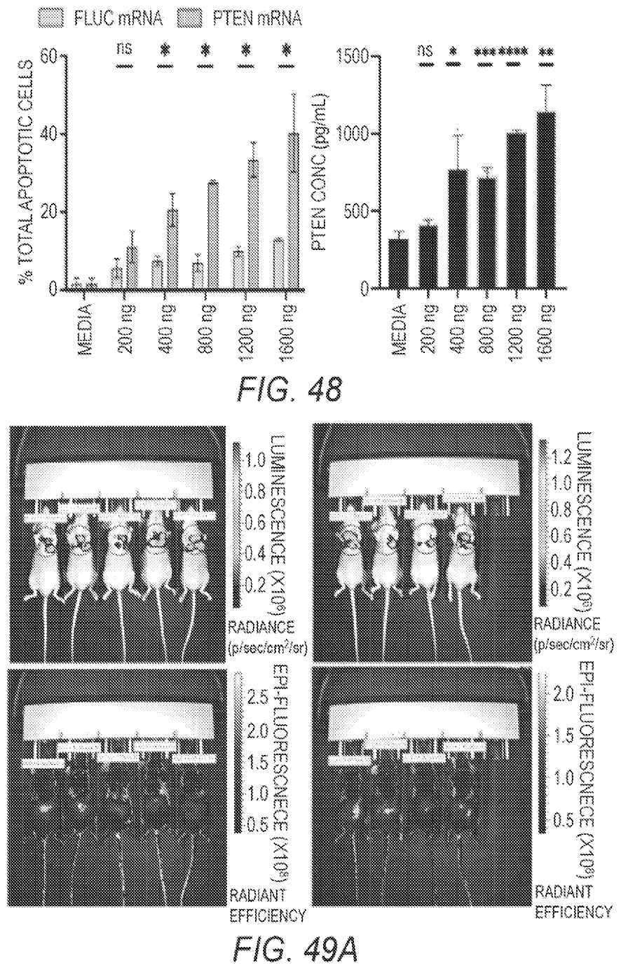

Measurement of the effect of hypoxia in A549 cell line with varying reaction equivalents of R tail length of C6-C13 in FLuc mRNA LNPs under normoxia and hypoxia conditions (FIG. 45). As shown in FIG. 46, LNPs provide tunability of protein expression with the addition of ATP to mRNA encapsulated LNP, resulting in approximately 2-fold increase in protein expression under hypoxic conditions Imaging demonstrates the versatility of 5C6 LNPs deliver multiple types of mRNA irrespective of coding sequence (FIG. 47). Delivery of PTEN mRNA in LNPs promoted apoptosis; as shown in FIG. 48, results of PTEN mRNA show dose response anti-cancer effect of PTEN mRNA delivery.

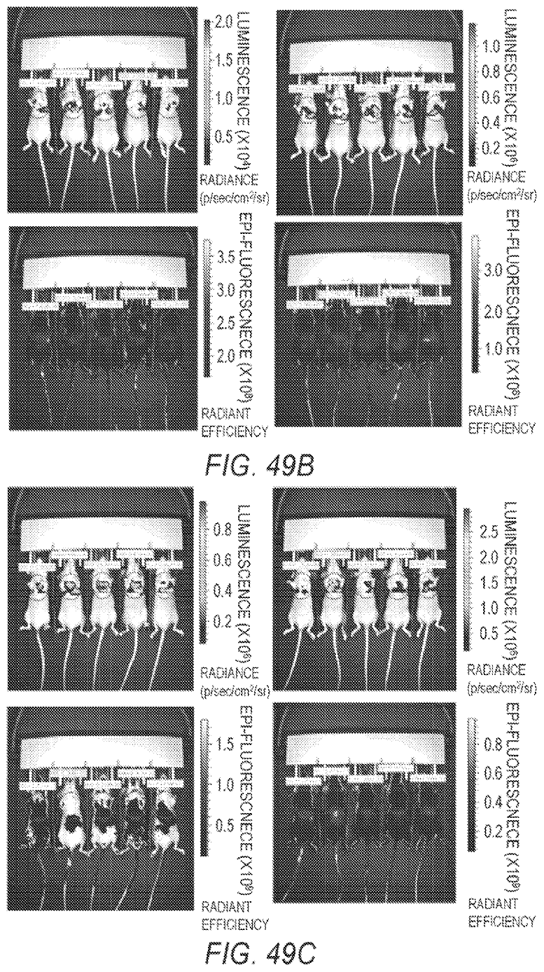

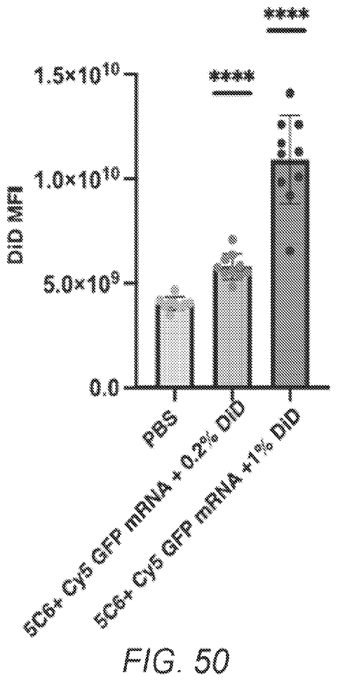

In lung cancer model of mice, intravenous administration of PBS (FIG. 49A), 0.2 mol % DiD (FIG. 49B); 1 mol % DiD (FIG. 49C) show tumor location in mice (upper row) and accumulation of LNPs (lower row). Measurement of the accumulation of 5C6 LNPs in lung tumor in an orthotopic mouse model with 0.25 mg/kg Cy5-GFP mRNA (FIG. 50).

The foregoing examples are illustrative of the present invention and are not to be construed as limiting thereof. Although the invention has been described in detail with reference to preferred embodiments, variations and modifications exist within the scope and spirit of the invention as described and defined in the following claims.

1. A polymer comprising a plurality of amine groups, wherein at least one amine group comprises a nitrogen atom directly bonded to a functional group according to Formula I: 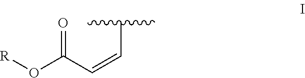
wherein R is C₆ to C₁₈ alkanyl, alkenyl or alkynyl.

2. A polymer formed by reacting a polyethyleneimine with an alkynoate according to Formula II: 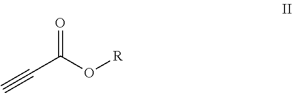
to thereby form a functionalized PEI polymer,
wherein R is C₆ to C₁₈ alkanyl, alkenyl or alkynyl.

3. The polymer of claim 2, wherein the alkynoate is provided at 2.5, 5, 7.5, or 10 molar equivalents to the polyethyleneimine.

4. The polymer of claim 3, wherein the alkynoate is a C₆₋₁₈ alkynoate.

5-58. (canceled)

59. The polymer of claim 2 wherein R is any enantiomer of C₆ to C₁₈ alkanyl, alkenyl or alkynyl.

60. The polymer of claim 2, wherein the polyethyleneimine is branched or unbranched, optionally
wherein the PEI is branched with a number average molecular weight (Mₙ) of about 400 to about 750,000 g/mol, or
wherein the PEI is branched with a number average molecular weight (Mₙ) of about 600 g/mol, or
wherein the PEI is linear with a number average molecular weight (Mₙ) of about 400 to about 30,000 g/mol.

61-190. (canceled)

191. A nanoparticle comprising the polymer of claim 2 and a cargo.

192. The nanoparticle of claim 191, further comprising a polyethylene glycol (PEG) or derivative thereof.

193. The nanoparticle of claim 192, wherein the PEG is provided at about 0.25% by weight of the polymer.

194. The nanoparticle of claim 191, wherein the cargo is a biologically active agent, imaging agent, or therapeutic agent,
optionally wherein the cargo is a nucleic acid, a protein, a complex of a nucleic acid and a protein, a carbohydrate, a lipid, or a small molecule, optionally wherein the nucleic acid is an RNA (e.g., an mRNA, antisense oligonucleotide, or siRNA), or
a gene modulating agent, optionally a CRISPR-Cas system, a zinc finger nuclease, a TALEN, or a meganuclease.

195-269. (canceled)

270. A composition comprising the polymer of claim 2, and optionally an excipient, optionally wherein the excipient comprises a nucleoside triphosphate molecule, derivative, analog, metabolite, or precursor thereof.

271. (canceled)

272. A composition comprising the nanoparticle of claim 191, and optionally an excipient, optionally wherein the excipient comprises a nucleoside triphosphate molecule, derivative, analog, metabolite, or precursor thereof.

273. (canceled)

274. A method of delivering a cargo to a target cell, comprising contacting the target cell with the nanoparticle of claim 191.

275. A method of modulating expression of a gene in a target cell, comprising contacting the target cell with the nanoparticle of claim 191.

276-278. (canceled)

279. The method of claim 274, wherein the target cell is a liver cell, kidney cell, heart cell, reproductive organ cell, lung cell, or a spleen cell.

280. A method of delivering a cargo to a subject in a tissue specific manner, comprising contacting the tissue with the nanoparticle of claim 192, optionally wherein the tissue is lung, heart, kidney, reproductive organ, liver, or spleen.

281. (canceled)

282. A method of treating a disease or disorder in a subject in need thereof, comprising administering to the subject the nanoparticle of claim 192, wherein the cargo provides a therapeutic effect for the disease or disorder.

283. A method of vaccinating a subject in need thereof, comprising administering to the subject the nanoparticle of claim 192, wherein the cargo provides an immunostimulatory effect.

284. A method of making the polymer of claim 2, comprising adding a solution of alkynoate in a solvent to PEI to make a mixture, stirring the mixture at room temperature thereby forming the polymer, compound, or composition in the solvent, and evaporating the solvent, thereby producing the polymer, compound, or composition.

285-295. (canceled)

296. A method of making a nanoparticle, comprising adding a cargo to the polymer of claim 2, wherein the nanoparticle self-assembles via electrostatic interactions between the cargo and the polymer.

297-305. (canceled)
